SEQUENCE LISTING (467 sequences)

SEQ ID NO: 1 - DNA, 328 bp, synthetic construct
  source 1..328: mol_type = other DNA
tctttccgtctgacggtgtacgtcgcggagtaaacctacacagacctcagaaccttcgaactgatgggatgcaagaggatgtttacctggaactctcgaacaaacctccaagatcgtcccctcgcgtcgatgagcatatgggaactggcttctggccaggaggagatagcccctaccagcaggagaagctggctcgcgcgtcgaagccctccctgcgtgtacctcgccactccctccttcccctgtgggcggatcggtcggtctagtcggcttagttgggaccgctagttaccccactgtctacagcgtcggtctagcgggagtgtag

SEQ ID NO: 2 - DNA, 299 bp, synthetic construct
  source 1..299: mol_type = other DNA
tctctgccccagagcgatacaacgggtccgacctcacgtcaccgataagtgtccgcgctagggtgatgactagtcgtgccctcaaaactggacgaggcaaaggctggacccggccaagtggggaggaatccgttggaccaccagggggcgagggccctccagtggtataactacggcttgaatcacgcctgtgggctagccgtatcgcgtgatgtcgggtcttgaggacctgagttcgctaggaggtcggagtcggagggctcatcgaccctgatgtccgtgcgcggtggcgcgggccg

SEQ ID NO: 3 - DNA, 300 bp, synthetic construct
  source 1..300: mol_type = other DNA
ttctctgccccagagcgatacaacgagtccgacctcacgtcaccgataagtgtccgcgctagggtgatgactagtcgtgccctcaaaactggacgaggcaaaggctggacccggccaagtggggaggaatccgttggaccaccagggggcgagggccctccagtggtataactacggcttgaatcacgcctgtgggctagccgtatcgcgtgatgtcgggtcttgaggacccgagttcgctaggagggtggagtcggagggctcatcgaccctgatgtccgtgcgcggtggcgcgggccg

SEQ ID NO: 4 - DNA, 321 bp, synthetic construct
  source 1..321: mol_type = other DNA
ttacctataaacagagacattactcgagtccgctttccttgacaccgatattcttttaatcccctgtggtcatcgtaatgtctgaagatgactagtcgtgccctcaaaactggacgaggcaaaggctggacccggccaagtggggaggaatccgttggaccaccagggggcgagggccctccagtggtataactacggcttgaatcacgcctgtgggctagccgtatcgcgtgatgtcgggtcttgaggacctgagttcgctaggaggtcggagtcggagggctcatcgaccctggtgtccgtgcgcggtggcgcgggccg

SEQ ID NO: 5 - DNA, 333 bp, synthetic construct
  source 1..333: mol_type = other DNA
ccgcctcctctcatcagacttaacccaatactccaggggacgccccatggagtggagtcggtaacttgagtgaagcgaccggcactcagacaaggttcgaggccgtttcctccgtaggcggcccggggaggggcttcccgccccaggtgccgtagaggacgggtcagactggagcgcgcctcggggcaagagacccttgagtggaggggcttcgagtccctctcgggacaatcccggcggagaccgggatcagagtctggaagggttccctgtaccctcacctcactgtcctgcgtgagtcgagcaccggggtgactactcgaagggaggcgg

SEQ ID NO: 6 - DNA, 699 bp, synthetic construct
  source 1..699: mol_type = other DNA
gtacgactgtggggcaaagacggtttttttttttttttttttttttttttcggacctcaccgcacacgcggaaattcggggccgatgagccctcccgaatccgccctctgagcgaactcgggccctccgtctacaacgatcctcggctctagcgcggtgacgttaggtcggccccggccgtctgtcttgttctctgggcctctggggcagacccccaccctcaccgcccgccctttgccgctgttttctccctcgctcttaccctcttttgcgtttttctctgttatcctccacggtgtgtcgtagggggcggagcgcggttctgattttaatttttcggtggggctcttttgggttctcggcagtggcgactttgtcctccgcttttggaaagagcgctgtacggttcgtcctcgcttgtcgggtctttgcggggcctcaaatgctcgatcagagacagtaactcggtagttcttcctttttggtgagtctggcgcaagagagggagagtgaggggttatgcctctcttcttgctagtagttaccgactgccgtcaacgtcggttcgttgcggtctttcggccgaagtgcgagtcctcttttgcgatggagagaaggagcaccaaaagccacgagatgtgcaagtctctttgaagagatcattgtgtgatatctttactagggactttcatatcagaa

SEQ ID NO: 7 - DNA, 328 bp, synthetic construct
  source 1..328: mol_type = other DNA
ctacactcccgctagaccgacgctgtagacagtggggtaactagcggtcccaactaagccgactagaccgaccgatccgcccacaggggaaggagggagtggcgaggtacacgcagggagggcttcgacgcgcgagccagcttctcctgctggtaggggctatctcctcctggccagaagccagttcccatatgctcatcgacgcgaggggacgatcttggaggtttgttcgagagttccaggtaaacatcctcttgcatcccatcagttcgaaggttctgaggtctgtgtaggtttactccgcgacgtacaccgtcagacggaaaga

SEQ ID NO: 8 - DNA, 50 bp, synthetic construct
  source 1..50: mol_type = other DNA
ctgaggtctgtgtaggtttactccgcgacgtacaccgtcagacggaaaga

SEQ ID NO: 9 - DNA, 50 bp, synthetic construct
  source 1..50: mol_type = other DNA
gatcttggaggtttgttcgagagttccaggtaaacatcctcttgcatccc

SEQ ID NO: 10 - DNA, 50 bp, synthetic construct
  source 1..50: mol_type = other DNA
gctggtaggggctatctcctcctggccagaagccagttcccatatgctca

SEQ ID NO: 11 - DNA, 50 bp, synthetic construct
  source 1..50: mol_type = other DNA
caggggaaggagggagtggcgaggtacacgcagggagggcttcgacgcgc

SEQ ID NO: 12 - DNA, 50 bp, synthetic construct
  source 1..50: mol_type = other DNA
gacgctgtagacagtggggtaactagcggtcccaactaagccgactagac

SEQ ID NO: 13 - DNA, 50 bp, synthetic construct
  source 1..50: mol_type = other DNA
acttatcggtgacgtgaggtcggacccgttgtatcgctctggggcagaga

SEQ ID NO: 14 - DNA, 50 bp, synthetic construct
  source 1..50: mol_type = other DNA
ggccgggtccagcctttgcctcgtccagttttgagggcacgactagtcat

SEQ ID NO: 15 - DNA, 50 bp, synthetic construct
  source 1..50: mol_type = other DNA
caagccgtagttataccactggagggccctcgccccctggtggtccaacg

SEQ ID NO: 16 - DNA, 50 bp, synthetic construct
  source 1..50: mol_type = other DNA
ctcctagcgaactcaggtcctcaagacccgacatcacgcgatacggctag

SEQ ID NO: 17 - DNA, 50 bp, synthetic construct
  source 1..50: mol_type = other DNA
cttatcggtgacgtgaggtcggactcgttgtatcgctctggggcagagaa

SEQ ID NO: 18 - DNA, 50 bp, synthetic construct
  source 1..50: mol_type = other DNA
gccgggtccagcctttgcctcgtccagttttgagggcacgactagtcatc

SEQ ID NO: 19 - DNA, 50 bp, synthetic construct
  source 1..50: mol_type = other DNA
aagccgtagttataccactggagggccctcgccccctggtggtccaacgg

SEQ ID NO: 20 - DNA, 50 bp, synthetic construct
  source 1..50: mol_type = other DNA
tcctagcgaactcgggtcctcaagacccgacatcacgcgatacggctagc

SEQ ID NO: 21 - DNA, 50 bp, synthetic construct
  source 1..50: mol_type = other DNA
tatcggtgtcaaggaaagcggactcgagtaatgtctctgtttataggtaa

SEQ ID NO: 22 - DNA, 50 bp, synthetic construct
  source 1..50: mol_type = other DNA
gtccagttttgagggcacgactagtcatcttcagacattacgatgaccac

SEQ ID NO: 23 - DNA, 50 bp, synthetic construct
  source 1..50: mol_type = other DNA
agggccctcgccccctggtggtccaacggattcctccccacttggccggg

SEQ ID NO: 24 - DNA, 50 bp, synthetic construct
  source 1..50: mol_type = other DNA
aagacccgacatcacgcgatacggctagcccacaggcgtgattcaagccg

SEQ ID NO: 25 - DNA, 50 bp, synthetic construct
  source 1..50: mol_type = other DNA
accgcgcacggacaccagggtcgatgagccctccgactccgacctcctag

SEQ ID NO: 26 - DNA, 50 bp, synthetic construct
  source 1..50: mol_type = other DNA
ccatggggcgtcccctggagtattgggttaagtctgatgagaggaggcgg

SEQ ID NO: 27 - DNA, 50 bp, synthetic construct
  source 1..50: mol_type = other DNA
acggaggaaacggcctcgaaccttgtctgagtgccggtcgcttcactcaa

SEQ ID NO: 28 - DNA, 50 bp, synthetic construct
  source 1..50: mol_type = other DNA
ggcgcgctccagtctgacccgtcctctacggcacctggggcgggaagccc

SEQ ID NO: 29 - DNA, 50 bp, synthetic construct
  source 1..50: mol_type = other DNA
ggtctccgccgggattgtcccgagagggactcgaagcccctccactcaag

SEQ ID NO: 30 - DNA, 50 bp, synthetic construct
  source 1..50: mol_type = other DNA
ccggtgctcgactcacgcaggacagtgaggtgagggtacagggaaccctt

SEQ ID NO: 31 - DNA, 50 bp, synthetic construct
  source 1..50: mol_type = other DNA
aacagcggcaaagggcgggcggtgagggtgggggtctgccccagaggccc

SEQ ID NO: 32 - DNA, 50 bp, synthetic construct
  source 1..50: mol_type = other DNA
ccccctacgacacaccgtggaggataacagagaaaaacgcaaaagagggt

SEQ ID NO: 33 - DNA, 50 bp, synthetic construct
  source 1..50: mol_type = other DNA
aaagtcgccactgccgagaacccaaaagagccccaccgaaaaattaaaat

SEQ ID NO: 34 - DNA, 50 bp, synthetic construct
  source 1..50: mol_type = other DNA
gaggccccgcaaagacccgacaagcgaggacgaaccgtacagcgctcttt

SEQ ID NO: 35 - DNA, 50 bp, synthetic construct
  source 1..50: mol_type = other DNA
cctctcttgcgccagactcaccaaaaaggaagaactaccgagttactgtc

SEQ ID NO: 36 - DNA, 50 bp, synthetic construct
  source 1..50: mol_type = other DNA
aacgaaccgacgttgacggcagtcggtaactactagcaagaagagaggca

SEQ ID NO: 37 - DNA, 50 bp, synthetic construct
  source 1..50: mol_type = other DNA
tcgtggcttttggtgctccttctctccatcgcaaaagaggactcgcactt

SEQ ID NO: 38 - DNA, 50 bp, synthetic construct
  source 1..50: mol_type = other DNA
ttctgatatgaaagtccctagtaaagatatcacacaatgatctcttcaaa

SEQ ID NO: 39 - DNA, 50 bp, synthetic construct
  source 1..50: mol_type = other DNA
cggcccgcgccaccgcgcacggacatcagggtcgatgagccctccgactc

SEQ ID NO: 40 - DNA, 50 bp, synthetic construct
  source 1..50: mol_type = other DNA
ctctgttcagcattgtaccattcacatgacctttcacgtgaacctgcttg

SEQ ID NO: 41 - DNA, 50 bp, synthetic construct
  source 1..50: mol_type = other DNA
ggcagtgggaggagttcatatgaagtttcctgtaaattgattttggggat

SEQ ID NO: 42 - DNA, 50 bp, synthetic construct
  source 1..50: mol_type = other DNA
ctaaatcgtcatttgactctcatctcacgaatcaacttgtcccgggactt

SEQ ID NO: 43 - DNA, 50 bp, synthetic construct
  source 1..50: mol_type = other DNA
ttacccgatgtaaaagatggggtcttttgatgctatcgggaatactttga

SEQ ID NO: 44 - DNA, 50 bp, synthetic construct
  source 1..50: mol_type = other DNA
tgtttcattcgcgttcatgggtgcatttctgcaatccagttccacatcgg

SEQ ID NO: 45 - DNA, 50 bp, synthetic construct
  source 1..50: mol_type = other DNA
ctagttggagtggtggagaacgagtcggatatatggcggtagaagtcgtt

SEQ ID NO: 46 - DNA, 50 bp, synthetic construct
  source 1..50: mol_type = other DNA
tgagtttcctggaccgccacgaagtatagggagatctcctcggacaagac

SEQ ID NO: 47 - DNA, 50 bp, synthetic construct
  source 1..50: mol_type = other DNA
cgggatttggagttgtcaatttagttgttttgacgagcggtcttgtgatg

SEQ ID NO: 48 - DNA, 50 bp, synthetic construct
  source 1..50: mol_type = other DNA
gaaattgtatagacttgtgtgttatcgattctgggtttgaccctaatcta

SEQ ID NO: 49 - DNA, 50 bp, synthetic construct
  source 1..50: mol_type = other DNA
tatttcgattttgagtggactcaacattttttgaggtcaactgtgtttta

SEQ ID NO: 50 - DNA, 50 bp, synthetic construct
  source 1..50: mol_type = other DNA
cagtgtgctaattgggttcagttatcttcggccgcatttctcacaaaatc

SEQ ID NO: 51 - DNA, 50 bp, synthetic construct
  source 1..50: mol_type = other DNA
tatttgctttcaaattgattcgatatgattggggtcccaaccagttaaag

SEQ ID NO: 52 - DNA, 50 bp, synthetic construct
  source 1..50: mol_type = other DNA
tacgtcgagttttgcgaatcggatcggtgtgggggtgccctttgtcgtca

SEQ ID NO: 53 - DNA, 50 bp, synthetic construct
  source 1..50: mol_type = other DNA
ggcaaggtcactcaagtgggagatttagtggtgctagttttccctgttcg

SEQ ID NO: 54 - DNA, 50 bp, synthetic construct
  source 1..50: mol_type = other DNA
atttgtttatccaaaccaggatcggaaagataatcgagaatcattctaat

SEQ ID NO: 55 - DNA, 50 bp, synthetic construct
  source 1..50: mol_type = other DNA
tatagtagagttgaatcataatatgggtgtgggtgggttcttgtcccaaa

SEQ ID NO: 56 - DNA, 50 bp, synthetic construct
  source 1..50: mol_type = other DNA
aggagggacatgctttcctgttctctttattccggatgaagtgtttcgcg

SEQ ID NO: 57 - DNA, 50 bp, synthetic construct
  source 1..50: mol_type = other DNA
aatttcaggatgcactagactcaagtctggcctcattaggtccagccaaa

SEQ ID NO: 58 - DNA, 50 bp, synthetic construct
  source 1..50: mol_type = other DNA
tggagctacaacctagtcctgtagggctaccacgtcggcgataatttcca

SEQ ID NO: 59 - DNA, 50 bp, synthetic construct
  source 1..50: mol_type = other DNA
tgttcaatgggatccctattgtcgcgttaggataagatctcaggtatagt

SEQ ID NO: 60 - DNA, 50 bp, synthetic construct
  source 1..50: mol_type = other DNA
acgattctgaagtggtcagtttcgcttgatgatatgagttaactaggtta

SEQ ID NO: 61 - DNA, 50 bp, synthetic construct
  source 1..50: mol_type = other DNA
gtttggacgtaatttttaaagccaaccccgctggagcctcgtcttgggtt

SEQ ID NO: 62 - DNA, 50 bp, synthetic construct
  source 1..50: mol_type = other DNA
ctgggatacctcgaaattaaataattacgtttgtcatggattgtttgggt

SEQ ID NO: 63 - DNA, 50 bp, synthetic construct
  source 1..50: mol_type = other DNA
gaatgaaaattggtcactttaactggacgggcacttctccgcccgtactg

SEQ ID NO: 64 - DNA, 50 bp, synthetic construct
  source 1..50: mol_type = other DNA
atcgtattagtgaacaaggaatttatccctggacatacttaccgaggtgc

SEQ ID NO: 65 - DNA, 50 bp, synthetic construct
  source 1..50: mol_type = other DNA
ataatctccgtggcggacgggtcactgtgtacaaattgccggcgccatgg

SEQ ID NO: 66 - DNA, 50 bp, synthetic construct
  source 1..50: mol_type = other DNA
cattttccttgagccgtttggaatggggcggacaaatggtttttgtagtg

SEQ ID NO: 67 - DNA, 50 bp, synthetic construct
  source 1..50: mol_type = other DNA
tgttcagtaataatgggagtgacagttgggttgtgtccgtacgagtattc

SEQ ID NO: 68 - DNA, 50 bp, synthetic construct
  source 1..50: mol_type = other DNA
gtattcggacgcagtctagttttgtgacttgactgttaattgtcgggtta

SEQ ID NO: 69 - DNA, 50 bp, synthetic construct
  source 1..50: mol_type = other DNA
ctggttagatagtgggatatcttcttgattacaatcatattcattgtact

SEQ ID NO: 70 - DNA, 50 bp, synthetic construct
  source 1..50: mol_type = other DNA
aagttcgagttgtgggtgatggattttttagggtttgtatattgacttga

SEQ ID NO: 71 - DNA, 50 bp, synthetic construct
  source 1..50: mol_type = other DNA
acatctctctcattttttaaattgtgggtatcatccggattttcgtcggt

SEQ ID NO: 72 - DNA, 50 bp, synthetic construct
  source 1..50: mol_type = other DNA
taggggaacatttaaattgacaatcaggtttctccttgtcgagaaacctg

SEQ ID NO: 73 - DNA, 50 bp, synthetic construct
  source 1..50: mol_type = other DNA
cactatcgaccaacaggttctatcttagaatcaagttgaaatttaaacgg

SEQ ID NO: 74 - DNA, 50 bp, synthetic construct
  source 1..50: mol_type = other DNA
gtgggcagatacatcgttttatcacccttctaaatatccatctccgctgt

SEQ ID NO: 75 - DNA, 50 bp, synthetic construct
  source 1..50: mol_type = other DNA
cgttcctctcggtttcgattctgggggctttggtctgctcgatggattct

SEQ ID NO: 76 - DNA, 50 bp, synthetic construct
  source 1..50: mol_type = other DNA
ttcgtattatatcgttcctgattggggatatggaagacgtattacttaat

SEQ ID NO: 77 - DNA, 50 bp, synthetic construct
  source 1..50: mol_type = other DNA
gctatctttaactttggaccgcgttatctatatcatggcgttccctttct

SEQ ID NO: 78 - DNA, 50 bp, synthetic construct
  source 1..50: mol_type = other DNA
ggtttgggtgaggtggaatgatggtctgttggaatcggtttggtaaatgg

SEQ ID NO: 79 - DNA, 50 bp, synthetic construct
  source 1..50: mol_type = other DNA
cattttcagcattgttccaaaggcatccacttggacgccttcctagtaat

SEQ ID NO: 80 - DNA, 50 bp, synthetic construct
  source 1..50: mol_type = other DNA
ggccccagccgggtgccgggaccgcctcgcgactcttctgccagcttgaa

SEQ ID NO: 81 - DNA, 50 bp, synthetic construct
  source 1..50: mol_type = other DNA
aaacatgtgtggcgggcagcgatgatggctaacctaccaaatcactccgg

SEQ ID NO: 82 - DNA, 50 bp, synthetic construct
  source 1..50: mol_type = other DNA
gggtacttgctcccttaagggctcattcacgcccagtattcgaacgcaac

SEQ ID NO: 83 - DNA, 50 bp, synthetic construct
  source 1..50: mol_type = other DNA
gcggccgtccgcgcccattgggcaacttggggtaagcactacccctagcc

SEQ ID NO: 84 - DNA, 50 bp, synthetic construct
  source 1..50: mol_type = other DNA
ctacgggaatctacaggccccgacgtgcgcgcgatgtgactgaccgagtc

SEQ ID NO: 85 - DNA, 50 bp, synthetic construct
  source 1..50: mol_type = other DNA
agggggttgaagaatctccctgttcaccgcaagtcggtgggctctaactc

SEQ ID NO: 86 - DNA, 50 bp, synthetic construct
  source 1..50: mol_type = other DNA
aattaaggctattgcttgctctgagaccgtacgattgatcaatgcgctgg

SEQ ID NO: 87 - DNA, 50 bp, synthetic construct
  source 1..50: mol_type = other DNA
cgagaaagagctaaggcacccaccaccacgtaccggcaagaatcaaccac

SEQ ID NO: 88 - DNA, 50 bp, synthetic construct
  source 1..50: mol_type = other DNA
gaattaaactgagttgtgccctttggagtgggccgggcctgtgcctgtcc

SEQ ID NO: 89 - DNA, 50 bp, synthetic construct
  source 1..50: mol_type = other DNA
ataccaacgtttcgactttgaatttccttaactgccttcccgtggtggtc

SEQ ID NO: 90 - DNA, 50 bp, synthetic construct
  source 1..50: mol_type = other DNA
ccgcaataagggtactgggcggcccgtcgaaggccctttggtttcagaaa

SEQ ID NO: 91 - DNA, 50 bp, synthetic construct
  source 1..50: mol_type = other DNA
tccaagcttctgctagtctatggcagcatcaaggctggtatttgctacgg

SEQ ID NO: 92 - DNA, 50 bp, synthetic construct
  source 1..50: mol_type = other DNA
gcgttctgcctggtctcgctttcgtaaacggttcttacaaaagtaattag

SEQ ID NO: 93 - DNA, 50 bp, synthetic construct
  source 1..50: mol_type = other DNA
gtactaattctccctgccggcccccgtaagcataacgcggcgatctccac

SEQ ID NO: 94 - DNA, 50 bp, synthetic construct
  source 1..50: mol_type = other DNA
tatggcgtcgatccttattaccttatcctggcgccaagataaaacaacca

SEQ ID NO: 95 - DNA, 50 bp, synthetic construct
  source 1..50: mol_type = other DNA
ggcgccccgggcttcgcaaatgaaacttttttaatctcacaagtttcgtc

SEQ ID NO: 96 - DNA, 50 bp, synthetic construct
  source 1..50: mol_type = other DNA
cgctcggtggcgggcaggggcggggaacggagagccgcgggggagctacg

SEQ ID NO: 97 - DNA, 50 bp, synthetic construct
  source 1..50: mol_type = other DNA
atttcaacgacgtcaatttttcgagcatcaacctagaaccctcgcccgcc

SEQ ID NO: 98 - DNA, 50 bp, synthetic construct
  source 1..50: mol_type = other DNA
tcctaggtaacctcccgttcagaccacggtcgtcggcgccattaaggtcg

SEQ ID NO: 99 - DNA, 50 bp, synthetic construct
  source 1..50: mol_type = other DNA
ttattgttatgtcctgagaaagctccgggacattaaccttactcaggtga

SEQ ID NO: 100 - DNA, 50 bp, synthetic construct
  source 1..50: mol_type = other DNA
gtgtaggttccttccgtcgtccgcgcgtttaatgggtgagggctgggccc

SEQ ID NO: 101 - DNA, 50 bp, synthetic construct
  source 1..50: mol_type = other DNA
taccactggtgcccactgccccttagtcccaagctaaggcctctccctcg

SEQ ID NO: 102 - DNA, 50 bp, synthetic construct
  source 1..50: mol_type = other DNA
caccgccgctgctgggtaagcttgcagacgggatagttgaaagctaccat

SEQ ID NO: 103 - DNA, 50 bp, synthetic construct
  source 1..50: mol_type = other DNA
cggccccccgcccgcggccgccgaaaccactgagatctattggagcccgg

SEQ ID NO: 104 - DNA, 50 bp, synthetic construct
  source 1..50: mol_type = other DNA
aagcgccccccctacgcacgtaaatagtctagttttggttgggccagtcg

SEQ ID NO: 105 - DNA, 50 bp, synthetic construct
  source 1..50: mol_type = other DNA
aggagaggatgaacctattgacaccattaagatctcgattatgtacggct

SEQ ID NO: 106 - DNA, 50 bp, synthetic construct
  source 1..50: mol_type = other DNA
gtgccggccatgtcactttgacgcttaccgagtaatttagtcaataccaa

SEQ ID NO: 107 - DNA, 50 bp, synthetic construct
  source 1..50: mol_type = other DNA
tggaccaactaggacggtcatcgtatacgaacagagtttctaattcggta

SEQ ID NO: 108 - DNA, 50 bp, synthetic construct
  source 1..50: mol_type = other DNA
gagggagcgacgctagataactttcagtcgggagctgtgttcccaaacag

SEQ ID NO: 109 - DNA, 50 bp, synthetic construct
  source 1..50: mol_type = other DNA
tggaccgcgatttggtaagcatctgctggacgaagacccagccccaaagc

SEQ ID NO: 110 - DNA, 50 bp, synthetic construct
  source 1..50: mol_type = other DNA
ctttgccccgcgccggcctttccgccggcgggggagcgggcagtgcgtgg

SEQ ID NO: 111 - DNA, 50 bp, synthetic construct
  source 1..50: mol_type = other DNA
gccctcccgcgcacggggcggcgcgcggccctggccccaggccacgcctc

SEQ ID NO: 112 - DNA, 50 bp, synthetic construct
  source 1..50: mol_type = other DNA
gagcctatcggccagggggcggacaggggcggccgcccggcgggggggga

SEQ ID NO: 113 - DNA, 50 bp, synthetic construct
  source 1..50: mol_type = other DNA
ttgcggagattcagtcttagggcgggtccgcttgctatgccgtcgcggcg

SEQ ID NO: 114 - DNA, 50 bp, synthetic construct
  source 1..50: mol_type = other DNA
taaaccacatacacgaaccgactcctcggttaccccgcttcgatggtaga

SEQ ID NO: 115 - DNA, 50 bp, synthetic construct
  source 1..50: mol_type = other DNA
ggatgactactacacaacaacggtaccattaggacgagtcatgctctcct

SEQ ID NO: 116 - DNA, 50 bp, synthetic construct
  source 1..50: mol_type = other DNA
taacaagtgggtgattatcccttgcactcgacccaaatctggcagcactc

SEQ ID NO: 117 - DNA, 50 bp, synthetic construct
  source 1..50: mol_type = other DNA
agcgaaaaactaggaagctacagccgagaaggatagtaacacttcgtctt

SEQ ID NO: 118 - DNA, 50 bp, synthetic construct
  source 1..50: mol_type = other DNA
tcctccacagtcttttcaatggtgtccctattgaccgaacaccgccggtt

SEQ ID NO: 119 - DNA, 50 bp, synthetic construct
  source 1..50: mol_type = other DNA
aagtcatgcttatgtctggcactttcgccccggagtgctaggaagactgg

SEQ ID NO: 120 - DNA, 50 bp, synthetic construct
  source 1..50: mol_type = other DNA
cgctcgagtccctcctgtctttggagggcacctcgtcttcccgttttcga

SEQ ID NO: 121 - DNA, 50 bp, synthetic construct
  source 1..50: mol_type = other DNA
gtcacggtccacccctcaaactgaccccgccatgtggacagtttgccatt

SEQ ID NO: 122 - DNA, 50 bp, synthetic construct
  source 1..50: mol_type = other DNA
tcgggctccccgagagcgaagaccgcggttcgcgggcgggccggcccgcg

SEQ ID NO: 123 - DNA, 50 bp, synthetic construct
  source 1..50: mol_type = other DNA
gcccggcggccactttatggtgatgagactagcaaaaaagtgactgggcc

SEQ ID NO: 124 - DNA, 50 bp, synthetic construct
  source 1..50: mol_type = other DNA
caccctccgggggccgcgggggggccacaggggcgctccccgggccccgc

SEQ ID NO: 125 - DNA, 50 bp, synthetic construct
  source 1..50: mol_type = other DNA
ttcttctgggacaactcgaactgagatcagaccgtgccacttctctgtac

SEQ ID NO: 126 - DNA, 50 bp, synthetic construct
  source 1..50: mol_type = other DNA
gacagggatggatgataggtcgctttggtgtcggttcccttgcccgaacc

SEQ ID NO: 127 - DNA, 50 bp, synthetic construct
  source 1..50: mol_type = other DNA
aattccatcggtttacggagcagtagattaatcactgcgcgtacttacct

SEQ ID NO: 128 - DNA, 50 bp, synthetic construct
  source 1..50: mol_type = other DNA
agacttacagtttcacttctttaagttacttcgcgcccatttgccgccct

SEQ ID NO: 129 - DNA, 50 bp, synthetic construct
  source 1..50: mol_type = other DNA
ttaattttgtttcgtagcgcttccgggcgccgcccacaactgcgctacac

SEQ ID NO: 130 - DNA, 50 bp, synthetic construct
  source 1..50: mol_type = other DNA
gagcggagccggccgcggatcgtcggctgaatcttgaccacgcctggtcc

SEQ ID NO: 131 - DNA, 50 bp, synthetic construct
  source 1..50: mol_type = other DNA
cgcctccccggcgcccggccagggggggcggcccaggcgggggccccggc

SEQ ID NO: 132 - DNA, 50 bp, synthetic construct
  source 1..50: mol_type = other DNA
ggggccggggcaggcggggggcaaggggggaggaggagccgcgcgccgcc

SEQ ID NO: 133 - DNA, 50 bp, synthetic construct
  source 1..50: mol_type = other DNA
cctcgccagcccgccgccgccagccgcccgccgccccgccccgccaagca

SEQ ID NO: 134 - DNA, 50 bp, synthetic construct
  source 1..50: mol_type = other DNA
gccgccgcgccgcgcggggggtgggggtggggtgcagagccagcgcgcgc

SEQ ID NO: 135 - DNA, 50 bp, synthetic construct
  source 1..50: mol_type = other DNA
cggcccgggaagggcacctagcggggtcgacgccgcccgcagcgccggcg

SEQ ID NO: 136 - DNA, 50 bp, synthetic construct
  source 1..50: mol_type = other DNA
gtccaggggcgctccccccggggcccctgggccccccggccgccgccgcg

SEQ ID NO: 137 - DNA, 50 bp, synthetic construct
  source 1..50: mol_type = other DNA
gccgcccccctcttcccagccccgccgtccccggcgcgccgccggcggcc

SEQ ID NO: 138 - DNA, 50 bp, synthetic construct
  source 1..50: mol_type = other DNA
agagagagagagagagggggcgaggggcaggaggggggaggggccccctc

SEQ ID NO: 139 - DNA, 50 bp, synthetic construct
  source 1..50: mol_type = other DNA
ggggggagcgccgggagggggcggggtgggcgcgcgcggcgagcgaggga

SEQ ID NO: 140 - DNA, 50 bp, synthetic construct
  source 1..50: mol_type = other DNA
gcgcttcgccccgacccgcgcgcggcgccgacctgctccgcgcgcggggg

SEQ ID NO: 141 - DNA, 50 bp, synthetic construct
  source 1..50: mol_type = other DNA
ccgttcggcctaggcattgaagccctattcctaaccgagattcccgaccc

SEQ ID NO: 142 - DNA, 50 bp, synthetic construct
  source 1..50: mol_type = other DNA
taggcgtcgtccagaggttccacttgtcggagaccgtacaaccttgttac

SEQ ID NO: 143 - DNA, 50 bp, synthetic construct
  source 1..50: mol_type = other DNA
ctcgagagcgaccgggaacttttaggccccctctcccacatttagagcgc

SEQ ID NO: 144 - DNA, 50 bp, synthetic construct
  source 1..50: mol_type = other DNA
ttacccaagcggggctctctccccgggcacggaacctttcgcagcgccaa

SEQ ID NO: 145 - DNA, 50 bp, synthetic construct
  source 1..50: mol_type = other DNA
ggcctcttcggccgccctcggggcccctctcaagagaaaagaaacacttc

SEQ ID NO: 146 - DNA, 50 bp, synthetic construct
  source 1..50: mol_type = other DNA
taggggcttaggcctcaccgcctctacccgcggcgctccgcaggtcacgc

SEQ ID NO: 147 - DNA, 50 bp, synthetic construct
  source 1..50: mol_type = other DNA
ggcaaggcttccctgcccgctaccggaggcaacgggagccggctagcttt

SEQ ID NO: 148 - DNA, 50 bp, synthetic construct
  source 1..50: mol_type = other DNA
cctcttcccaaggtacacttgtcgtcaacttgtacccagtcagccaggac

SEQ ID NO: 149 - DNA, 50 bp, synthetic construct
  source 1..50: mol_type = other DNA
ggcggcgtccacgtctagaaccaccatcatcgtttataagtttgctcttg

SEQ ID NO: 150 - DNA, 50 bp, synthetic construct
  source 1..50: mol_type = other DNA
gcggcgctgctcatcctcccggcgacgccactcggaacttcggatcccgc

SEQ ID NO: 151 - DNA, 50 bp, synthetic construct
  source 1..50: mol_type = other DNA
gggtgcggaggaggggaggagggcgggtgcggggcgaggggcgggggcct

SEQ ID NO: 152 - DNA, 50 bp, synthetic construct
  source 1..50: mol_type = other DNA
cacacgcagcctcccgccgccgccgccgccgccgcccccacaccccagga

SEQ ID NO: 153 - DNA, 50 bp, synthetic construct
  source 1..50: mol_type = other DNA
gccggcagcggccgtcagctctcacctgccctcgccgcccccgccgcgcg

SEQ ID NO: 154 - DNA, 50 bp, synthetic construct
  source 1..50: mol_type = other DNA
ttgttgagtggacggcttagttgatcgggacttttacctaccgcgacctc

SEQ ID NO: 155 - DNA, 50 bp, synthetic construct
  source 1..50: mol_type = other DNA
tccacaaccaactatatctgtcgtcctgccaccggtaccttcagccttag

SEQ ID NO: 156 - DNA, 50 bp, synthetic construct
  source 1..50: mol_type = other DNA
gcgacgccctacttggcttgcggcccaattccgcgggctacggctgcgag

SEQ ID NO: 157 - DNA, 50 bp, synthetic construct
  source 1..50: mol_type = other DNA
gcgaccgcacctcggcccgcaccttacgctcacggatcacccggtgaaaa

SEQ ID NO: 158 - DNA, 50 bp, synthetic construct
  source 1..50: mol_type = other DNA
tctccagaaccccggctttgctagagttggataagagtttgaaatttacc

SEQ ID NO: 159 - DNA, 50 bp, synthetic construct
  source 1..50: mol_type = other DNA
tcgaccgcgagagcgtctgggctgcgtgggggcggtgcgtcaaaataggc

SEQ ID NO: 160 - DNA, 50 bp, synthetic construct
  source 1..50: mol_type = other DNA
catatccccgctttctgattagcttggtagatcatcgaccaagggaggct

SEQ ID NO: 161 - DNA, 50 bp, synthetic construct
  source 1..50: mol_type = other DNA
tcggtctcctttgagaccacctccaggcatcgccaggactgcacgtttag

SEQ ID NO: 162 - DNA, 50 bp, synthetic construct
  source 1..50: mol_type = other DNA
cctccacctcgtgctcgcgtgcacaatcctgggctttctaccacttgata

SEQ ID NO: 163 - DNA, 50 bp, synthetic construct
  source 1..50: mol_type = other DNA
ccctagggctccggagaggtcaggcggctcccgcgtggtggccgggcaga

SEQ ID NO: 164 - DNA, 50 bp, synthetic construct
  source 1..50: mol_type = other DNA
ccccgagcgtgctttcggcggcaccgcgttacttccacttccggccgcgc

SEQ ID NO: 165 - DNA, 50 bp, synthetic construct
  source 1..50: mol_type = other DNA
tgcagccgatgggtgggctgggcagaactttgtgcctggttcctcagatt

SEQ ID NO: 166 - DNA, 50 bp, synthetic construct
  source 1..50: mol_type = other DNA
gagccccggtgcgcgcgcagggggcttctccccctgccgcctcgctcgcg

SEQ ID NO: 167 - DNA, 50 bp, synthetic construct
  source 1..50: mol_type = other DNA
cccgccccgcctgacaggggtcacgcggggcccgcccagcgcggcagccc

SEQ ID NO: 168 - DNA, 50 bp, synthetic construct
  source 1..50: mol_type = other DNA
gagagggtggggaggaggggcgcgggggcggggccgctgccccccccacg

SEQ ID NO: 169 - DNA, 50 bp, synthetic construct
  source 1..50: mol_type = other DNA
ggggcgcccccgcgcggccgcagaggagcacccccccggcccggtgggga

SEQ ID NO: 170 - DNA, 50 bp, synthetic construct
  source 1..50: mol_type = other DNA
gcagcggcgcgagaggggggagggccgcgggtgggggcgccccttagggg

SEQ ID NO: 171 - DNA, 50 bp, synthetic construct
  source 1..50: mol_type = other DNA
ggggctcacaatgtcgggggggccgtcgtcgtgagcggcttagggccccg

SEQ ID NO: 172 - DNA, 50 bp, synthetic construct
  source 1..50: mol_type = other DNA
cgcccagccccgccgccgccgccgccgccaccgccgccgccgcccccgcc

SEQ ID NO: 173 - DNA, 50 bp, synthetic construct
  source 1..50: mol_type = other DNA
caggcaggcaggaggaggagggggcagaggcggggggccggggcgcagga

SEQ ID NO: 174 - DNA, 50 bp, synthetic construct
  source 1..50: mol_type = other DNA
cggcgctggccgaggccctgccgacccttccgggccgccccttccaccga

SEQ ID NO: 175 - DNA, 50 bp, synthetic construct
  source 1..50: mol_type = other DNA
gccgccccctggcagggggctggccgctggccggcggcggcccgcgtaaa

SEQ ID NO: 176 - DNA, 50 bp, synthetic construct
  source 1..50: mol_type = other DNA
gaggaggggcctcccccgcccgaggccgcccacgcccccacccgcccgcc

SEQ ID NO: 177 - DNA, 50 bp, synthetic construct
  source 1..50: mol_type = other DNA
agaaagggcggggggcaaggagggctggggaggtgggcgggagggaaggg

SEQ ID NO: 178 - DNA, 50 bp, synthetic construct
  source 1..50: mol_type = other DNA
aggcgcgtcaggcgggcctcctaagttgggccgccgcccaggccggcaca

SEQ ID NO: 179 - DNA, 50 bp, synthetic construct
  source 1..50: mol_type = other DNA
aacttttcttgaaacttctctctcaagttctcccgcactttggcaattct

SEQ ID NO: 180 - DNA, 50 bp, synthetic construct
  source 1..50: mol_type = other DNA
catttgaggtagattccgatttatggccgtgctctggctatcagttgttc

SEQ ID NO: 181 - DNA, 50 bp, synthetic construct
  source 1..50: mol_type = other DNA
cgccggccgcgcgcgggcccagaagggcctcagcccaacgaacccttacg

SEQ ID NO: 182 - DNA, 50 bp, synthetic construct
  source 1..50: mol_type = other DNA
cggcgagcaccccccgggttcaggaagactagctccgggtcgggcacctg

SEQ ID NO: 183 - DNA, 50 bp, synthetic construct
  source 1..50: mol_type = other DNA
ggtcgcggcttaggggcggggcgccccgcgccctgtacaccgcatgcctt

SEQ ID NO: 184 - DNA, 50 bp, synthetic construct
  source 1..50: mol_type = other DNA
atcagtcgcctccttttctttgattggtcctaagggagtcattgccgctc

SEQ ID NO: 185 - DNA, 50 bp, synthetic construct
  source 1..50: mol_type = other DNA
gccgggcagggggaggctctgcgctggagtctagtctgcaccgctgggcg

SEQ ID NO: 186 - DNA, 50 bp, synthetic construct
  source 1..50: mol_type = other DNA
tgaacgccggggcccaaggagggccccgatgcggacagactcgcagcgaa

SEQ ID NO: 187 - DNA, 50 bp, synthetic construct
  source 1..50: mol_type = other DNA
atcgccacctagtgagccgagcacgcagctacttcttgcgtcgatcgacg

SEQ ID NO: 188 - DNA, 50 bp, synthetic construct
  source 1..50: mol_type = other DNA
aatcatgaacctaccctctggcggacccttatggcccacgacatccgaaa

SEQ ID NO: 189 - DNA, 50 bp, synthetic construct
  source 1..50: mol_type = other DNA
cagatgccggtatggtgggacttgcgcgggctagagcagactagagcctt

SEQ ID NO: 190 - DNA, 50 bp, synthetic construct
  source 1..50: mol_type = other DNA
aaggacgtgggcatgggggcaccagaaacttatttcagactcacccgccg

SEQ ID NO: 191 - DNA, 50 bp, synthetic construct
  source 1..50: mol_type = other DNA
caattcgacctcggagccaccggtacgaagaacggggaacccggaggggg

SEQ ID NO: 192 - DNA, 50 bp, synthetic construct
  source 1..50: mol_type = other DNA
acgccacgtgcggagggacctgttcaaggaccgaagacactcgtggcacg

SEQ ID NO: 193 - DNA, 50 bp, synthetic construct
  source 1..50: mol_type = other DNA
aagttcgaggattcggtgacggacgaccactgggaccggcgggtggaggg

SEQ ID NO: 194 - DNA, 50 bp, synthetic construct
  source 1..50: mol_type = other DNA
acgggttgcgcgacaggcgggactcgctggacgtgcgcgtgttcgaagcc

SEQ ID NO: 195 - DNA, 50 bp, synthetic construct
  source 1..50: mol_type = other DNA
ccaattcccggtgccgttcttccaccggctgcgcgactggttgcggcacc

SEQ ID NO: 196 - DNA, 50 bp, synthetic construct
  source 1..50: mol_type = other DNA
aaggacaggaaggggtggtggttctggatgaagggcgtgaagctggactc

SEQ ID NO: 197 - DNA, 50 bp, synthetic construct
  source 1..50: mol_type = other DNA
tccggcggaccccattccagccgcgcgtgcgaccgctcataccacgcctc

SEQ ID NO: 198 - DNA, 50 bp, synthetic construct
  source 1..50: mol_type = other DNA
gaccaggggtgtctgagtctctcttgggtggtaccacgacagaggacggc

SEQ ID NO: 199 - DNA, 50 bp, synthetic construct
  source 1..50: mol_type = other DNA
catcggcaaggaggacgggcgacccggagggttgcccgggaggaggggag

SEQ ID NO: 200 - DNA, 50 bp, synthetic construct
  source 1..50: mol_type = other DNA
acctgttcaaggaccgaagacactcgtggcacgactggaggtttatggca

SEQ ID NO: 201 - DNA, 50 bp, synthetic construct
  source 1..50: mol_type = other DNA
gacggacgaccactgggaccggcgggtggaggggcggctcaagtggggac

SEQ ID NO: 202 - DNA, 50 bp, synthetic construct
  source 1..50: mol_type = other DNA
cgggactcgctggacgtgcgcgtgttcgaagcccacctgggccagttgaa

SEQ ID NO: 203 - DNA, 50 bp, synthetic construct
  source 1..50: mol_type = other DNA
tcttccaccggctgcgcgactggttgcggcaccgcgtgcacctgctgtac

SEQ ID NO: 204 - DNA, 50 bp, synthetic construct
  source 1..50: mol_type = other DNA
gtggttctggatgaagggcgtgaagctggactcggtgccgagacgggtcc

SEQ ID NO: 205 - DNA, 50 bp, synthetic construct
  source 1..50: mol_type = other DNA
cagccgcgcgtgcgaccgctcataccacgcctccgggacctctcctacaa

SEQ ID NO: 206 - DNA, 50 bp, synthetic construct
  source 1..50: mol_type = other DNA
tctctcttgggtggtaccacgacagaggacggctgttctggttgcagttc

SEQ ID NO: 207 - DNA, 50 bp, synthetic construct
  source 1..50: mol_type = other DNA
ccggaactcgtagacctaagacggattattttttgtaaataaaagtaacg

SEQ ID NO: 208 - DNA, 50 bp, synthetic construct
  source 1..50: mol_type = other DNA
ggttaaagataatttccaaggaaacaagggattcaggttgatgatttgac

SEQ ID NO: 209 - DNA, 50 bp, synthetic construct
  source 1..50: mol_type = other DNA
tttcaccaccgaccacaccgattacgggaccgggtgttcatagtgattcg

SEQ ID NO: 210 - DNA, 50 bp, synthetic construct
  source 1..50: mol_type = other DNA
accagacacacgaccgggtagtgaaaccgtttcttaagtggggtggtcac

SEQ ID NO: 211 - DNA, 50 bp, synthetic construct
  source 1..50: mol_type = other DNA
ctcactcgacgtgacactgttcgacgtgcacctaggactcttgaagtccg

SEQ ID NO: 212 - DNA, 50 bp, synthetic construct
  source 1..50: mol_type = other DNA
cacgagccacggaaatcactaccggaccgagtggacctgttggagttccc

SEQ ID NO: 213 - DNA, 50 bp, synthetic construct
  source 1..50: mol_type = other DNA
aacccctagacaggtgaggactacgacaatacccgttgggattccacttc

SEQ ID NO: 214 - DNA, 50 bp, synthetic construct
  source 1..50: mol_type = other DNA
accaccactccgggacccgtccgacgaccaccagatgggaacctgggtct

SEQ ID NO: 215 - DNA, 50 bp, synthetic construct
  source 1..50: mol_type = other DNA
gtagactgaggactcctcttcagacggcaatgacgggacaccccgttcca

SEQ ID NO: 216 - DNA, 50 bp, synthetic construct
  source 1..50: mol_type = other DNA
aaataagacgttcgttatgtttattatttagataagacgactctctagtg

SEQ ID NO: 217 - DNA, 50 bp, synthetic construct
  source 1..50: mol_type = other DNA
gtcacgggacaggaggtctatggtgactcgagtgacgggtactaagtctc

SEQ ID NO: 218 - DNA, 50 bp, synthetic construct
  source 1..50: mol_type = other DNA
gtaaagccgtttcttaagtggggactccacgtccgaaggaccgtcttcta

SEQ ID NO: 219 - DNA, 50 bp, synthetic construct
  source 1..50: mol_type = other DNA
tcgacgtacacctaggactcttgaagttcgaggaccctttacacgaccac

SEQ ID NO: 220 - DNA, 50 bp, synthetic construct
  source 1..50: mol_type = other DNA
acggtgtttcgtggacctactagagttcccgtggaaacgggtcgactcac

SEQ ID NO: 221 - DNA, 50 bp, synthetic construct
  source 1..50: mol_type = other DNA
agacggtagtacccgttggggtttcagttccgtgtaccgttcttccacga

SEQ ID NO: 222 - DNA, 50 bp, synthetic construct
  source 1..50: mol_type = other DNA
ccgaggaccaacagatgggtacctgggtctccaagaaactgtcgaaaccg

SEQ ID NO: 223 - DNA, 50 bp, synthetic construct
  source 1..50: mol_type = other DNA
ccgatgatagtgttcggacaccccgttccacttacaccttctacgacctc

SEQ ID NO: 224 - DNA, 50 bp, synthetic construct
  source 1..50: mol_type = other DNA
ttgcagactccaatagttattcgaggatcaggtctgcggtacccagtaaa

SEQ ID NO: 225 - DNA, 50 bp, synthetic construct
  source 1..50: mol_type = other DNA
gaaataagacgttcgttagtttattatttagataagacgattctctagtg

SEQ ID NO: 226 - DNA, 50 bp, synthetic construct
  source 1..50: mol_type = other DNA
ggtcacgggacaggaggtctatggtgactcgagtgacgggtactacgtct

SEQ ID NO: 227 - DNA, 50 bp, synthetic construct
  source 1..50: mol_type = other DNA
ggtaaagccgtttcttaagtggggactccacgtccgaaggaccgtcttct

SEQ ID NO: 228 - DNA, 50 bp, synthetic construct
  source 1..50: mol_type = other DNA
ttcgacgtacacctaggactcttgaagttcgaggaccctttacacgacca

SEQ ID NO: 229 - DNA, 50 bp, synthetic construct
  source 1..50: mol_type = other DNA
tacggtatttcgtggacctactagagttcccgtggaaacgggtcgactca

SEQ ID NO: 230 - DNA, 50 bp, synthetic construct
  source 1..50: mol_type = other DNA
gagacggtagtacccgttggggtttcagttccgtgtaccgttcttccacg

SEQ ID NO: 231 - DNA, 50 bp, synthetic construct
  source 1..50: mol_type = other DNA
tccgaggaccaacagatgggtacctgggtctccaagaaactgtcgaaacc

SEQ ID NO: 232 - DNA, 50 bp, synthetic construct
  source 1..50: mol_type = other DNA
tccgatgatagtgttcggacaccccgttccacttacaccttctacgacct

SEQ ID NO: 233 - DNA, 50 bp, synthetic construct
  source 1..50: mol_type = other DNA
cttgcagactccaatagttattcgaggatcaggtctgcggtacccagtaa

SEQ ID NO: 234 - DNA, 50 bp, synthetic construct
  source 1..50: mol_type = other DNA
gctaccatcacaccccagaggggtacgctctcatcccttgacggtccgta

SEQ ID NO: 235 - DNA, 50 bp, synthetic construct
  source 1..50: mol_type = other DNA
ccgccggcatcgcgccaccagggtggactggggtacggcttgagtcttca

SEQ ID NO: 236 - DNA, 50 bp, synthetic construct
  source 1..50: mol_type = other DNA
tgaatggtgaaacactaagtactgaccccacttcagcattgttccattgg

SEQ ID NO: 237 - DNA, 50 bp, synthetic construct
  source 1..50: mol_type = other DNA
tgtggcgggcagtgtggtaccctcacccaacgttttcttcatccatcgaa

SEQ ID NO: 238 - DNA, 50 bp, synthetic construct
  source 1..50: mol_type = other DNA
cttcagccttagcgatcattagcacctagtcttacggtgccacttatgca

SEQ ID NO: 239 - DNA, 50 bp, synthetic construct
  source 1..50: mol_type = other DNA
cgctctcgttcgcctggagtatttcacgcagcatcaggcctaacctcaga

SEQ ID NO: 240 - DNA, 50 bp, synthetic construct
  source 1..50: mol_type = other DNA
tagtaccgggaatgctggtcccgatgtgtgcacgatgttaccgcgtatgt

SEQ ID NO: 241 - DNA, 50 bp, synthetic construct
  source 1..50: mol_type = other DNA
ggccggcccttgagtttcctctgacggtcactatttgacctccttccacc

SEQ ID NO: 242 - DNA, 50 bp, synthetic construct
  source 1..50: mol_type = other DNA
gcacaacactttacaacccaattcagggcgttgctcgcgttgggaatagg

SEQ ID NO: 243 - DNA, 50 bp, synthetic construct
  source 1..50: mol_type = other DNA
caaaagtctctactcttacacggaagcccttggcactctgtccacgacgt

SEQ ID NO: 244 - DNA, 50 bp, synthetic construct
  source 1..50: mol_type = other DNA
ccacctcgtacaccaaattaagctacgttgcgcttcttggaatggaccag

SEQ ID NO: 245 - DNA, 50 bp, synthetic construct
  source 1..50: mol_type = other DNA
gctggcggacccctcatgccggcgttccaattttgagtttacttaactgc

SEQ ID NO: 246 - DNA, 50 bp, synthetic construct
  source 1..50: mol_type = other DNA
gcatttgctacagctgaacctccaacacgggaactccgcaccgaaggcct

SEQ ID NO: 247 - DNA, 50 bp, synthetic construct
  source 1..50: mol_type = other DNA
cttctgactgcgagtcacgctttcgcacccctcgtttgtcctaatctatg

SEQ ID NO: 248 - DNA, 50 bp, synthetic construct
  source 1..50: mol_type = other DNA
gtccacatcgccactttacgcatctctagacctccttatggccaccgctt

SEQ ID NO: 249 - DNA, 50 bp, synthetic construct
  source 1..50: mol_type = other DNA
cccgagttggacccttgacgtagactatgaccgttcgaactcagagcatc

SEQ ID NO: 250 - DNA, 50 bp, synthetic construct
  source 1..50: mol_type = other DNA
caattagccttaatgacccgcatttcgcgtgcgtccgccaaacaattcag

SEQ ID NO: 251 - DNA, 50 bp, synthetic construct
  source 1..50: mol_type = other DNA
aatgggcgtcttcttcgtggccgattgaggcacggtcgtcggcgccatta

SEQ ID NO: 252 - DNA, 50 bp, synthetic construct
  source 1..50: mol_type = other DNA
aagcccaacatttcatgaaagtcgcccctccttccctcatttcaattatg

SEQ ID NO: 253 - DNA, 50 bp, synthetic construct
  source 1..50: mol_type = other DNA
gtcaccccttatgacgtgttacccgcgttcggactacgtcggtacggcgc

SEQ ID NO: 254 - DNA, 50 bp, synthetic construct
  source 1..50: mol_type = other DNA
gaccagactctcctactggtcggtgtgaccttgactctgtgccaggtctg

SEQ ID NO: 255 - DNA, 50 bp, synthetic construct
  source 1..50: mol_type = other DNA
gcctacacgggtctaccctaatcgatcatccaccccattgccgagtggat

SEQ ID NO: 256 - DNA, 50 bp, synthetic construct
  source 1..50: mol_type = other DNA
tttgccatcgattatggcgtattgcagcgttctggtttctccccctggaa

SEQ ID NO: 257 - DNA, 50 bp, synthetic construct
  source 1..50: mol_type = other DNA
gactgctcaccgcctgcccactcattacagaccctttgacggactacctc

SEQ ID NO: 258 - DNA, 50 bp, synthetic construct
  source 1..50: mol_type = other DNA
gtcgctacgcaactcgattggccatgattacttggcactccgaattggaa

SEQ ID NO: 259 - DNA, 50 bp, synthetic construct
  source 1..50: mol_type = other DNA
actgggaaattcccaggacttccttgcaacttctgctgctgcaactatcc

SEQ ID NO: 260 - DNA, 50 bp, synthetic construct
  source 1..50: mol_type = other DNA
tttacgccttctctattcacgactttcgtagattcgtgctttgaacgggg

SEQ ID NO: 261 - DNA, 50 bp, synthetic construct
  source 1..50: mol_type = other DNA
tctcctggcctcacctgcgtagtgaccacaagcccaacagtacggttacc

SEQ ID NO: 262 - DNA, 50 bp, synthetic construct
  source 1..50: mol_type = other DNA
cactctgtcaagccagggatagacggcacccgcgacctcttgactccccc

SEQ ID NO: 263 - DNA, 50 bp, synthetic construct
  source 1..50: mol_type = other DNA
ttcatccagggttcccataccgacaagcggtaaatttcaccatgcgctcg

SEQ ID NO: 264 - DNA, 50 bp, synthetic construct
  source 1..50: mol_type = other DNA
gggttctcaagtatagctgccgccacaaaccgtggagctacagccgagta

SEQ ID NO: 265 - DNA, 50 bp, synthetic construct
  source 1..50: mol_type = other DNA
caagacttaccttcccggtagcgagttgcctattttccatgaggccccta

SEQ ID NO: 266 - DNA, 50 bp, synthetic construct
  source 1..50: mol_type = other DNA
ttcggtcgaactgacgctcgcactgccgcgctcgtccacgctttcgtcca

SEQ ID NO: 267 - DNA, 50 bp, synthetic construct
  source 1..50: mol_type = other DNA
tcattgcctcctcgtgcttccaaccgattaggaccagcctgtagtcctcc

SEQ ID NO: 268 - DNA, 50 bp, synthetic construct
  source 1..50: mol_type = other DNA
tgggcattaggcccaacgcctgtcacagaccacccatcaaactgaccccg

SEQ ID NO: 269 - DNA, 50 bp, synthetic construct
  source 1..50: mol_type = other DNA
cctgcggtcagacgtacctcggctggaactttatggtgggaaattacaaa

SEQ ID NO: 270 - DNA, 50 bp, synthetic construct
  source 1..50: mol_type = other DNA
aatgatatcgaactgtgacttgtaactcggaactacacatcctatccacc

SEQ ID NO: 271 - DNA, 50 bp, synthetic construct
  source 1..50: mol_type = other DNA
cactttaacttgagcgacacttctacgtcacatgggcgccgttctgcctt

SEQ ID NO: 272 - DNA, 50 bp, synthetic construct
  source 1..50: mol_type = other DNA
cagcccattcaaggctggacgtgcttaccgcattactaccggtccgacag

SEQ ID NO: 273 - DNA, 50 bp, synthetic construct
  source 1..50: mol_type = other DNA
agaactagcttcggggccatttgccgccggcattgatattgccaggattc

SEQ ID NO: 274 - DNA, 50 bp, synthetic construct
  source 1..50: mol_type = other DNA
tgcatatgccacactgcggacgggccacggccttccaattaactacccca

SEQ ID NO: 275 - DNA, 50 bp, synthetic construct
  source 1..50: mol_type = other DNA
cagcttctatggtcgaccgacgttgacaaataatttttgtgtcgtgacac

SEQ ID NO: 276 - DNA, 50 bp, synthetic construct
  source 1..50: mol_type = other DNA
ggcattgaagccctcttccgtgcgactatacatccactccagggagcgcc

SEQ ID NO: 277 - DNA, 50 bp, synthetic construct
  source 1..50: mol_type = other DNA
caccagtccatctcttatggttccgcgaactctcttgagcccacttcctt

SEQ ID NO: 278 - DNA, 50 bp, synthetic construct
  source 1..50: mol_type = other DNA
cgggacgaaggtccttttcggagattcgtagtccattgtagtttagcatg

SEQ ID NO: 279 - DNA, 50 bp, synthetic construct
  source 1..50: mol_type = other DNA
tccgtttaggccttttagttccgactccgcactactgctccgtgatgcca

SEQ ID NO: 280 - DNA, 50 bp, synthetic construct
  source 1..50: mol_type = other DNA
cctgcctcttccgatacaaccggcccgctgccaacagggccaaattcgca

SEQ ID NO: 281 - DNA, 50 bp, synthetic construct
  source 1..50: mol_type = other DNA
ccggctttccgcatcagctaccctttgtccaattataaggacatgaacca

SEQ ID NO: 282 - DNA, 50 bp, synthetic construct
  source 1..50: mol_type = other DNA
gcggccttctggttcccaaggacaggttgcaattagccccgtcccactca

SEQ ID NO: 283 - DNA, 50 bp, synthetic construct
  source 1..50: mol_type = other DNA
tacgacctccatagtcttcacgcttacgactgtattcattgctatttcgc

SEQ ID NO: 284 - DNA, 50 bp, synthetic construct
  source 1..50: mol_type = other DNA
tcgctgcgaatacgcaacaacccatcccctcgcaagacattcggacgctt

SEQ ID NO: 285 - DNA, 50 bp, synthetic construct
  source 1..50: mol_type = other DNA
gagtgaccagctcagccggacgcgccttctacattgccccgatttggtac

SEQ ID NO: 286 - DNA, 50 bp, synthetic construct
  source 1..50: mol_type = other DNA
acccttccgggtctgtcggtcctacaaccgaatcttcgtcggtagtaaat

SEQ ID NO: 287 - DNA, 50 bp, synthetic construct
  source 1..50: mol_type = other DNA
acttctccctttgttgggtctggcggtcgattccagggtttcagtaccaa

SEQ ID NO: 288 - DNA, 50 bp, synthetic construct
  source 1..50: mol_type = other DNA
tacgtttgacgcttatggcctcttacaatagtgccctctgtgtgccgccc

SEQ ID NO: 289 - DNA, 50 bp, synthetic construct
  source 1..50: mol_type = other DNA
gcacttaagtagaggcccccatctcgtgacaaagccgttcccccagtagg

SEQ ID NO: 290 - DNA, 50 bp, synthetic construct
  source 1..50: mol_type = other DNA
ccccactttccggttagtttggccctctatcgaccaagaggggctttcga

SEQ ID NO: 291 - DNA, 50 bp, synthetic construct
  source 1..50: mol_type = other DNA
acccattgtgattgacctcctggcttggctgattacaactttttaatcgc

SEQ ID NO: 292 - DNA, 50 bp, synthetic construct
  source 1..50: mol_type = other DNA
gacccgcaattcaacgtcccatatctgggctttgggccactagatcggta

SEQ ID NO: 293 - DNA, 50 bp, synthetic construct
  source 1..50: mol_type = other DNA
agtcgctgaatataagacatcgttccaattggcttatcccctcggcttcc

SEQ ID NO: 294 - DNA, 50 bp, synthetic construct
  source 1..50: mol_type = other DNA
tggcacatgcatgttcgtcaccctcgtgcgaatccgcacactgacgcatg

SEQ ID NO: 295 - DNA, 50 bp, synthetic construct
  source 1..50: mol_type = other DNA
cacttggtcatggcactccctttccgcttttcttggggccgctcccctca

SEQ ID NO: 296 - DNA, 50 bp, synthetic construct
  source 1..50: mol_type = other DNA
gcaccataggacagacttatacccccctggtaggaggttccgatttatga

SEQ ID NO: 297 - DNA, 50 bp, synthetic construct
  source 1..50: mol_type = other DNA
tatgtcccactgtcggggcatgtgtttttacgtgtacgacactcgagcta

SEQ ID NO: 298 - DNA, 50 bp, synthetic construct
  source 1..50: mol_type = other DNA
ttgcccctcgtcgggtctcggacttagtcacacacacaatcaccttcgca

SEQ ID NO: 299 - DNA, 50 bp, synthetic construct
  source 1..50: mol_type = other DNA
ttgactttgtagattcatggggctccttttctttagttggctctaagggg

SEQ ID NO: 300 - DNA, 50 bp, synthetic construct
  source 1..50: mol_type = other DNA
tttgggtcacacaaagctgtgtgatagtaattgacttaggtatccaatta

SEQ ID NO: 301 - DNA, 50 bp, synthetic construct
  source 1..50: mol_type = other DNA
cgattagacgctattcgcagccattccactatacttggcaatattggccg

SEQ ID NO: 302 - DNA, 50 bp, synthetic construct
  source 1..50: mol_type = other DNA
cggctaccatcagcccccaaagggggacactctcatcctgcggcggttcg

SEQ ID NO: 303 - DNA, 50 bp, synthetic construct
  source 1..50: mol_type = other DNA
aaccaccgctatcgcttctccagtgtgggcaagggtatggcttgtgcctt

SEQ ID NO: 304 - DNA, 50 bp, synthetic construct
  source 1..50: mol_type = other DNA
gcattgttccatcggcatagccttccacgccgacctagtggaggaaagat

SEQ ID NO: 305 - DNA, 50 bp, synthetic construct
  source 1..50: mol_type = other DNA
cttcagccactccattggaaaatcctcggtcggcggcttccaccctgtct

SEQ ID NO: 306 - DNA, 50 bp, synthetic construct
  source 1..50: mol_type = other DNA
gcgccacttatgcaagggcccggaacatgtgtggcgggcagtgtggtgct

SEQ ID NO: 307 - DNA, 50 bp, synthetic construct
  source 1..50: mol_type = other DNA
agcctagcgtcagacgttgagctgacgcacttcgaccttagcgatcatta

SEQ ID NO: 308 - DNA, 50 bp, synthetic construct
  source 1..50: mol_type = other DNA
gttacctgtcttgtttcccgtcgctttggcgctccaattcggttagggtg

SEQ ID NO: 309 - DNA, 50 bp, synthetic construct
  source 1..50: mol_type = other DNA
ggcctccttccacccctactgcagtttagtagtacggggaatactggacc

SEQ ID NO: 310 - DNA, 50 bp, synthetic construct
  source 1..50: mol_type = other DNA
cgcgttgggaactagaatcaacggtcgtaagtcaacccgtgagattccac

SEQ ID NO: 311 - DNA, 50 bp, synthetic construct
  source 1..50: mol_type = other DNA
cactgtccaccacgtaccaacagcagtcgagcacagcactctacaaccca

SEQ ID NO: 312 - DNA, 50 bp, synthetic construct
  source 1..50: mol_type = other DNA
cttggaatggtccagaactgtaggagactgttaggatctctatcctgcag

SEQ ID NO: 313 - DNA, 50 bp, synthetic construct
  source 1..50: mol_type = other DNA
agtttccttaactgcccccgggcgtgttcgccacctcgtacaccaaatta

SEQ ID NO: 314 - DNA, 50 bp, synthetic construct
  source 1..50: mol_type = other DNA
ggggaatcacgacgtcgattgcgtaattcgtgaggcggacccctcatgcc

SEQ ID NO: 315 - DNA, 50 bp, synthetic construct
  source 1..50: mol_type = other DNA
tgtcctaatctatgggaccatcaggtgcggcatttgctactcacgattca

SEQ ID NO: 316 - DNA, 50 bp, synthetic construct
  source 1..50: mol_type = other DNA
gtggtcaccgcttccgctgagagaccagacattgactgcgactcctcgct

SEQ ID NO: 317 - DNA, 50 bp, synthetic construct
  source 1..50: mol_type = other DNA
aactcacgtcttctcctctcaccttaaggtgcacatcgccactttacgca

SEQ ID NO: 318 - DNA, 50 bp, synthetic construct
  source 1..50: mol_type = other DNA
ccaaagaattcagactacactttcgggggccgagttggcccctcccagta

SEQ ID NO: 319 - DNA, 50 bp, synthetic construct
  source 1..50: mol_type = other DNA
gtcggcgccattatgcatccaccgttcgcaacaggccttaataacccgca

SEQ ID NO: 320 - DNA, 50 bp, synthetic construct
  source 1..50: mol_type = other DNA
tggcaagcttatcccgccatggaactgccatggattggtctttcggtgcc

SEQ ID NO: 321 - DNA, 50 bp, synthetic construct
  source 1..50: mol_type = other DNA
tcgttgcggcgcactcactacttccaaaagcctagcatttcgagacaaca

SEQ ID NO: 322 - DNA, 50 bp, synthetic construct
  source 1..50: mol_type = other DNA
gtgccgggtctgaggatgccctccgtcgtcatcccttagaaggcgttacc

SEQ ID NO: 323 - DNA, 50 bp, synthetic construct
  source 1..50: mol_type = other DNA
tgccgagtggttccgctgctacgcatcggctggactctcccactagccgg

SEQ ID NO: 324 - DNA, 50 bp, synthetic construct
  source 1..50: mol_type = other DNA
tattttccaccgaagccgatggtgaatgtctacctgggcgccgcgtaatc

SEQ ID NO: 325 - DNA, 50 bp, synthetic construct
  source 1..50: mol_type = other DNA
ctgaccctattgaggccctttggccccgattatggcctaccaacaaactt

SEQ ID NO: 326 - DNA, 50 bp, synthetic construct
  source 1..50: mol_type = other DNA
taccctcgaacgagggactacaatcgccgcctgcccactcattgtgcacc

SEQ ID NO: 327 - DNA, 50 bp, synthetic construct
  source 1..50: mol_type = other DNA
ctcaaactaggaccgagtcctgcttgcgaccgccgcacggattatgtacg

SEQ ID NO: 328 - DNA, 50 bp, synthetic construct
  source 1..50: mol_type = other DNA
acaccgctgtgtacctcgactgtctatgattagctagctcctgaattggt

SEQ ID NO: 329 - DNA, 50 bp, synthetic construct
  source 1..50: mol_type = other DNA
aaagggtaaggcgttccttcattctagggactttctactagtccaactat

SEQ ID NO: 330 - DNA, 50 bp, synthetic construct
  source 1..50: mol_type = other DNA
catcgatacacgcctgccctattcacgactttcgtagattcgtacttcgg

SEQ ID NO: 331 - DNA, 50 bp, synthetic construct
  source 1..50: mol_type = other DNA
tcatgctctcctggccctacctgcgtggcgaccacatggtcaacaagacg

SEQ ID NO: 332 - DNA, 50 bp, synthetic construct
  source 1..50: mol_type = other DNA
ttgcagcactctgtcaagccagggataggcagcgcccgcgtcctttaaac

SEQ ID NO: 333 - DNA, 50 bp, synthetic construct
  source 1..50: mol_type = other DNA
cccgacatcagtcagggttcccaacccgacaagcgggtaatttcgccatg

SEQ ID NO: 334 - DNA, 50 bp, synthetic construct
  source 1..50: mol_type = other DNA
tagagggggttctcaggtgtagctgcccctccaaaccgtggagctacagc

SEQ ID NO: 335 - DNA, 50 bp, synthetic construct
  source 1..50: mol_type = other DNA
ggccaccaaggcgtaccttcccggtagcgagttgcctattttcgatgggg

SEQ ID NO: 336 - DNA, 50 bp, synthetic construct
  source 1..50: mol_type = other DNA
tccgtgttccctcgaactgacgctctggatgttcagctcgtccctgcttt

SEQ ID NO: 337 - DNA, 50 bp, synthetic construct
  source 1..50: mol_type = other DNA
gattttccattgcctccgcgggtttccaagggagtcttaccaacctttag

SEQ ID NO: 338 - DNA, 50 bp, synthetic construct
  source 1..50: mol_type = other DNA
tgggcggcgggaatagcccgcccctctgtcacagtccacccgtcaaactg

SEQ ID NO: 339 - DNA, 50 bp, synthetic construct
  source 1..50: mol_type = other DNA
tttggcctcgcggtcgaagccacctccgtagccaccctatgatgggaccg

SEQ ID NO: 340 - DNA, 50 bp, synthetic construct
  source 1..50: mol_type = other DNA
cctcgaaatgacgtcggactataacttacaaccatgtcgaacatgtccta

SEQ ID NO: 341 - DNA, 50 bp, synthetic construct
  source 1..50: mol_type = other DNA
ctgagccactttaatatcatggacacttctacgtccaatgggcgctgtcc

SEQ ID NO: 342 - DNA, 50 bp, synthetic construct
  source 1..50: mol_type = other DNA
aaggaacagcccattcaaggctgggcgtgctttccgcgttgctagacccg

SEQ ID NO: 343 - DNA, 50 bp, synthetic construct
  source 1..50: mol_type = other DNA
gcttccacgcttaacttcggggtcatttgccgccggcattgatattgcca

SEQ ID NO: 344 - DNA, 50 bp, synthetic construct
  source 1..50: mol_type = other DNA
ttccgcttcatatccccgactgcggacgggccacgaccttccaattctcc

SEQ ID NO: 345 - DNA, 50 bp, synthetic construct
  source 1..50: mol_type = other DNA
ctctctcggcgtcacttatccgggtccgctgacaaatcgtttttgtgtcc

SEQ ID NO: 346 - DNA, 50 bp, synthetic construct
  source 1..50: mol_type = other DNA
attccttgagccgttttactggggcattgaagccctcttccccacgagac

SEQ ID NO: 347 - DNA, 50 bp, synthetic construct
  source 1..50: mol_type = other DNA
gggcatggcgtttggctgtgtccatccgctcctctcttaggattccacta

SEQ ID NO: 348 - DNA, 50 bp, synthetic construct
  source 1..50: mol_type = other DNA
ctttatatcatcgcttcaaggactaaggtgtgacggttcttttcggagat

SEQ ID NO: 349 - DNA, 50 bp, synthetic construct
  source 1..50: mol_type = other DNA
aggttcgtcaatccgaccctttatccgtttaggcaaagggtattccgact

SEQ ID NO: 350 - DNA, 50 bp, synthetic construct
  source 1..50: mol_type = other DNA
aggagtggtaaactcgttacccccctgcgtcctcctatcccattcgcgcc

SEQ ID NO: 351 - DNA, 50 bp, synthetic construct
  source 1..50: mol_type = other DNA
agccctggattcggctccggctttccgcatccgctacctgttgtccaact

SEQ ID NO: 352 - DNA, 50 bp, synthetic construct
  source 1..50: mol_type = other DNA
cccactcttaggggaggtggcttacggattccaaaggactccttccgagc

SEQ ID NO: 353 - DNA, 50 bp, synthetic construct
  source 1..50: mol_type = other DNA
ttcggtctggccttcctgaccacctcgcgaatcttcactcttacggccat

SEQ ID NO: 354 - DNA, 50 bp, synthetic construct
  source 1..50: mol_type = other DNA
tttgcatagtggcttcgacgcctgacaagaagcttgtcaccatcctctcg

SEQ ID NO: 355 - DNA, 50 bp, synthetic construct
  source 1..50: mol_type = other DNA
tggtaaatttctcacgcattatcgagtgaccagctcactgagacgcggct

SEQ ID NO: 356 - DNA, 50 bp, synthetic construct
  source 1..50: mol_type = other DNA
atatgcaattcaccttttcctacacctcaacgaatctgttggtcctacaa

SEQ ID NO: 357 - DNA, 50 bp, synthetic construct
  source 1..50: mol_type = other DNA
tgacgctcactattctaggcatcagctttccctttgtcgggtctggcggt

SEQ ID NO: 358 - DNA, 50 bp, synthetic construct
  source 1..50: mol_type = other DNA
ccggggatggcccaatggcttaagtcagtttgaggcttacggttactgaa

SEQ ID NO: 359 - DNA, 50 bp, synthetic construct
  source 1..50: mol_type = other DNA
ggctttatcgaaatcccgatcggagttccattctcagaacctccatctcg

SEQ ID NO: 360 - DNA, 50 bp, synthetic construct
  source 1..50: mol_type = other DNA
ttcacgcccctactccacacccatccccactttacggttagcttggacct

SEQ ID NO: 361 - DNA, 50 bp, synthetic construct
  source 1..50: mol_type = other DNA
agatgggtacaggtcccacttcaagtccattgtgacttacctccgggctt

SEQ ID NO: 362 - DNA, 50 bp, synthetic construct
  source 1..50: mol_type = other DNA
cggcgtcgctttcgctcagacttatcccgcgtactcatgcaccagcatct

SEQ ID NO: 363 - DNA, 50 bp, synthetic construct
  source 1..50: mol_type = other DNA
cgcacggaaaacatcttacttggccgctcaatgctagggcacgttccaat

SEQ ID NO: 364 - DNA, 50 bp, synthetic construct
  source 1..50: mol_type = other DNA
tcccctcactttctctaggactttggcacacggatgttcatcagtctcgg

SEQ ID NO: 365 - DNA, 50 bp, synthetic construct
  source 1..50: mol_type = other DNA
gatttatgagggatcactggctatcacttggtcatggcactccctttcca

SEQ ID NO: 366 - DNA, 50 bp, synthetic construct
  source 1..50: mol_type = other DNA
cacctaggactcatgccgccttgtgcactttaaggcagccttaggccctc

SEQ ID NO: 367 - DNA, 50 bp, synthetic construct
  source 1..50: mol_type = other DNA
tctccagacctttccgggcggtatcctccattgtcgggacatcagttttg

SEQ ID NO: 368 - DNA, 50 bp, synthetic construct
  source 1..50: mol_type = other DNA
ttctccgaacggagaaccccaacatcctgtgagacatgcctcaatgtttc

SEQ ID NO: 369 - DNA, 50 bp, synthetic construct
  source 1..50: mol_type = other DNA
gcctccttctctttcgtttacgctaagggactcatcgccgctcgctttgc

SEQ ID NO: 370 - DNA, 50 bp, synthetic construct
  source 1..50: mol_type = other DNA
cataggtatagacttaagtatcctatactcttccgtctgggccccttgac

SEQ ID NO: 371 - DNA, 50 bp, synthetic construct
  source 1..50: mol_type = other DNA
ctcgacattcgttcgaaactaggcctctaaaggcttacccctttgggtgg

SEQ ID NO: 372 - DNA, 50 bp, synthetic construct
  source 1..50: mol_type = other DNA
gcgtgccacctacggaaccgtgatcctcggctacttcctgccctgcttgt

SEQ ID NO: 373 - DNA, 50 bp, synthetic construct
  source 1..50: mol_type = other DNA
ttttccctaacctactcctacattacaaaacaatataagcttagatcaaa

SEQ ID NO: 374 - DNA, 50 bp, synthetic construct
  source 1..50: mol_type = other DNA
cgcacagtgttagcgcgccacttatgcagggacgaggaacgtgtgtggcg

SEQ ID NO: 375 - DNA, 50 bp, synthetic construct
  source 1..50: mol_type = other DNA
gttgtgcgcccgatgttaccgactctgttacccaaggctgcggctttccg

SEQ ID NO: 376 - DNA, 50 bp, synthetic construct
  source 1..50: mol_type = other DNA
ggcggatactatttatcctccttcctcacctgctgccatccaggcatacg

SEQ ID NO: 377 - DNA, 50 bp, synthetic construct
  source 1..50: mol_type = other DNA
gctcgctctgggtgcgagaatcaatggtcgcctaggaaaaaacctacggc

SEQ ID NO: 378 - DNA, 50 bp, synthetic construct
  source 1..50: mol_type = other DNA
agttgcggcctgtagagtggtctccgctgtcgacatactatcggtccaac

SEQ ID NO: 379 - DNA, 50 bp, synthetic construct
  source 1..50: mol_type = other DNA
ctgactttgaatttccttaaccgccccctcgtggtgttgcgcacctcgga

SEQ ID NO: 380 - DNA, 50 bp, synthetic construct
  source 1..50: mol_type = other DNA
cgaaactcgacagggtcacggcttcccttcgacaattcaggcggcggacc

SEQ ID NO: 381 - DNA, 50 bp, synthetic construct
  source 1..50: mol_type = other DNA
cgcgcttggcctaatctatgggcccatcaggatcgacatttgctacgcct

SEQ ID NO: 382 - DNA, 50 bp, synthetic construct
  source 1..50: mol_type = other DNA
atcggccaaattattcagagaccactttaggacgtcgaattgacaccctt

SEQ ID NO: 383 - DNA, 50 bp, synthetic construct
  source 1..50: mol_type = other DNA
aagaattgccctaccgaaaagtaatcacatttctcgaaaaccttattctc

SEQ ID NO: 384 - DNA, 50 bp, synthetic construct
  source 1..50: mol_type = other DNA
ccccacgtcgtccgcgctttggaggcgttacactctttagcgctgccccc

SEQ ID NO: 385 - DNA, 50 bp, synthetic construct
  source 1..50: mol_type = other DNA
ctagccatgcccaacactctcgttctcgggcctctaccttggactctgtt

SEQ ID NO: 386 - DNA, 50 bp, synthetic construct
  source 1..50: mol_type = other DNA
aaagcggattcctacccagacgccggctaatccatcaaccaatccattac

SEQ ID NO: 387 - DNA, 50 bp, synthetic construct
  source 1..50: mol_type = other DNA
gaccctttgacccctattatgacctatccgttaataaggacattaccaaa

SEQ ID NO: 388 - DNA, 50 bp, synthetic construct
  source 1..50: mol_type = other DNA
tcaaatccgagcaccgcatgccgagtcattgtgcacctattggatgggaa

SEQ ID NO: 389 - DNA, 50 bp, synthetic construct
  source 1..50: mol_type = other DNA
accagctggccaacagactattccgtcacggcccgtcgatgcgcgatcca

SEQ ID NO: 390 - DNA, 50 bp, synthetic construct
  source 1..50: mol_type = other DNA
acctacacaatcatcagactccccttccaccgaggtcatgctctccttgc

SEQ ID NO: 391 - DNA, 50 bp, synthetic construct
  source 1..50: mol_type = other DNA
agcgggtaatttccccttgcactcgacccaaatctggcagcactctgtcc

SEQ ID NO: 392 - DNA, 50 bp, synthetic construct
  source 1..50: mol_type = other DNA
cgaacgatgaagctacagccaagaaaggtaggacccacacgtcgtcgtgg

SEQ ID NO: 393 - DNA, 50 bp, synthetic construct
  source 1..50: mol_type = other DNA
tactgtctttttgatggagcccctattgacccaccagcgtccgttctcgg

SEQ ID NO: 394 - DNA, 50 bp, synthetic construct
  source 1..50: mol_type = other DNA
gttctttaggtctccgctttcggcccggatcgcttggagtctcaggagga

SEQ ID NO: 395 - DNA, 50 bp, synthetic construct
  source 1..50: mol_type = other DNA
aagcaagaactctgtggtgaaagaatactgacacaaagattgaatgaaac

SEQ ID NO: 396 - DNA, 50 bp, synthetic construct
  source 1..50: mol_type = other DNA
ttcgaaccaatatccatacgtcacattcaccctcttaagctaatattcag

SEQ ID NO: 397 - DNA, 50 bp, synthetic construct
  source 1..50: mol_type = other DNA
cgtgttcggtctcagaaggtaacccttcgcttctggggcatctcaaaatg

SEQ ID NO: 398 - DNA, 50 bp, synthetic construct
  source 1..50: mol_type = other DNA
cgtacttaccatcttgctccaggggtgacagggatcgatcttgaatcact

SEQ ID NO: 399 - DNA, 50 bp, synthetic construct
  source 1..50: mol_type = other DNA
tttgccgcccccattgatattgggagaattccatcgctttacggaacagc

SEQ ID NO: 400 - DNA, 50 bp, synthetic construct
  source 1..50: mol_type = other DNA
tgtctcccgctgtggacgggtcacgaccgtgcacttcgacctcaagtcaa

SEQ ID NO: 401 - DNA, 50 bp, synthetic construct
  source 1..50: mol_type = other DNA
gtcactgatccccccgggctgacaaattatttttgtatcgagagacgatc

SEQ ID NO: 402 - DNA, 50 bp, synthetic construct
  source 1..50: mol_type = other DNA
tggctctaggcagtgtccacggggatcgactcatccgattccgcaaaatt

SEQ ID NO: 403 - DNA, 50 bp, synthetic construct
  source 1..50: mol_type = other DNA
catgaccaaattgactaagtacctcaggaacttttccctcaatcaaaaca

SEQ ID NO: 404 - DNA, 50 bp, synthetic construct
  source 1..50: mol_type = other DNA
caaattcggacccctctccacattatcgctcttggtccacatccgtaata

SEQ ID NO: 405 - DNA, 50 bp, synthetic construct
  source 1..50: mol_type = other DNA
actgcaactcccgattaaagactgtgaagctctatacaaaacatcctaac

SEQ ID NO: 406 - DNA, 50 bp, synthetic construct
  source 1..50: mol_type = other DNA
aaggaaacctggaacgtccatacacctaggaccatcatcatcgtcgtttc

SEQ ID NO: 407 - DNA, 50 bp, synthetic construct
  source 1..50: mol_type = other DNA
ccttgtggaaaaaaaaccactaacacatctcaccgctggacacacccaac

SEQ ID NO: 408 - DNA, 50 bp, synthetic construct
  source 1..50: mol_type = other DNA
gtctagtgggtagctccagtctccgtggattttacctgcccttaattagg

SEQ ID NO: 409 - DNA, 50 bp, synthetic construct
  source 1..50: mol_type = other DNA
ctcagaaaccgggttctgttacccttccacccgaatcttcgtcggtggga

SEQ ID NO: 410 - DNA, 50 bp, synthetic construct
  source 1..50: mol_type = other DNA
acacagggctttctctttgttgagtctgataccaattccggggatttatg

SEQ ID NO: 411 - DNA, 50 bp, synthetic construct
  source 1..50: mol_type = other DNA
gcatgacattttgaggcttgagcagtggcagcatcttccaacctcactcc

SEQ ID NO: 412 - DNA, 50 bp, synthetic construct
  source 1..50: mol_type = other DNA
cggactgacctccaaccaccgccccatctcgtgattaacctgcaaatccc

SEQ ID NO: 413 - DNA, 50 bp, synthetic construct
  source 1..50: mol_type = other DNA
attatcccggaccttagtgtcgtaactttgggcttcggccagctagataa

SEQ ID NO: 414 - DNA, 50 bp, synthetic construct
  source 1..50: mol_type = other DNA
tccaaaaataggaacatcagcatccctttggctatacgggcgtcgcaaca

SEQ ID NO: 415 - DNA, 50 bp, synthetic construct
  source 1..50: mol_type = other DNA
ctgatcacaacgtagcaggcagaactttgtgcccggtccctcaagaaaca

SEQ ID NO: 416 - DNA, 50 bp, synthetic construct
  source 1..50: mol_type = other DNA
acaccgcgcattccttacttaagaaggctttcttagtcaccattgtaaca

SEQ ID NO: 417 - DNA, 50 bp, synthetic construct
  source 1..50: mol_type = other DNA
ccttacgacttttcatggggatatccccccacttttcatggactttggtc

SEQ ID NO: 418 - DNA, 50 bp, synthetic construct
  source 1..50: mol_type = other DNA
acaccctccgtagttgaaggttaagatttatgcagagttctggctatctc

SEQ ID NO: 419 - DNA, 50 bp, synthetic construct
  source 1..50: mol_type = other DNA
gcaccctatttagcttaaaataaaccttaaaatcttggcatctcccacta

SEQ ID NO: 420 - DNA, 50 bp, synthetic construct
  source 1..50: mol_type = other DNA
tcttgtttgacttagggaaaatcattataatccctctacaccacatatct

SEQ ID NO: 421 - DNA, 50 bp, synthetic construct
  source 1..50: mol_type = other DNA
gaatcatccgcctccttttctttcgttatacgctacagcaatcattgccg

SEQ ID NO: 422 - DNA, 50 bp, synthetic construct
  source 1..50: mol_type = other DNA
ctgaaggatcgtgtaaaaaaaacacaactaggcacatcctaacccttggg

SEQ ID NO: 423 - DNA, 50 bp, synthetic construct
  source 1..50: mol_type = other DNA
acgccagtatcgtacccccaatatggaccagagcaaagctagggtcatca

SEQ ID NO: 424 - DNA, 50 bp, synthetic construct
  source 1..50: mol_type = other DNA
cgttgtgacaatcatactcattgttcttaaggttaagaggtccgtatgcg

SEQ ID NO: 425 - DNA, 50 bp, synthetic construct
  source 1..50: mol_type = other DNA
taattaaggtattaaatgtggttgaaggatttgaattttaacccaattag

SEQ ID NO: 426 - DNA, 50 bp, synthetic construct
  source 1..50: mol_type = other DNA
attggtaacatccggattttcgtcggtggttatttctttcgcaagttcga

SEQ ID NO: 427 - DNA, 50 bp, synthetic construct
  source 1..50: mol_type = other DNA
tttctccctgtcgagaagaccttgccttttttggaaattatcacttatta

SEQ ID NO: 428 - DNA, 50 bp, synthetic construct
  source 1..50: mol_type = other DNA
aattcaagttaaaatttgaacgatttttttgttgttttagtttttcattc

SEQ ID NO: 429 - DNA, 50 bp, synthetic construct
  source 1..50: mol_type = other DNA
tctaaaaatccatctccacttttcggattgctcgaaccactatcgaccaa

SEQ ID NO: 430 - DNA, 50 bp, synthetic construct
  source 1..50: mol_type = other DNA
ttcttggggcggacaaatggtttttgtagtggagatcgtaatgttcataa

SEQ ID NO: 431 - DNA, 50 bp, synthetic construct
  source 1..50: mol_type = other DNA
agacaattgggttgtggccttacggatttcctttctaggtttttctattt

SEQ ID NO: 432 - DNA, 50 bp, synthetic construct
  source 1..50: mol_type = other DNA
attggtaacaatcaattagtctgatatccgttattagtgtgatatttatt

SEQ ID NO: 433 - DNA, 50 bp, synthetic construct
  source 1..50: mol_type = other DNA
aaatccttttccttttttggaatttatcacttatttgttgatgttagtga

SEQ ID NO: 434 - DNA, 50 bp, synthetic construct
  source 1..50: mol_type = other DNA
ttcgaatggtagtcttgttgtttagttttacatttgaattttatatcggt

SEQ ID NO: 435 - DNA, 50 bp, synthetic construct
  source 1..50: mol_type = other DNA
ggattgagcaatcgggttgtgtccgcacgaaattcctttctaattttttt

SEQ ID NO: 436 - DNA, 50 bp, synthetic construct
  source 1..50: mol_type = other DNA
gttgggcctattggtaacagttaatagcttagtatccatgattgggttgt

SEQ ID NO: 437 - DNA, 50 bp, synthetic construct
  source 1..50: mol_type = other DNA
ctaggtatctactttatgacaattatactcattgttcttggttaagagga

SEQ ID NO: 438 - DNA, 50 bp, synthetic construct
  source 1..50: mol_type = other DNA
atgaatgtgtgtgattaaggtgtttggagttatttaaggatatagtgttt

SEQ ID NO: 439 - DNA, 50 bp, synthetic construct
  source 1..50: mol_type = other DNA
gcgcgcagcgccgcaccctttacaccgcatgccttctgggtgaggggccg

SEQ ID NO: 440 - DNA, 50 bp, synthetic construct
  source 1..50: mol_type = other DNA
tcggcggcaccgcgttacttccacttcccggggcgggcccccgggctcca

SEQ ID NO: 441 - DNA, 50 bp, synthetic construct
  source 1..50: mol_type = other DNA
cccccgggcctgtgagccccccggccgccgccgccgctgagacctgcgct

SEQ ID NO: 442 - DNA, 50 bp, synthetic construct
  source 1..50: mol_type = other DNA
ccccgccgcccaggttggggcgcccccaaggcctcgccctccttggtcgc

SEQ ID NO: 443 - DNA, 50 bp, synthetic construct
  source 1..50: mol_type = other DNA
gagagaaggggggaggaagaagggcagccccccgcccagcccccagccgc

SEQ ID NO: 444 - DNA, 50 bp, synthetic construct
  source 1..50: mol_type = other DNA
aaaggcgggcccgggcgggaggggagaaggggcgccccggggcagcaggg

SEQ ID NO: 445 - DNA, 50 bp, synthetic construct
  source 1..50: mol_type = other DNA
acaagggggcggcccaggcggggggcccggcgccaaaaggcgcgccgcgg

SEQ ID NO: 446 - DNA, 50 bp, synthetic construct
  source 1..50: mol_type = other DNA
agggctccacgcaccgcccccgcccgcccgcacagggcgcgcacaccccc

SEQ ID NO: 447 - DNA, 50 bp, synthetic construct
  source 1..50: mol_type = other DNA
agcccagaagggcctcagcccaacgaacccttacgtcgggtttcgcccac

SEQ ID NO: 448 - DNA, 50 bp, synthetic construct
  source 1..50: mol_type = other DNA
ctcgggccgcccacggccgcgcccaggggaggggcgccccggagcgaggt

SEQ ID NO: 449 - DNA, 50 bp, synthetic construct
  source 1..50: mol_type = other DNA
gggccgcgggccggggcaggagcgcagccccagccccgtgcggccggagc

SEQ ID NO: 450 - DNA, 50 bp, synthetic construct
  source 1..50: mol_type = other DNA
gcggttcgcaggcagggcgcgcacgcccgcccgcgctgggcgaggcccct

SEQ ID NO: 451 - DNA, 50 bp, synthetic construct
  source 1..50: mol_type = other DNA
ctttcccccggcgggagagcgggcagtgcaacttgcgtgcaagcacacct

SEQ ID NO: 452 - DNA, 50 bp, synthetic construct
  source 1..50: mol_type = other DNA
gcggcccgcagccctggccccaggccacgcctctcggcaagcagaaccct

SEQ ID NO: 453 - DNA, 50 bp, synthetic construct
  source 1..50: mol_type = other DNA
caggggcaggcaggggcgagccgccccaggggcgcagcaggggcgccgcc

SEQ ID NO: 454 - DNA, 50 bp, synthetic construct
  source 1..50: mol_type = other DNA
ggggccggccgctggccggcggcggcccgcgtgaaggtggcaccgccacg

SEQ ID NO: 455 - DNA, 50 bp, synthetic construct
  source 1..50: mol_type = other DNA
gcgctggcgagagggtggggggaggcagcggagagagccccgggccaccc

SEQ ID NO: 456 - DNA, 50 bp, synthetic construct
  source 1..50: mol_type = other DNA
agggagaggggggcaggcggagggcccgcccgcacccccacccccggccc

SEQ ID NO: 457 - DNA, 50 bp, synthetic construct
  source 1..50: mol_type = other DNA
tcgggaggccggcgcgaaagcggcttagggccccggctccttcggtctat

SEQ ID NO: 458 - DNA, 50 bp, synthetic construct
  source 1..50: mol_type = other DNA
ggcagccagtgcgcagagggctgcttcggctcgcgtgccccagccgccgc

SEQ ID NO: 459 - DNA, 50 bp, synthetic construct
  source 1..50: mol_type = other DNA
gagggaagaaggggcaggcgccccccctgccccgcccacgcccccccgcg

SEQ ID NO: 460 - DNA, 50 bp, synthetic construct
  source 1..50: mol_type = other DNA
ggagcaggagggggaagggggaggcgccccagcccccaagggccccaagc

SEQ ID NO: 461 - DNA, 50 bp, synthetic construct
  source 1..50: mol_type = other DNA
ggggcaggcaggggcaggccgcccaggggcggagcagcgggggggcccac

SEQ ID NO: 462 - DNA, 50 bp, synthetic construct
  source 1..50: mol_type = other DNA
ttaggcgggttcgccttgctatgccgtcgcggcttcctcggagccaaccg

SEQ ID NO: 463 - DNA, 50 bp, synthetic construct
  source 1..50: mol_type = other DNA
gggtggggggcagcggaggcagcagggagagcccccaggcccccgggccc

SEQ ID NO: 464 - DNA, 50 bp, synthetic construct
  source 1..50: mol_type = other DNA
caccccaccgcctttccccccgccagcgcccccggcccggcggggagggt

SEQ ID NO: 465 - DNA, 50 bp, synthetic construct
  source 1..50: mol_type = other DNA
ccggctccctcggcctatgggcagcggcgcgagaggggggccggagaggg

SEQ ID NO: 466 - DNA, 50 bp, synthetic construct
  source 1..50: mol_type = other DNA
acccgcggcctggtggggcggggctcacaatgtcggggggccgtcgtcgc

SEQ ID NO: 467 - DNA, 50 bp, synthetic construct
  source 1..50: mol_type = other DNA
gggaagagccccaccccccctcgcttcggctcgcgtgccccagccgccgc

(19) United States
(12) Patent Application Publication
(10) Pub. No.: US 2025/0011751 A1
(43) Pub. Date: Jan. 9, 2025

(54) PROBES FOR DEPLETING ABUNDANT SMALL NONCODING RNA

(71) Applicant: ILLUMINA, INC., San Diego, CA (US)

(72) Inventors: Terena James, Cambridge (GB); Dunja Vucenovic, Cambridge (GB); Mark Ross, Ware (GB); David McBride, Cambridge (GB); Robert Kuersten, Middleton, WI (US)

(21) Appl. No.: 18/898,412

(22) Filed: Sep. 26, 2024

Related U.S. Application Data
(63) Continuation of application No. PCT/US2023/076101, filed on Oct. 5, 2023.
(60) Provisional application No. 63/378,610, filed on Oct. 6, 2022.

(30) Foreign Application Priority Data
Oct. 5, 2023 (WO) PCT/US2023/076101

(51) Int. Cl. C12N 15/10; C12N 9/22; C12N 15/11

(52) U.S. Cl. CPC C12N 15/10; C12N 9/22; C12N 15/111; C12N 2310/14; C12Y 301/26004

(57) ABSTRACT

Described herein are methods for depleting library fragments prepared from off-target RNA sequences. Libraries enriched or depleted with the present methods may be used for sequencing. Also described are probes and methods for depletion or supplementing depletion of off-target RNA from human and non-human samples.

### CROSS-REFERENCE TO RELATED APPLICATION

This application is a bypass continuation claiming priority to PCT/2023/076101, filed Oct. 5, 2023, which claims the benefit of priority of U.S. Provisional Application No. 63/378,610, filed Oct. 6, 2022, which are incorporated by reference herein in their entireties for any purpose.

### REFERENCE TO ELECTRONIC SEQUENCE LISTING

The application contains a Sequence Listing which has been submitted electronically in .XML format and is hereby incorporated by reference in its entirety. The sequence listing does not go beyond the disclosure of the PCT priority application as filed. Said. XML copy, is named “IP-2342-PCT_ST26.xml” and is 419 kb in size. The sequence listing contained in this .XML file is part of the specification and is hereby incorporated by reference herein in its entirety.

### FIELD

This disclosure relates to methods for depleting library fragments prepared from off-target RNA sequences. Libraries depleted with the present methods may be used to generate sequencing data.

### BACKGROUND

Off-target RNA in a nucleic acid sample, like a nucleic acid sample taken from human cells or tissues, can complicate the analysis of that sample, analysis such as gene expression analysis, microarray analysis, and sequencing of a sample. Off-target RNA, especially if present in abundant amounts, results wasted sequencing reads and highly duplicative results. High levels of duplicates often cause downstream analyses to abort. The amount of off-target RNA contaminating any given sample can be variable. Off-target RNA may comprise abundant small noncoding RNA (sncRNA), as well as other types of RNA species. This is an ever-present problem particularly for tissues that have been fixed, for example fixed by formalin and then embedded in wax such as formalin fixed paraffin embedded (FFPE) tissues from biopsies. Without removing off-target RNA species from FFPE tissues they can interfere with the measurement and characterization of target RNA in the tissue thereby making it extremely difficult to derive medically actionable information from the target RNAs such as disease and cancer identification, potential treatment options and disease or cancer diagnosis and prognosis. While FFPE tissue is an example, the same issues with off-target RNA hold true for samples of all kinds such a blood, cells, and other types of nucleic acid containing samples.

Current commercially available methods for depleting undesired RNA from a nucleic sample include RiboZero® (Epicentre) and NEBNext® rRNA Depletion kits (NEB) and RNA depletion methods as described in U.S. Pat. Nos. 9,745,570 and 9,005,891. However, these methods, while being useful in depleting RNA, have their own disadvantages, including case of use, high sample input requirements, technician hands on time, cost, and/or efficiency in depleting undesired RNA from a sample. What are needed are materials and methods that can more easily or cost effectively deplete off-target RNA species from a sample thereby unlocking information in the target RNA which might have been hidden such as rare or difficult to identify sequence variants. Straightforward and reliable methods as described in this disclosure can greatly increase the availability of target RNA molecules for testing purposes, thereby discovering the information they hold about the sample and the organism from which it derives.

### SUMMARY

In accordance with the description, described herein are methods of depleting abundant small noncoding RNA. These methods may be performed with standard lab equipment, such as flowcells comprised in sequencers. In some embodiments, standard sequencing consumables and platform (i.e., sequencer) can be used as a microfluidic device for depleting library fragments. In some embodiments, depletion is performed after cDNA synthesis and amplification.

Also described are probes that may be used for enzymatic depletion of rRNA from a sample.

Embodiment 1. A method for depleting off-target RNA molecules from a nucleic acid sample comprising providing a probe set comprising at least two DNA probes complementary to discontiguous sequences along the full length of the at least one off-target RNA molecule, wherein the at least one off-target RNA molecule comprises at least one small noncoding RNA chosen from RN7SK, RN7SL1, RN7SL2, RN7SL5P, RPPH1, SNORD3A; (a) contacting a nucleic acid sample comprising at least one target RNA or DNA sequence and at least one off-target RNA molecule with the probe set, thereby hybridizing the DNA probes to the at least one off-target RNA molecule to form DNA:RNA hybrids, wherein each DNA:RNA hybrid is at least 5 bases apart, or at least 10 bases apart, along a given off-target RNA molecule sequence from any other DNA:RNA hybrid; and (b) contacting the DNA:RNA hybrids with a ribonuclease that degrades the RNA from the DNA:RNA hybrids, thereby degrading the off-target RNA molecules in the nucleic acid sample to form a degraded mixture.

Embodiment 2. The method of embodiment 1, wherein at least one small noncoding RNA sequence is chosen from SEQ ID NOS: 1-6.

Embodiment 3. The method of any one of embodiments 1-2, wherein at least one off-target RNA is chosen from the portion of SNORD3A that does not correspond to ALU.

Embodiment 4. The method of any one of embodiments 1-3, wherein the off-target RNA is not MALAT1.

Embodiment 5. The method of any one of embodiments 1-4, wherein the probe length is from 20 to 100 nucleotides.

Embodiment 6. The method of any one of embodiments 1-5, wherein the probe length is from 40 to 60 nucleotides.

Embodiment 7. The method of any one of embodiments 1-6, wherein the probe length is from 40 to 50 nucleotides.

Embodiment 8. The method of any one of embodiments 1-7, wherein at least two probes in the probe set comprise any one of SEQ ID NOs: 8-39.

Embodiment 9. The method of embodiment 8, wherein the probe set comprises five or more, or 10 or more, or 25 or more sequences, or all of the sequences selected from SEQ ID NOs: 8-39.

Embodiment 10. The method of any one of embodiments 1-9, wherein at least two probes in the probe set comprise any one of SEQ ID NOs: 40-467.

Embodiment 11. The method of embodiment 10, wherein the probe set comprises five or more, or 10 or more, or 25 or more sequences, or all of the sequences selected from SEQ ID NOs: 40-467.

Embodiment 12. The method of embodiment 11, wherein the probe set comprises at least 10, at least 50, at least 100, 2 at least 00, at least 300, or at least 400 sequences selected from SEQ ID NOs: 40-467.Embodiment 13. The method of embodiment 11, wherein the probe set comprises: (a) two or more, or five or more, or 10 or more, or 25 or more, or 50 or more, or 100 or more, or 150 or more, or 200 or more, or 250 or more, or 300 or more, or 333 sequences selected from SEQ ID NOs: 40-372; or (b) two or more, or five or more, or 10 or more, or 25 or more, or 50 or more, or 100 or more, or 150 or more, or 200 or more, or 250 or more, or 300 or more, or 350 or more, or 400 or more, or 428 sequences selected from SEQ ID NOs: 40-467; or (c) two or more, or five or more, or 10 or more, or 25 or more, or 50 or more, or 100 or more, or 150 or more, or 200 or more, or 250 or more, or 300 or more, or 350 or more, or 377 sequences selected from SEQ ID NOs: 40-416; or (d) two or more, or five or more, or 10 or more, or 25 or more, or 50 or more, or 100 or more, or 150 or more, or 200 or more, or 250 or more, or 300 or more, or 350 or more, or 384 sequences selected from SEQ ID NOs: 40-372 and SEQ ID NOs: 416-467; or (e) two or more, or five or more, or 10 or more, or 25 or more, or 44 sequences selected from SEQ ID NOs: 41-416; or (f) two or more, or five or more, or 10 or more, or 25 or more, or 50 or more, or 51 sequences selected from SEQ ID NOs: 416-467; (g) or a combination thereof.

Embodiment 14. The method of any one of embodiments 1-13, wherein the nucleic acid sample is an FFPE sample.

Embodiment 15. The method of any one of embodiments 1-13, wherein the probes bind to noncoding RNA molecules leaving a 15 base pair gap between probes.

Embodiment 16. The method of any one of embodiments 1-14, further comprising: c) degrading any remaining DNA probes by contacting the degraded mixture with a DNA digesting enzyme, optionally wherein the DNA digesting enzyme is DNase I, to form a DNA degraded mixture; and d) separating the degraded RNA from the degraded mixture or the DNA degraded mixture.

Embodiment 17. The method of any one of embodiments 1-15, wherein the contacting with the probe set comprises treating the nucleic acid sample with a destabilizer.

Embodiment 18. The method of embodiment 16, wherein with the destabilizer is heat and/or a nucleic acid destabilizing chemical.

Embodiment 19. The method of embodiment 18, wherein the nucleic acid destabilizing chemical is betaine, DMSO, formamide, glycerol, or a derivative thereof, or a mixture thereof.

Embodiment 20. The method of embodiment 19, wherein the nucleic acid destabilizing chemical is formamide, optionally wherein the formamide is present during the contacting with the probe set at a concentration of from about 10 to 45% by volume.

Embodiment 21. The method of embodiment 18, wherein treating the sample with heat comprises applying heat above the melting temperature of the at least one DNA:RNA hybrid.

Embodiment 22. The method of any one of embodiments 1-21, wherein the ribonuclease is RNase H or Hybridase.

Embodiment 23. The method of any one of embodiments 1-22, wherein the nucleic acid sample is from a human.

Embodiment 24. The method of embodiment 23, wherein the nucleic acid sample further comprises nucleic acids of non-human origin.

Embodiment 25. The method of embodiment 24, wherein the nucleic acids of non-human origin are from non-human eukaryotes, bacteria, viruses, plants, soil, or a mixture thereof.

Embodiment 26. The method of any one of embodiments 1-25, wherein the off-target RNA further comprises rRNA, mRNA, tRNA, or a mixture thereof.

Embodiment 27. The method of embodiment 26, wherein the off-target RNA is sncRNA, rRNA, and globin mRNA.

Embodiment 28. The method of embodiment 27, wherein the globin mRNA is hemoglobin mRNA.

Embodiment 29. The method of any one of embodiments 1-28, wherein the probe set further comprises at least two DNA probes that hybridize to at least one off-target RNA molecule selected from 28S, 23S, 18S, 5.8S, 5S, 16S, 12S, HBA-A1, HBA-A2, HBB, HBB-B1, HBB-B2, HBG1, and HBG2.

Embodiment 30. The method of embodiment 29, wherein the probe set further comprises at least two DNA probes that hybridize to two or more off-target RNA molecules selected from 28S, 18S, 5.8S, 5S, 16S, and 12S from humans.

Embodiment 31. The method of any one of embodiments 1-30, wherein the probe set further comprises at least two DNA probes that hybridize to one or more off-target RNA molecules selected from HBA-A1, HBA-A2, HBB, HBG1, and HBG2 from hemoglobin, and 23S, 16S, and 5S from Gram positive or Gram negative bacteria.

Embodiment 32. The method of any one of embodiments 1-31, wherein the probe set further comprises at least two DNA probes that hybridize to one or more off-target RNA molecules from an Archaea species.

Embodiment 33. The method of any one of embodiments 1-32, wherein probes to a particular off-target RNA molecule are complementary to about 65 to 85% of the sequence of the off-target RNA molecule, with gaps of at least 5, or at least 10, or 15 bases between each probe hybridization site.

Embodiment 34. A composition comprising a probe set comprising at least two DNA probes complementary to discontiguous sequences at least 5, or at least 10, or 15 bases apart along the full length of at least one off-target RNA molecule in a nucleic acid sample and a ribonuclease capable of degrading RNA in a DNA:RNA hybrid, wherein the off-target RNA comprises at least one small noncoding RNA chosen from RN7SK, RN7SL1, RN7SL2, RN7SL5P, RPPH1, SNORD3A.

Embodiment 35. The composition of embodiment 34, wherein at least one small noncoding RNA sequence is chosen from SEQ ID NOS: 1-6.

Embodiment 36. The composition of embodiment 34 or 3435 wherein at least one off-target RNA is chosen from the portion of SNORD3A that does not correspond to ALU.

Embodiment 37. The method of any one of embodiments 34-36, wherein the off-target RNA is not MALAT1.

Embodiment 38. The composition of any one of embodiments 34-37, wherein the ribonuclease is RNase H.

Embodiment 39. The composition of any one of embodiments 34-38, wherein each DNA probe is hybridized at least 10 bases apart along the full length of the at least one off-target RNA molecule from any other DNA probe in the probe set.

Embodiment 40. The composition of any one of embodiments 34-39, wherein the composition comprises a destabilizing chemical.

Embodiment 41. The composition of embodiment 40, wherein the destabilizing chemical is formamide.

Embodiment 42. The composition of any one of embodiments 34-41, wherein the off-target RNA further comprises rRNA, mRNA, tRNA, or a mixture thereof.

Embodiment 43. The composition of any one of embodiments 34-41, wherein the off-target RNA is sncRNA, rRNA, and globin mRNA.

Embodiment 44. The composition of any one of embodiments 34-43, wherein the probe set further comprises at least two DNA probes that hybridize to at least one off-target RNA molecule selected from 28S, 23S, 18S, 5.8S, 5S, 16S, 12S, HBA-A1, HBA-A2, HBB, HBG1, and HBG2.

Embodiment 45. The composition of embodiment 44, wherein the probe set further comprises at least two DNA probes that hybridize to two or more off-target RNA molecules selected from 28S, 18S, 5.8S, 5S, 16S, and 12S from humans.

Embodiment 46. The composition of any one of embodiments 34-45, wherein the probe set further comprises at least two DNA probes that hybridize to one or more off-target RNA molecules selected from HBA-A1, HBA-A2, HBB, HBG1, and HBG2 from hemoglobin, and 23S, 16S, and 5S from Gram positive or Gram negative bacteria.

Embodiment 47. The composition of any one of embodiments 34-46, wherein the probe set further comprises at least two DNA probes complementary to one or more rRNA molecules from an Archaea species.

Embodiment 48. The composition of embodiment 47, wherein the probe set further comprises DNA probes that hybridize to one or more off-target RNA molecules from rat and/or mouse, optionally selected from rat 16S, rat 28S, mouse 16S, and mouse 28S, and combinations thereof.

Embodiment 49. The composition of any one of embodiments 34-48, wherein the DNA probes comprise two or more, or five or more, or 10 or more, or 25 or more sequences, or all of the sequences selected from SEQ ID NOs: 8-39.

Embodiment 50. The composition of embodiment 49, wherein the DNA probes further comprise any one of SEQ ID NOs: 40-467.

Embodiment 51. The composition of embodiment 50, wherein the DNA probes further comprise five or more, or 10 or more, or 25 or more sequences, or all of the sequences selected from SEQ ID NOs: 40-467.

Embodiment 52. The composition of embodiment XX, wherein the probe set comprises: (a) two or more, or five or more, or 10 or more, or 25 or more, or 50 or more, or 100 or more, or 150 or more, or 200 or more, or 250 or more, or 300 or more, or 333 sequences selected from SEQ ID NOs: 40-372; or (b) two or more, or five or more, or 10 or more, or 25 or more, or 50 or more, or 100 or more, or 150 or more, or 200 or more, or 250 or more, or 300 or more, or 350 or more, or 400 or more, or 428 sequences selected from SEQ ID NOs: 40-467; or (c) two or more, or five or more, or 10 or more, or 25 or more, or 50 or more, or 100 or more, or 150 or more, or 200 or more, or 250 or more, or 300 or more, or 350 or more, or 377 sequences selected from SEQ ID NOs: 40-416; or (d) two or more, or five or more, or 10 or more, or 25 or more, or 50 or more, or 100 or more, or 150 or more, or 200 or more, or 250 or more, or 300 or more, or 350 or more, or 384 sequences selected from SEQ ID NOs: 40-372 and SEQ ID NOs: 416-467; or (c) two or more, or five or more, or 10 or more, or 25 or more, or 44 sequences selected from SEQ ID NOs: 41-416; or (f) two or more, or five or more, or 10 or more, or 25 or more, or 50 or more, or 51 sequences selected from SEQ ID NOs: 416-467; (g) or a combination thereof.

Embodiment 53. The composition of embodiment 51, wherein the probe set comprises at least 10, at least 50, at least 100, 2 at least 00, at least 300, or at least 400 sequences selected from SEQ ID NOs: 40-467.Embodiment 54. A kit comprising a probe set comprising at least two DNA probes complementary to discontiguous sequences at least 5, or at least 10, or 15 bases apart along the full length of at least one off-target RNA molecule in a nucleic acid sample and a ribonuclease capable of degrading RNA in a DNA:RNA hybrid, wherein the off-target RNA comprises at least one small noncoding RNA chosen from RN7SK, RN7SL1, RN7SL2, RN7SL5P, RPPH1, SNORD3A.

Embodiment 55. The kit of embodiment 54, wherein at least one small noncoding RNA sequence is chosen from SEQ ID NOS: 1-6.

Embodiment 56. The kit of embodiment 54 or 55, wherein at least one off-target RNA is chosen from the portion of SNORD3A that does not correspond to ALU.

Embodiment 57. The kit of any one of embodiments 54-56 wherein the off-target RNA is not MALAT1.

Embodiment 58. The kit of any one of embodiments 54-57, comprising a buffer and nucleic acid purification medium.

Embodiment 59. The kit of any one of embodiments 54-58, further comprising a destabilizing chemical.

Embodiment 60. The kit of any one of embodiments 54-59, wherein the off-target RNA further comprises rRNA, mRNA, tRNA, or a mixture thereof.

Embodiment 61. The kit of any one of embodiments 54-59, wherein the off-target RNA is sncRNA, rRNA and globin mRNA.

Embodiment 62. The kit of any one of embodiments 54-61, wherein the probe set further comprises at least two DNA probes that hybridize to at least one off-target RNA molecule selected from 28S, 23S, 18S, 5.8S, 5S, 16S, 12S, HBA-A1, HBA-A2, HBB, HBG1, and HBG2.

Embodiment 63. The kit of embodiment 62, wherein the probe set further comprises at least two DNA probes that hybridize to two or more off-target RNA molecules selected from 28S, 18S, 5.8S, 5S, 16S, and 12S from humans.

Embodiment 64. The kit of embodiment 62 or 63, wherein the probe set further comprises at least two DNA probes that hybridize to one or more off-target RNA molecules selected from HBA-A1, HBA-A2, HBB, HBG1, and HBG2 from hemoglobin, and 23S, 16S, and 5S from Gram positive or Gram negative bacteria.

Embodiment 65. The kit of any one of embodiments 62-64, wherein the probe set further comprises at least two DNA probes complementary to one or more rRNA molecules from an Archaea species.

Embodiment 66. The kit of embodiment 65, wherein the probe set further comprises DNA probes that hybridize to one or more off-target RNA molecules from rat and/or mouse, optionally selected from rat 16S, rat 28S, mouse 16S, and mouse 28S, and combinations thereof.

Embodiment 67. The kit of any one of embodiments 62-66, wherein the DNA probes comprise two or more, or five or more, or 10 or more, or 25 or more sequences, or all of the sequences selected from SEQ ID NOs: 8-39.

Embodiment 68. The kit of embodiment 67, wherein the DNA probes further comprise any one of SEQ ID NOs: 40-467.

Embodiment 69. The kit of embodiment 68, wherein the DNA probes further comprise five or more, or 10 or more, or 25 or more sequences, or all of the sequences selected from SEQ ID NOs: 40-467.

Embodiment 70. The kit of embodiment 68, wherein the probe set comprises at least 10, at least 50, at least 100, 2 at least 00, at least 300, or at least 400 sequences selected from SEQ ID NOs: 40-467.

The kit of embodiment 68, wherein the probe set comprises: (a) two or more, or five or more, or 10 or more, or 25 or more, or 50 or more, or 100 or more, or 150 or more, or 200 or more, or 250 or more, or 300 or more, or 333 sequences selected from SEQ ID NOs: 40-372; or (b) two or more, or five or more, or 10 or more, or 25 or more, or 50 or more, or 100 or more, or 150 or more, or 200 or more, or 250 or more, or 300 or more, or 350 or more, or 400 or more, or 428 sequences selected from SEQ ID NOs: 40-467; or (c) two or more, or five or more, or 10 or more, or 25 or more, or 50 or more, or 100 or more, or 150 or more, or 200 or more, or 250 or more, or 300 or more, or 350 or more, or 377 sequences selected from SEQ ID NOs: 40-416; or (d) two or more, or five or more, or 10 or more, or 25 or more, or 50 or more, or 100 or more, or 150 or more, or 200 or more, or 250 or more, or 300 or more, or 350 or more, or 384 sequences selected from SEQ ID NOs: 40-372 and SEQ ID NOs: 416-467; or (e) two or more, or five or more, or 10 or more, or 25 or more, or 44 sequences selected from SEQ ID NOs: 41-416; or (f) two or more, or five or more, or 10 or more, or 25 or more, or 50 or more, or 51 sequences selected from SEQ ID NOs: 416-467; (g) or a combination thereof.

Embodiment 72. The kit of embodiment 69 comprising: (a) a probe set comprising SEQ ID NOs: 8-39 and 40-467; (b) a ribonuclease, optionally wherein the ribonuclease is RNase H; (c) a DNase; and (d) RNA purification beads.

Embodiment 73. The kit of embodiment 72, further comprising an RNA depletion buffer, a probe depletion buffer, and a probe removal buffer.

Embodiment 74. A method of supplementing a probe set for use in depleting off-target RNA nucleic acid molecules from a nucleic acid sample comprising: (a) contacting a nucleic acid sample comprising at least one RNA or DNA target sequence and at least one off-target RNA molecule from a first species with a probe set comprising at least two DNA probes complementary to discontiguous sequences along the full length of the at least one off-target RNA molecule from a second species, thereby hybridizing the DNA probes to the off-target RNA molecules to form DNA:RNA hybrids, wherein each DNA:RNA hybrid is at least 5 bases apart, or at least 10 bases apart, along a given off-target RNA molecule sequence from any other DNA:RNA hybrid, wherein the off-target DNA comprises at least one small noncoding RNA chosen from RN7SK, RN7SL1, RN7SL2, RN7SL5P, RPPH1, SNORD3A; (b) contacting the DNA:RNA hybrids with a ribonuclease that degrades the RNA from the DNA:RNA hybrids, thereby degrading the off-target RNA molecules in the nucleic acid sample to form a degraded mixture; (c) separating the degraded RNA from the degraded mixture; (d) sequencing the remaining RNA from the sample; (e) evaluating the remaining RNA sequences for the presence of off-target RNA molecules from the first species, thereby determining gap sequence regions; and (f) supplementing the probe set with additional DNA probes complementary to discontiguous sequences in one or more of the gap sequence regions.

Embodiment 75. The method of embodiment 74, wherein at least one small noncoding RNA sequence is chosen from SEQ ID NOS: 1-6.

Embodiment 76. The method of embodiment 74 or 75 wherein at least one off-target RNA is chosen from the portion of SNORD3A that does not correspond to ALU.

Embodiment 77. The method of any one of embodiments 74-76, wherein the off-target RNA is not MALAT1.

Embodiment 78. The method of any one of embodiments 74-77, wherein the gap sequence regions comprise 50 or more base pairs.

Embodiment 79. The method of any one of embodiments 74-78, wherein the first species is a non-human species and the second species is human.

Embodiment 80. The method of embodiment 79, wherein the first species is rat or mouse.

Embodiment 81. The method of embodiment 79 or embodiment 80, wherein the composition of any one of embodiments 33-51 is used to supply the ribonuclease and the probe set comprising DNA probes complementary to discontiguous sequences along the full length of the at least one off-target RNA molecule of a human.

Embodiment 82. The method of embodiment 80 or embodiment 81, wherein the method is used to identify DNA probes that hybridize to one or more off-target RNA molecules from rat and/or mouse, optionally selected from rat 16S, rat 28S, mouse 16S, and mouse 28S, small noncoding RNA, and combinations thereof.

### BRIEF DESCRIPTION OF THE SEQUENCES

| | SEQ ID | |
|---|---|---|
| Description | NO: | Sequence (3′ to 5′ |
| RN7SK | 1 | GATGTGAGGGCGATCTGGCTGCGACATCTGTCACCCCATTG |
| | | ATCGCCAGGGTTGATTCGGCTGATCTGGCTGGCTAGGCGGG |
| | | TGTCCCCTTCCTCCCTCACCGCTCCATGTGCGTCCCTCCCG |
| | | AAGCTGCGCGCTCGGTCGAAGAGGACGACCATCCCCGATAG |
| | | AGGAGGACCGGTCTTCGGTCAAGGGTATACGAGTAGCTGCG |
| | | CTCCCCTGCTAGAACCTCCAAACAAGCTCTCAAGGTCCATT |
| | | TGTAGGAGAACGTAGGGTAGTCAAGCTTCCAAGACTCCAGA |
| | | CACATCCAAATGAGGCGCTGCATGTGGCAGTCTGCCTTTCT |
| RN7SL1 | 2 | GCCGGGCGCGGTGGCGCGTGCCTGTAGTCCCAGCTACTCGG |
| | | GAGGCTGAGGCTGGAGGATCGCTTGAGTCCAGGAGTTCTGG |
| | | GCTGTAGTGCGCTATGCCGATCGGGTGTCCGCACTAAGTTC |
| | | GGCATCAATATGGTGACCTCCCGGGAGCGGGGGACCACCAG |
| | | GTTGCCTAAGGAGGGGTGAACCGGCCCAGGTCGGAAACGGA |
| | | GCAGGTCAAAACTCCCGTGCTGATCAGTAGTGGGATCGCGC |
| | | CTGTGAATAGCCACTGCACTCCAGCCTGGGCAACATAGCGA |
| | | GACCCCGTCTCT |
| RN7SL2 | 3 | GCCGGGCGCGGTGGCGCGTGCCTGTAGTCCCAGCTACTCGG |
| | | GAGGCTGAGGTGGGAGGATCGCTTGAGCCCAGGAGTTCTGG |
| | | GCTGTAGTGCGCTATGCCGATCGGGTGTCCGCACTAAGTTC |
| | | GGCATCAATATGGTGACCTCCCGGGAGCGGGGGACCACCAG |
| | | GTTGCCTAAGGAGGGGTGAACCGGCCCAGGTCGGAAACGGA |
| | | GCAGGTCAAAACTCCCGTGCTGATCAGTAGTGGGATCGCGC |
| | | CTGTGAATAGCCACTGCACTCCAGCCTGAGCAACATAGCGA |
| | | GACCCCGTCTCTT |
| RN7SL5P | 4 | GCCGGGCGCGGTGGCGCGTGCCTGTGGTCCCAGCTACTCGG |
| | | GAGGCTGAGGCTGGAGGATCGCTTGAGTCCAGGAGTTCTGG |
| | | GCTGTAGTGCGCTATGCCGATCGGGTGTCCGCACTAAGTTC |
| | | GGCATCAATATGGTGACCTCCCGGGAGCGGGGGACCACCAG |
| | | GTTGCCTAAGGAGGGGTGAACCGGCCCAGGTCGGAAACGGA |
| | | GCAGGTCAAAACTCCCGTGCTGATCAGTAGAAGTCTGTAAT |
| | | GCTACTGGTGTCCCCTAATTTTCTTATAGCCACAGTTCCTT |
| | | TCGCCTGAGCTCATTACAGAGACAAATATCCATT |
| RPPH1 | 5 | GGCGGAGGGAAGCTCATCAGTGGGGCCACGAGCTGAGTGCG |
| | | TCCTGTCACTCCACTCCCATGTCCCTTGGGAAGGTCTGAGA |
| | | CTAGGGCCAGAGGCGGCCCTAACAGGGCTCTCCCTGAGCTT |
| | | CGGGGAGGTGAGTTCCCAGAGAACGGGGCTCCGCGCGAGGT |
| | | CAGACTGGGCAGGAGATGCCGTGGACCCCGCCCTTCGGGGA |
| | | GGGGCCCGGCGGATGCCTCCTTTGCCGGAGCTTGGAACAGA |
| | | CTCACGGCCAGCGAAGTGAGTTCAATGGCTGAGGTGAGGTA |
| | | CCCCGCAGGGGACCTCATAACCCAATTCAGACTACTCTCCT |
| | | CCGCC |
| SNORD3A | 6 | AAGACTATACTTTCAGGGATCATTTCTATAGTGTGTTACTA |
| with the ALU | | GAGAAGTTTCTCTGAACGTGTAGAGCACCGAAAACCACGAG |
| region in bold | | GAAGAGAGGTAGCGTTTTCTCCTGAGCGTGAAGCCGGCTTT |
| and italics, in | | CTGGCGTTGCTTGGCTGCAACTGCCGTCAGCCATTGATGAT |
| some | | CGTTCTTCTCTCCGTATTGGGGAGTGAGAGGGAGAGAACGC |
| embodiments | | GGTCTGAGTGGTTTTTCCTTCTTGATGGCTCAATGACAGAG |
| the ALU region | | ACTAGCTCGTAAACTCCGGGGCGTTTCTGGGCTGTTCGCTC |
| was not used to | | CTGCTTGGCATGTCGCGAGAAAGGTTTTCGCCTCCTGTTTC |
| generate probes | | AGCGGTGACGGCTCTTGGGTTTTCTCGGGGTGGCTTTTTAA |
| because it is a | | TTTTAGTCTTGGCGCGAGGCGGGGGATGCTGTGTGGCACCT |
| repetitive | | CCTATTGTCTCTTTTTGCGTTTTCTCCCATTCTCGCTCCCT |
| region in other | | CTTTTGTCGCCGTTTCCCGCCCGCCACTCCCACCCCCAGAC |
| areas of the | | GGGGTCTCCGGGTCTCTTGTTCTGTCTGCCGGCCCCGGCTG |
| genome. | | GATTGCAGTGGCGCGATCTCGGCTCCTAGCAACATCTGCCT |
| | | CCCGGGCTCAAGCGAGTCTCCCGCCTAAGCCCTCCCGAGTA |
| | | GCCGGGGCTTAAAGGCGCACACGCCACTCCAGGCTTTTTTT |
| | | TTTTTTTTTTTTTTTTTTTTGGCAGAAACGGGGTGTCAGCA |
| | | TG |
| Reverse | 7 | AGAAAGGCAGACTGCCACATGCAGCGCCTCATTTGGATGTG |
| complement of | | TCTGGAGTCTTGGAAGCTTGACTACCCTACGTTCTCCTACA |
| RN7SK with | | AATGGACCTTGAGAGCTTGTTTGGAGGTTCTAGCAGGGGAG |
| probe | | CGCAGCTACTCGTATACCCTTGACCGAAGACCGGTCCTCCT |
| sequences in | | CTATCGGGGATGGTCGTCCTCTTCGACCGAGCGCGCAGCTT |
| bold and italics | | CGGGAGGGACGCACATGGAGCGGTGAGGGAGGAAGGGGACA |
| (and with gaps | | CCCGCCTAGCCAGCCAGATCAGCCGAATCAACCCTGGCGAT |
| between the | | CAATGGGGTGACAGATGTCGCAGCCAGATCGCCCTCACATC |
| probes) | | |
| Probe for | 8 | AGAAAGGCAGACTGCCACATGCAGCGCCTCATTTGGATGTG |
| RN7SK | | TCTGGAGTC |
| Probe for | 9 | CCCTACGTTCTCCTACAAATGGACCTTGAGAGCTTGTTTGG |
| RN7SK | | AGGTTCTAG |
| Probe for | 10 | ACTCGTATACCCTTGACCGAAGACCGGTCCTCCTCTATCGG |
| RN7SK | | GGATGGTCG |
| Probe for | 11 | CGCGCAGCTTCGGGAGGGACGCACATGGAGCGGTGAGGGAG |
| RN7SK | | GAAGGGGAC |
| Probe for | 12 | CAGATCAGCCGAATCAACCCTGGCGATCAATGGGGTGACAG |
| RN7SK | | ATGTCGCAG |
| Probe | 13 | AGAGACGGGGTCTCGCTATGTTGCCCAGGCTGGAGTGCAGT |
| for RN7SL1 | | GGCTATTCA |
| Probe for | 14 | TACTGATCAGCACGGGAGTTTTGACCTGCTCCGTTTCCGAC |
| RN7SL1 | | CTGGGCCGG |
| Probe for | 15 | GCAACCTGGTGGTCCCCCGCTCCCGGGAGGTCACCATATTG |
| RN7SL1 | | ATGCCGAAC |
| Probe for | 16 | GATCGGCATAGCGCACTACAGCCCAGAACTCCTGGACTCAA |
| RN7SL1 | | GCGATCCTC |
| Probe for | 17 | AAGAGACGGGGTCTCGCTATGTTGCTCAGGCTGGAGTGCAG |
| RN7SL2 | | TGGCTATTC |
| Probe for | 18 | CTACTGATCAGCACGGGAGTTTTGACCTGCTCCGTTTCCGA |
| RN7SL2 | | CCTGGGCCG |
| Probe | 19 | GGCAACCTGGTGGTCCCCCGCTCCCGGGAGGTCACCATATT |
| for RN7SL2 | | GATGCCGAA |
| Probe | 20 | CGATCGGCATAGCGCACTACAGCCCAGAACTCCTGGGCTCA |
| for RN7SL2 | | AGCGATCCT |
| Probe | 21 | AATGGATATTTGTCTCTGTAATGAGCTCAGGCGAAAGGAAC |
| for RN7SL5P | | TGTGGCTAT |
| Probe | 22 | CACCAGTAGCATTACAGACTTCTACTGATCAGCACGGGAGT |
| for RN7SL5P | | TTTGACCTG |
| Probe | 23 | GGGCCGGTTCACCCCTCCTTAGGCAACCTGGTGGTCCCCCG |
| for RN7SL5P | | CTCCCGGGA |
| Probe | 24 | GCCGAACTTAGTGCGGACACCCGATCGGCATAGCGCACTAC |
| for RN7SL5P | | AGCCCAGAA |
| Probe | 25 | GATCCTCCAGCCTCAGCCTCCCGAGTAGCTGGGACCACAGG |
| for RN7SL5P | | CACGCGCCA |
| Probe | 26 | GGCGGAGGAGAGTAGTCTGAATTGGGTTATGAGGTCCCCTG |
| for RPPH1 | | CGGGGTACC |
| Probe | 27 | AACTCACTTCGCTGGCCGTGAGTCTGTTCCAAGCTCCGGCA |
| for RPPH1 | | AAGGAGGCA |
| Probe | 28 | CCCGAAGGGCGGGGTCCACGGCATCTCCTGCCCAGTCTGAC |
| for RPPH1 | | CTCGCGCGG |
| Probe | 29 | GAACTCACCTCCCCGAAGCTCAGGGAGAGCCCTGTTAGGGC |
| for RPPH1 | | CGCCTCTGG |
| Probe | 30 | TTCCCAAGGGACATGGGAGTGGAGTGACAGGACGCACTCAG |
| for RPPH1 | | CTCGTGGCC |
| Probe | 31 | CCCGGAGACCCCGTCTGGGGGTGGGAGTGGCGGGGGGAAA |
| for SNORD3A | | CGGCGACAA |
| Probe | 32 | TGGGAGAAAACGCAAAAAGAGACAATAGGAGGTGCCACACA |
| for SNORD3A | | GCATCCCCC |
| Probe | 33 | TAAAATTAAAAAGCCACCCCGAGAAAACCCAAGAGCCGTCA |
| for SNORD3A | | CCGCTGAAA |
| Probe | 34 | TTTCTCGCGACATGCCAAGCAGGAGCGAACAGCCCAGAAAC |
| for SNORD3A | | GCCCCGGAG |
| Probe | 35 | CTGTCATTGAGCCATCAAGAAGGAAAAACCACTCAGACCGC |
| for SNORD3A | | GTTCTCTCC |
| Probe for | 36 | ACGGAGAGAAGAACGATCATCAATGGCTGACGGCAGTTGCA |
| SNORD3A | | GCCAAGCAA |
| Probe for | 37 | TTCACGCTCAGGAGAAAACGCTACCTCTCTTCCTCGTGGTT |
| SNORD3A | | TTCGGTGCT |
| Probe for | 38 | AAACTTCTCTAGTAACACACTATAGAAATGATCCCTGAAAG |
| SNORD3A | | TATAGTCTT |
| (additional | | |
| probe added at | | |
| start of | | |
| SNORD3A | | |
| transcript) | | |
| Probe for | 39 | CTCAGCCTCCCGAGTAGCTGGGACTACAGGCACGCGCCACC |
| RN7SL1 and | | GCGCCCGGC |
| RN7SL2 | | |
| (additional | | |
| probe added at | | |
| start of | | |
| RN7SL1 and | | |
| RN7SL2 | | |
| transcript) | | |

Additional Probes

| | | |
|---|---|---|
| 12S_P1 | 40 | GTTCGTCCAAGTGCACTTTCCAGTACACTTACCATGTTACG |
| | | ACTTGTCTC |
| 12S_P2 | 41 | TAGGGGTTTTAGTTAAATGTCCTTTGAAGTATACTTGAGGA |
| | | GGGTGACGG |
| 12S_P3 | 42 | TTCAGGGCCCTGTTCAACTAAGCACTCTACTCTCAGTTTAC |
| | | TGCTAAATC |
| 12S_P4 | 43 | AGTTTCATAAGGGCTATCGTAGTTTTCTGGGGTAGAAAATG |
| | | TAGCCCATT |
| 12S_P5 | 44 | GGCTACACCTTGACCTAACGTCTTTACGTGGGTACTTGCGC |
| | | TTACTTTGT |
| 12S_P6 | 45 | TTGCTGAAGATGGCGGTATATAGGCTGAGCAAGAGGTGGTG |
| | | AGGTTGATC |
| 12S_P7 | 46 | CAGAACAGGCTCCTCTAGAGGGATATGAAGCACCGCCAGGT |
| | | CCTTTGAGT |
| 12S_P8 | 47 | GTAGTGTTCTGGCGAGCAGTTTTGTTGATTTAACTGTTGAG |
| | | GTTTAGGGC |
| 12S_P9 | 48 | ATCTAATCCCAGTTTGGGTCTTAGCTATTGTGTGTTCAGAT |
| | | ATGTTAAAG |
| 12S_P10 | 49 | ATTTTGTGTCAACTGGAGTTTTTTACAACTCAGGTGAGTTT |
| | | TAGCTTTAT |
| 12S_P11 | 50 | CTAAAACACTCTTTACGCCGGCTTCTATTGACTTGGGTTAA |
| | | TCGTGTGAC |
| 12S_P12 | 51 | GAAATTGACCAACCCTGGGGTTAGTATAGCTTAGTTAAACT |
| | | TTCGTTTAT |
| 12S_P13 | 52 | ACTGCTGTTTCCCGTGGGGGTGTGGCTAGGCTAAGCGTTTT |
| | | GAGCTGCAT |
| 12S_P14 | 53 | GCTTGTCCCTTTTGATCGTGGTGATTTAGAGGGTGAACTCA |
| | | CTGGAACGG |
| 12S_P15 | 54 | TAATCTTACTAAGAGCTAATAGAAAGGCTAGGACCAAACCT |
| | | ATTTGTTTA |
| 16S_P1 | 55 | AAACCCTGTTCTTGGGTGGGTGTGGGTATAATACTAAGTTG |
| | | AGATGATAT |
| 16S_P2 | 56 | GCGCTTTGTGAAGTAGGCCTTATTTCTCTTGTCCTTTCGTA |
| | | CAGGGAGGA |
| 16S_P3 | 57 | AAACCGACCTGGATTACTCCGGTCTGAACTCAGATCACGTA |
| | | GGACTTTAA |
| 16S_P4 | 58 | ACCTTTAATAGCGGCTGCACCATCGGGATGTCCTGATCCAA |
| | | CATCGAGGT |
| 16S_P5 | 59 | TGATATGGACTCTAGAATAGGATTGCGCTGTTATCCCTAGG |
| | | GTAACTTGT |
| 16S_P6 | 60 | ATTGGATCAATTGAGTATAGTAGTTCGCTTTGACTGGTGAA |
| | | GTCTTAGCA |
| 16S_P7 | 61 | TTGGGTTCTGCTCCGAGGTCGCCCCAACCGAAATTTTTAAT |
| | | GCAGGTTTG |
| 16S_P8 | 62 | TGGGTTTGTTAGGTACTGTTTGCATTAATAAATTAAAGCTC |
| | | CATAGGGTC |
| 16S_P9 | 63 | GTCATGCCCGCCTCTTCACGGGCAGGTCAATTTCACTGGTT |
| | | AAAAGTAAG |
| 16S_P10 | 64 | CGTGGAGCCATTCATACAGGTCCCTATTTAAGGAACAAGTG |
| | | ATTATGCTA |
| 16S_P11 | 65 | GGTACCGCGGCCGTTAAACATGTGTCACTGGGCAGGCGGTG |
| | | CCTCTAATA |
| 16S_P12 | 66 | GTGATGTTTTTGGTAAACAGGCGGGGTAAGGTTTGCCGAGT |
| | | TCCTTTTAC |
| 16S_P13 | 67 | CTTATGAGCATGCCTGTGTTGGGTTGACAGTGAGGGTAATA |
| | | ATGACTTGT |
| 16S_P14 | 68 | ATTGGGCTGTTAATTGTCAGTTCAGTGTTTTGATCTGACGC |
| | | AGGCTTATG |
| 16S_P15 | 69 | TCATGTTACTTATACTAACATTAGTTCTTCTATAGGGTGAT |
| | | AGATTGGTC |
| 16S_P16 | 70 | AGTTCAGTTATATGTTTGGGATTTTTTAGGTAGTGGGTGTT |
| | | GAGCTTGAA |
| 16S_P17 | 71 | TGGCTGCTTTTAGGCCTACTATGGGTGTTAAATTTTTTACT |
| | | CTCTCTACA |
| 16S_P18 | 72 | GTCCAAAGAGCTGTTCCTCTTTGGACTAACAGTTAAATTTA |
| | | CAAGGGGAT |
| 16S_P19 | 73 | GGCAAATTTAAAGTTGAACTAAGATTCTATCTTGGACAACC |
| | | AGCTATCAC |
| 16S_P20 | 74 | TGTCGCCTCTACCTATAAATCTTCCCACTATTTTGCTACAT |
| | | AGACGGGTG |
| 16S_P21 | 75 | TCTTAGGTAGCTCGTCTGGTTTCGGGGGTCTTAGCTTTGGC |
| | | TCTCCTTGC |
| 16S_P22 | 76 | TAATTCATTATGCAGAAGGTATAGGGGTTAGTCCTTGCTAT |
| | | ATTATGCTT |
| 16S_P23 | 77 | TCTTTCCCTTGCGGTACTATATCTATTGCGCCAGGTTTCAA |
| | | TTTCTATCG |
| 16S_P24 | 78 | GGTAAATGGTTTGGCTAAGGTTGTCTGGTAGTAAGGTGGAG |
| | | TGGGTTTGG |
| 18S_P1 | 79 | TAATGATCCTTCCGCAGGTTCACCTACGGAAACCTTGTTAC |
| | | GACTTTTAC |
| 18S_P2 | 80 | AAGTTCGACCGTCTTCTCAGCGCTCCGCCAGGGCCGTGGGC |
| | | CGACCCCGG |
| 18S_P3 | 81 | GGCCTCACTAAACCATCCAATCGGTAGTAGCGACGGGCGGT |
| | | GTGTACAAA |
| 18S_P4 | 82 | CAACGCAAGCTTATGACCCGCACTTACTCGGGAATTCCCTC |
| | | GTTCATGGG |
| 18S_P5 | 83 | CCGATCCCCATCACGAATGGGGTTCAACGGGTTACCCGCGC |
| | | CTGCCGGCG |
| 18S_P6 | 84 | CTGAGCCAGTCAGTGTAGCGCGCGTGCAGCCCCGGACATCT |
| | | AAGGGCATC |
| 18S_P7 | 85 | CTCAATCTCGGGTGGCTGAACGCCACTTGTCCCTCTAAGAA |
| | | GTTGGGGGA |
| 18S_P8 | 86 | GGTCGCGTAACTAGTTAGCATGCCAGAGTCTCGTTCGTTAT |
| | | CGGAATTAA |
| 18S_P9 | 87 | CACCAACTAAGAACGGCCATGCACCACCACCCACGGAATCG |
| | | AGAAAGAGC |
| 18S_P10 | 88 | CCTGTCCGTGTCCGGGCCGGGTGAGGTTTCCCGTGTTGAGT |
| | | CAAATTAAG |
| 18S_P11 | 89 | CTGGTGGTGCCCTTCCGTCAATTCCTTTAAGTTTCAGCTTT |
| | | GCAACCATA |
| 18S_P12 | 90 | AAAGACTTTGGTTTCCCGGAAGCTGCCCGGCGGGTCATGGG |
| | | AATAACGCC |
| 18S_P13 | 91 | GGCATCGTTTATGGTCGGAACTACGACGGTATCTGATCGTC |
| | | TTCGAACCT |
| 18S_P14 | 92 | GATTAATGAAAACATTCTTGGCAAATGCTTTCGCTCTGGTC |
| | | CGTCTTGCG |
| 18S_P15 | 93 | CACCTCTAGCGGCGCAATACGAATGCCCCCGGCCGTCCCTC |
| | | TTAATCATG |
| 18S_P16 | 94 | ACCAACAAAATAGAACCGCGGTCCTATTCCATTATTCCTAG |
| | | CTGCGGTAT |
| 18S_P17 | 95 | CTGCTTTGAACACTCTAATTTTTTCAAAGTAAACGCTTCGG |
| | | GCCCCGCGG |
| 18S_P18 | 96 | GCATCGAGGGGGCGCCGAGAGGCAAGGGGCGGGGACGGGCG |
| | | GTGGCTCGC |
| 18S_P19 | 97 | CCGCCCGCTCCCAAGATCCAACTACGAGCTTTTTAACTGCA |
| | | GCAACTTTA |
| 18S_P20 | 98 | GCTGGAATTACCGCGGCTGCTGGCACCAGACTTGCCCTCCA |
| | | ATGGATCCT |
| 18S_P21 | 99 | AGTGGACTCATTCCAATTACAGGGCCTCGAAAGAGTCCTGT |
| | | ATTGTTATT |
| 18S_P22 | 100 | CCCGGGTCGGGAGTGGGTAATTTGCGCGCCTGCTGCCTTCC |
| | | TTGGATGTG |
| 18S_P23 | 101 | GCTCCCTCTCCGGAATCGAACCCTGATTCCCCGTCACCCGT |
| | | GGTCACCAT |
| 18S_P24 | 102 | TACCATCGAAAGTTGATAGGGCAGACGTTCGAATGGGTCGT |
| | | CGCCGCCAC |
| 18S_P25 | 103 | GGCCCGAGGTTATCTAGAGTCACCAAAGCCGCCGGCGCCCG |
| | | CCCCCCGGC |
| 18S_P26 | 104 | GCTGACCGGGTTGGTTTTGATCTGATAAATGCACGCATCCC |
| | | CCCCGCGAA |
| 18S_P27 | 105 | TCGGCATGTATTAGCTCTAGAATTACCACAGTTATCCAAGT |
| | | AGGAGAGGA |
| 18S_P28 | 106 | AACCATAACTGATTTAATGAGCCATTCGCAGTTTCACTGTA |
| | | CCGGCCGTG |
| 18S_P29 | 107 | ATGGCTTAATCTTTGAGACAAGCATATGCTACTGGCAGGAT |
| | | CAACCAGGT |
| 28S_P1 | 108 | GACAAACCCTTGTGTCGAGGGCTGACTTTCAATAGATCGCA |
| | | GCGAGGGAG |
| 28S_P2 | 109 | CGAAACCCCGACCCAGAAGCAGGTCGTCTACGAATGGTTTA |
| | | GCGCCAGGT |
| 28S_P3 | 110 | GGTGCGTGACGGGCGAGGGGGCGGCCGCCTTTCCGGCCGCG |
| | | CCCCGTTTC |
| 28S_P4 | 111 | CTCCGCACCGGACCCCGGTCCCGGCGCGCGGCGGGGCACGC |
| | | GCCCTCCCG |
| 28S_P5 | 112 | AGGGGGGGGCGGCCCGCCGGCGGGGACAGGCGGGGGACCGG |
| | | CTATCCGAG |
| 28S_P6 | 113 | GCGGCGCTGCCGTATCGTTCGCCTGGGCGGGATTCTGACTT |
| | | AGAGGCGTT |
| 28S_P7 | 114 | AGATGGTAGCTTCGCCCCATTGGCTCCTCAGCCAAGCACAT |
| | | ACACCAAAT |
| 28S_P8 | 115 | TCCTCTCGTACTGAGCAGGATTACCATGGCAACAACACATC |
| | | ATCAGTAGG |
| 28S_P9 | 116 | CTCACGACGGTCTAAACCCAGCTCACGTTCCCTATTAGTGG |
| | | GTGAACAAT |
| 28S_P10 | 117 | TTCTGCTTCACAATGATAGGAAGAGCCGACATCGAAGGATC |
| | | AAAAAGCGA |
| 28S_P11 | 118 | TTGGCCGCCACAAGCCAGTTATCCCTGTGGTAACTTTTCTG |
| | | ACACCTCCT |
| 28S_P12 | 119 | GGTCAGAAGGATCGTGAGGCCCCGCTTTCACGGTCTGTATT |
| | | CGTACTGAA |
| 28S_P13 | 120 | AGCTTTTGCCCTTCTGCTCCACGGGAGGTTTCTGTCCTCCC |
| | | TGAGCTCGC |
| 28S_P14 | 121 | TTACCGTTTGACAGGTGTACCGCCCCAGTCAAACTCCCCAC |
| | | CTGGCACTG |
| 28S_P15 | 122 | GCGCCCGGCCGGGCGGGCGCTTGGCGCCAGAAGCGAGAGCC |
| | | CCTCGGGCT |
| 28S_P16 | 123 | CCGGGTCAGTGAAAAAACGATCAGAGTAGTGGTATTTCACC |
| | | GGCGGCCCG |
| 28S_P17 | 124 | CGCCCCGGGCCCCTCGCGGGGACACCGGGGGGGCGCCGGGG |
| | | GCCTCCCAC |
| 28S_P18 | 125 | CATGTCTCTTCACCGTGCCAGACTAGAGTCAAGCTCAACAG |
| | | GGTCTTCTT |
| 28S_P19 | 126 | CCAAGCCCGTTCCCTTGGCTGTGGTTTCGCTGGATAGTAGG |
| | | TAGGGACAG |
| 28S_P20 | 127 | TCCATTCATGCGCGTCACTAATTAGATGACGAGGCATTTGG |
| | | CTACCTTAA |
| 28S_P21 | 128 | TCCCGCCGTTTACCCGCGCTTCATTGAATTTCTTCACTTTG |
| | | ACATTCAGA |
| 28S_P22 | 129 | CACATCGCGTCAACACCCGCCGCGGGCCTTCGCGATGCTTT |
| | | GTTTTAATT |
| 28S_P23 | 130 | CCTGGTCCGCACCAGTTCTAAGTCGGCTGCTAGGCGCCGGC |
| | | CGAGGCGAG |
| 28S_P24 | 131 | CGGCCCCGGGGGCGGACCCGGCGGGGGGGACCGGCCCGCGG |
| | | CCCCTCCGC |
| 28S_P25 | 132 | CCGCCGCGCGCCGAGGAGGAGGGGGGAACGGGGGGCGGACG |
| | | GGGCCGGGG |
| 28S_P26 | 133 | ACGAACCGCCCCGCCCCGCCGCCCGCCGACCGCCGCCGCCC |
| | | GACCGCTCC |
| 28S_P27 | 134 | CGCGCGCGACCGAGACGTGGGGTGGGGGTGGGGGGCGCGCC |
| | | GCGCCGCCG |
| 28S_P28 | 135 | GCGGCCGCGACGCCCGCCGCAGCTGGGGCGATCCACGGGAA |
| | | GGGCCCGGC |
| 28S_P29 | 136 | GCGCCGCCGCCGGCCCCCCGGGTCCCCGGGGCCCCCCTCGC |
| | | GGGGACCTG |
| 28S_P30 | 137 | CCGGCGGCCGCCGCGCGGCCCCTGCCGCCCCGACCCTTCTC |
| | | CCCCCGCCG |
| 28S_P31 | 138 | CTCCCCCGGGGAGGGGGGAGGACGGGGAGCGGGGGAGAGAG |
| | | AGAGAGAGA |
| 28S_P32 | 139 | AGGGAGCGAGCGGCGCGCGCGGGTGGGGCGGGGGAGGGCCG |
| | | CGAGGGGGG |
| 28S_P33 | 140 | GGGGGCGCGCGCCTCGTCCAGCCGCGGCGCGCGCCCAGCCC |
| | | CGCTTCGCG |
| 28S_P34 | 141 | CCCAGCCCTTAGAGCCAATCCTTATCCCGAAGTTACGGATC |
| | | CGGCTTGCC |
| 28S_P35 | 142 | CATTGTTCCAACATGCCAGAGGCTGTTCACCTTGGAGACCT |
| | | GCTGCGGAT |
| 28S_P36 | 143 | CGCGAGATTTACACCCTCTCCCCCGGATTTTCAAGGGCCAG |
| | | CGAGAGCTC |
| 28S_P37 | 144 | AACCGCGACGCTTTCCAAGGCACGGGCCCCTCTCTCGGGGC |
| | | GAACCCATT |
| 28S_P38 | 145 | CTTCACAAAGAAAAGAGAACTCTCCCCGGGGCTCCCGCCGG |
| | | CTTCTCCGG |
| 28S_P39 | 146 | CGCACTGGACGCCTCGCGGCGCCCATCTCCGCCACTCCGGA |
| | | TTCGGGGAT |
| 28S_P40 | 147 | TTTCGATCGGCCGAGGGCAACGGAGGCCATCGCCCGTCCCT |
| | | TCGGAACGG |
| 28S_P41 | 148 | CAGGACCGACTGACCCATGTTCAACTGCTGTTCACATGGAA |
| | | CCCTTCTCC |
| 28S_P42 | 149 | GTTCTCGTTTGAATATTTGCTACTACCACCAAGATCTGCAC |
| | | CTGCGGCGG |
| 28S_P43 | 150 | CGCCCTAGGCTTCAAGGCTCACCGCAGCGGCCCTCCTACTC |
| | | GTCGCGGCG |
| 28S_P44 | 151 | TCCGGGGGCGGGGAGCGGGGCGTGGGCGGGAGGAGGGGAGG |
| | | AGGCGTGGG |
| 28S_P45 | 152 | AGGACCCCACACCCCCGCCGCCGCCGCCGCCGCCGCCCTCC |
| | | GACGCACAC |
| 28S_P46 | 153 | GCGCGCCGCCCCCGCCGCTCCCGTCCACTCTCGACTGCCGG |
| | | CGACGGCCG |
| 28S_P47 | 154 | CTCCAGCGCCATCCATTTTCAGGGCTAGTTGATTCGGCAGG |
| | | TGAGTTGTT |
| 28S_P48 | 155 | GATTCCGACTTCCATGGCCACCGTCCTGCTGTCTATATCAA |
| | | CCAACACCT |
| 28S_P49 | 156 | GAGCGTCGGCATCGGGCGCCTTAACCCGGCGTTCGGTTCAT |
| | | CCCGCAGCG |
| 28S_P50 | 157 | AAAAGTGGCCCACTAGGCACTCGCATTCCACGCCCGGCTCC |
| | | ACGCCAGCG |
| 28S_P51 | 158 | CCATTTAAAGTTTGAGAATAGGTTGAGATCGTTTCGGCCCC |
| | | AAGACCTCT |
| 28S_P52 | 159 | CGGATAAAACTGCGTGGCGGGGGTGCGTCGGGTCTGCGAGA |
| | | GCGCCAGCT |
| 28S_P53 | 160 | TCGGAGGGAACCAGCTACTAGATGGTTCGATTAGTCTTTCG |
| | | CCCCTATAC |
| 28S_P54 | 161 | GATTTGCACGTCAGGACCGCTACGGACCTCCACCAGAGTTT |
| | | CCTCTGGCT |
| 28S_P55 | 162 | ATAGTTCACCATCTTTCGGGTCCTAACACGTGCGCTCGTGC |
| | | TCCACCTCC |
| 28S_P56 | 163 | AGACGGGCCGGTGGTGCGCCCTCGGCGGACTGGAGAGGCCT |
| | | CGGGATCCC |
| 28S_P57 | 164 | CGCGCCGGCCTTCACCTTCATTGCGCCACGGCGGCTTTCGT |
| | | GCGAGCCCC |
| 28S_P58 | 165 | TTAGACTCCTTGGTCCGTGTTTCAAGACGGGTCGGGTGGGT |
| | | AGCCGACGT |
| 28S_P59 | 166 | GCGCTCGCTCCGCCGTCCCCCTCTTCGGGGGACGCGCGCGT |
| | | GGCCCCGAG |
| 28S_P60 | 167 | CCCGACGGCGCGACCCGCCCGGGGCGCACTGGGGACAGTCC |
| | | GCCCCGCCC |
| 28S_P61 | 168 | GCACCCCCCCCGTCGCCGGGGCGGGGGCGCGGGGAGGAGGG |
| | | GTGGGAGAG |
| 28S_P62 | 169 | AGGGGTGGCCCGGCCCCCCCACGAGGAGACGCCGGCGCGCC |
| | | CCCGCGGGG |
| 28S_P63 | 170 | GGGGATTCCCCGCGGGGGTGGGCGCCGGGAGGGGGGAGAGC |
| | | GCGGCGACG |
| 28S_P64 | 171 | GCCCCGGGATTCGGCGAGTGCTGCTGCCGGGGGGGCTGTAA |
| | | CACTCGGGG |
| 28S_P65 | 172 | CCGCCCCCGCCGCCGCCGCCACCGCCGCCGCCGCCGCCGCC |
| | | CCGACCCGC |
| 28S_P66 | 173 | AGGACGCGGGGCCGGGGGGCGGAGACGGGGGAGGAGGAGGA |
| | | CGGACGGAC |
| 28S_P67 | 174 | AGCCACCTTCCCCGCCGGGCCTTCCCAGCCGTCCCGGAGCC |
| | | GGTCGCGGC |
| 28S_P68 | 175 | AAATGCGCCCGGCGGCGGCCGGTCGCCGGTCGGGGGACGGT |
| | | CCCCCGCCG |
| 28S_P69 | 176 | CCGCCCGCCCACCCCCGCACCCGCCGGAGCCCGCCCCCTCC |
| | | GGGGAGGAG |
| 28S_P70 | 177 | GGGAAGGGAGGGCGGGTGGAGGGGTCGGGAGGAACGGGGGG |
| | | CGGGAAAGA |
| 28S_P71 | 178 | ACACGGCCGGACCCGCCGCCGGGTTGAATCCTCCGGGCGGA |
| | | CTGCGCGGA |
| 28S_P72 | 179 | TCTTAACGGTTTCACGCCCTCTTGAACTCTCTCTTCAAAGT |
| | | TCTTTTCAA |
| 28S_P73 | 180 | CTTGTTGACTATCGGTCTCGTGCCGGTATTTAGCCTTAGAT |
| | | GGAGTTTAC |
| 28S_P74 | 181 | GCATTCCCAAGCAACCCGACTCCGGGAAGACCCGGGCGCGC |
| | | GCCGGCCGC |
| 28S_P75 | 182 | GTCCACGGGCTGGGCCTCGATCAGAAGGACTTGGGCCCCCC |
| | | ACGAGCGGC |
| 28S_P76 | 183 | TTCCGTACGCCACATGTCCCGCGCCCCGCGGGGCGGGGATT |
| | | CGGCGCTGG |
| 28S_P77 | 184 | CTCGCCGTTACTGAGGGAATCCTGGTTAGTTTCTTTTCCTC |
| | | CGCTGACTA |
| 28S_P78 | 185 | GCGGGTCGCCACGTCTGATCTGAGGTCGCGTCTCGGAGGGG |
| | | GACGGGCCG |
| 5.8S_P1 | 186 | AAGCGACGCTCAGACAGGCGTAGCCCCGGGAGGAACCCGGG |
| | | GCCGCAAGT |
| 5.8S_P3 | 187 | GCAGCTAGCTGCGTTCTTCATCGACGCACGAGCCGAGTGAT |
| | | CCACCGCTA |
| 5S_P1 | 188 | AAAGCCTACAGCACCCGGTATTCCCAGGCGGTCTCCCATCC |
| | | AAGTACTAA |
| 5S_P3 | 189 | TTCCGAGATCAGACGAGATCGGGCGCGTTCAGGGTGGTATG |
| | | GCCGTAGAC |
| HBA1_P1 | 190 | GCCGCCCACTCAGACTTTATTCAAAGACCACGGGGGTACGG |
| | | GTGCAGGAA |
| HBA1_P2 | 191 | GGGGGAGGCCCAAGGGGCAAGAAGCATGGCCACCGAGGCTC |
| | | CAGCTTAAC |
| HBA1_P3 | 192 | GCACGGTGCTCACAGAAGCCAGGAACTTGTCCAGGGAGGCG |
| | | TGCACCGCA |
| HBA1_P4 | 193 | GGGAGGTGGGCGGCCAGGGTCACCAGCAGGCAGTGGCTTAG |
| | | GAGCTTGAA |
| HBA1_P5 | 194 | CCGAAGCTTGTGCGCGTGCAGGTCGCTCAGGGCGGACAGCG |
| | | CGTTGGGCA |
| HBA1_P6 | 195 | CCACGGCGTTGGTCAGCGCGTCGGCCACCTTCTTGCCGTGG |
| | | CCCTTAACC |
| HBA1_P7 | 196 | CTCAGGTCGAAGTGCGGGAAGTAGGTCTTGGTGGTGGGGAA |
| | | GGACAGGAA |
| HBA1_P8 | 197 | CTCCGCACCATACTCGCCAGCGTGCGCGCCGACCTTACCCC |
| | | AGGCGGCCT |
| HBA1_P9 | 198 | CGGCAGGAGACAGCACCATGGTGGGTTCTCTCTGAGTCTGT |
| | | GGGGACCAG |
| HBA2_P1 | 199 | GAGGGGAGGAGGGCCCGTTGGGAGGCCCAGCGGGCAGGAGG |
| | | AACGGCTAC |
| HBA2_P2 | 200 | ACGGTATTTGGAGGTCAGCACGGTGCTCACAGAAGCCAGGA |
| | | ACTTGTCCA |
| HBA2_P3 | 201 | CAGGGGTGAACTCGGCGGGGAGGTGGGCGGCCAGGGTCACC |
| | | AGCAGGCAG |
| HBA2_P4 | 202 | AAGTTGACCGGGTCCACCCGAAGCTTGTGCGCGTGCAGGTC |
| | | GCTCAGGGC |
| HBA2_P5 | 203 | CATGTCGTCCACGTGCGCCACGGCGTTGGTCAGCGCGTCGG |
| | | CCACCTTCT |
| HBA2_P6 | 204 | CCTGGGCAGAGCCGTGGCTCAGGTCGAAGTGCGGGAAGTAG |
| | | GTCTTGGTG |
| HBA2_P7 | 205 | AACATCCTCTCCAGGGCCTCCGCACCATACTCGCCAGCGTG |
| | | CGCGCCGAC |
| HBA2_P8 | 206 | CTTGACGTTGGTCTTGTCGGCAGGAGACAGCACCATGGTGG |
| | | GTTCTCTCT |
| HBB_P1 | 207 | GCAATGAAAATAAATGTTTTTTATTAGGCAGAATCCAGATG |
| | | CTCAAGGCC |
| HBB_P2 | 208 | CAGTTTAGTAGTTGGACTTAGGGAACAAAGGAACCTTTAAT |
| | | AGAAATTGG |
| HBB_P3 | 209 | GCTTAGTGATACTTGTGGGCCAGGGCATTAGCCACACCAGC |
| | | CACCACTTT |
| HBB_P4 | 210 | CACTGGTGGGGTGAATTCTTTGCCAAAGTGATGGGCCAGCA |
| | | CACAGACCA |
| HBB_P5 | 211 | GCCTGAAGTTCTCAGGATCCACGTGCAGCTTGTCACAGTGC |
| | | AGCTCACTC |
| HBB_P6 | 212 | CCCTTGAGGTTGTCCAGGTGAGCCAGGCCATCACTAAAGGC |
| | | ACCGAGCAC |
| HBB_P7 | 213 | CTTCACCTTAGGGTTGCCCATAACAGCATCAGGAGTGGACA |
| | | GATCCCCAA |
| HBB_P8 | 214 | TCTGGGTCCAAGGGTAGACCACCAGCAGCCTGCCCAGGGCC |
| | | TCACCACCA |
| HBB_P9 | 215 | ACCTTGCCCCACAGGGCAGTAACGGCAGACTTCTCCTCAGG |
| | | AGTCAGATG |
| HBG1_P1 | 216 | GTGATCTCTCAGCAGAATAGATTTATTATTTGTATTGCTTG |
| | | CAGAATAAA |
| HBG1_P2 | 217 | CTCTGAATCATGGGCAGTGAGCTCAGTGGTATCTGGAGGAC |
| | | AGGGCACTG |
| HBG1_P3 | 218 | ATCTTCTGCCAGGAAGCCTGCACCTCAGGGGTGAATTCTTT |
| | | GCCGAAATG |
| HBG1_P4 | 219 | CACCAGCACATTTCCCAGGAGCTTGAAGTTCTCAGGATCCA |
| | | CATGCAGCT |
| HBG1_P5 | 220 | CACTCAGCTGGGCAAAGGTGCCCTTGAGATCATCCAGGTGC |
| | | TTTGTGGCA |
| HBG1_P6 | 22 | AGCACCTTCTTGCCATGTGCCTTGACTTTGGGGTTGCCCAT |
| | | GATGGCAGA |
| HBG1_P7 | 222 | GCCAAAGCTGTCAAAGAACCTCTGGGTCCATGGGTAGACAA |
| | | CCAGGAGCC |
| HBG1_P8 | 223 | CTCCAGCATCTTCCACATTCACCTTGCCCCACAGGCTTGTG |
| | | ATAGTAGCC |
| HBG1_P9 | 224 | AAATGACCCATGGCGTCTGGACTAGGAGCTTATTGATAACC |
| | | TCAGACGTT |
| HBG2_P1 | 225 | GTGATCTCTTAGCAGAATAGATTTATTATTTGATTGCTTGC |
| | | AGAATAAAG |
| HBG2_P2 | 226 | TCTGCATCATGGGCAGTGAGCTCAGTGGTATCTGGAGGACA |
| | | GGGCACTGG |
| HBG2_P3 | 227 | TCTTCTGCCAGGAAGCCTGCACCTCAGGGGTGAATTCTTTG |
| | | CCGAAATGG |
| HBG2_P4 | 228 | ACCAGCACATTTCCCAGGAGCTTGAAGTTCTCAGGATCCAC |
| | | ATGCAGCTT |
| HBG2_P5 | 229 | ACTCAGCTGGGCAAAGGTGCCCTTGAGATCATCCAGGTGCT |
| | | TTATGGCAT |
| HBG2_P6 | 230 | GCACCTTCTTGCCATGTGCCTTGACTTTGGGGTTGCCCATG |
| | | ATGGCAGAG |
| HBG2_P7 | 231 | CCAAAGCTGTCAAAGAACCTCTGGGTCCATGGGTAGACAAC |
| | | CAGGAGCCT |
| HBG2_P8 | 232 | TCCAGCATCTTCCACATTCACCTTGCCCCACAGGCTTGTGA |
| | | TAGTAGCCT |
| HBG2_P9 | 233 | AATGACCCATGGCGTCTGGACTAGGAGCTTATTGATAACCT |
| | | CAGACGTTC |
| 5S_GNbac_P1 | 234 | ATGCCTGGCAGTTCCCTACTCTCGCATGGGGAGACCCCACA |
| | | CTACCATCG |
| 5S_GNbac_P2 | 235 | ACTTCTGAGTTCGGCATGGGGTCAGGTGGGACCACCGCGCT |
| | | ACGGCCGCC |
| 16S_GNbac_P1 | 236 | GGTTACCTTGTTACGACTTCACCCCAGTCATGAATCACAAA |
| | | GTGGTAAGT |
| 16S_GNbac_P2 | 237 | AAGCTACCTACTTCTTTTGCAACCCACTCCCATGGTGTGAC |
| | | GGGCGGTGT |
| 16S_GNbac_P3 | 238 | ACGTATTCACCGTGGCATTCTGATCCACGATTACTAGCGAT |
| | | TCCGACTTC |
| 16S_GNbac_P4 | 239 | AGACTCCAATCCGGACTACGACGCACTTTATGAGGTCCGCT |
| | | TGCTCTCGC |
| 16S_GNbac_P5 | 240 | TGTATGCGCCATTGTAGCACGTGTGTAGCCCTGGTCGTAAG |
| | | GGCCATGAT |
| 16S_GNbac_P6 | 241 | CCACCTTCCTCCAGTTTATCACTGGCAGTCTCCTTTGAGTT |
| | | CCCGGCCGG |
| 16S_GNbac_P7 | 242 | GGATAAGGGTTGCGCTCGTTGCGGGACTTAACCCAACATTT |
| | | CACAACACG |
| 16S_GNbac_P8 | 243 | TGCAGCACCTGTCTCACGGTTCCCGAAGGCACATTCTCATC |
| | | TCTGAAAAC |
| 16S_GNbac_P9 | 244 | GACCAGGTAAGGTTCTTCGCGTTGCATCGAATTAAACCACA |
| | | TGCTCCACC |
| 16S_GNbac_P10 | 245 | CGTCAATTCATTTGAGTTTTAACCTTGCGGCCGTACTCCCC |
| | | AGGCGGTCG |
| 16S_GNbac_P11 | 246 | TCCGGAAGCCACGCCTCAAGGGCACAACCTCCAAGTCGACA |
| | | TCGTTTACG |
| 16S_GNbac_P12 | 247 | GTATCTAATCCTGTTTGCTCCCCACGCTTTCGCACTGAGCG |
| | | TCAGTCTTC |
| 16S_GNbac_P13 | 248 | TTCGCCACCGGTATTCCTCCAGATCTCTACGCATTTCACCG |
| | | CTACACCTG |
| 16S_GNbac_P14 | 249 | CTACGAGACTCAAGCTTGCCAGTATCAGATGCAGTTCCCAG |
| | | GTTGAGCCC |
| 16S_GNbac_P15 | 250 | GACTTAACAAACCGCCTGCGTGCGCTTTACGCCCAGTAATT |
| | | CCGATTAAC |
| 16S_GNbac_P16 | 251 | ATTACCGCGGCTGCTGGCACGGAGTTAGCCGGTGCTTCTTC |
| | | TGCGGGTAA |
| 16S_GNbac_P17 | 252 | GTATTAACTTTACTCCCTTCCTCCCCGCTGAAAGTACTTTA |
| | | CAACCCGAA |
| 16S_GNbac_P18 | 253 | CGCGGCATGGCTGCATCAGGCTTGCGCCCATTGTGCAGTAT |
| | | TCCCCACTG |
| 16S_GNbac_P19 | 254 | GTCTGGACCGTGTCTCAGTTCCAGTGTGGCTGGTCATCCTC |
| | | TCAGACCAG |
| 16S_GNbac_P20 | 255 | TAGGTGAGCCGTTACCCCACCTACTAGCTAATCCCATCTGG |
| | | GCACATCCG |
| 16S_GNbac_P21 | 256 | AAGGTCCCCCTCTTTGGTCTTGCGACGTTATGCGGTATTAG |
| | | CTACCGTTT |
| 16S_GNbac_P22 | 257 | CTCCATCAGGCAGTTTCCCAGACATTACTCACCCGTCCGCC |
| | | ACTCGTCAG |
| 23S_GNbac_P1 | 258 | AAGGTTAAGCCTCACGGTTCATTAGTACCGGTTAGCTCAAC |
| | | GCATCGCTG |
| 23S_GNbac_P2 | 259 | CCTATCAACGTCGTCGTCTTCAACGTTCCTTCAGGACCCTT |
| | | AAAGGGTCA |
| 23S_GNbac_P3 | 260 | GGGGCAAGTTTCGTGCTTAGATGCTTTCAGCACTTATCTCT |
| | | TCCGCATTT |
| 23S_GNbac_P4 | 261 | CCATTGGCATGACAACCCGAACACCAGTGATGCGTCCACTC |
| | | CGGTCCTCT |
| 23S_GNbac_P5 | 262 | CCCCCTCAGTTCTCCAGCGCCCACGGCAGATAGGGACCGAA |
| | | CTGTCTCAC |
| 23S_GNbac_P6 | 263 | GCTCGCGTACCACTTTAAATGGCGAACAGCCATACCCTTGG |
| | | GACCTACTT |
| 23S_GNbac_P7 | 264 | ATGAGCCGACATCGAGGTGCCAAACACCGCCGTCGATATGA |
| | | ACTCTTGGG |
| 23S_GNbac_P8 | 265 | ATCCCCGGAGTACCTTTTATCCGTTGAGCGATGGCCCTTCC |
| | | ATTCAGAAC |
| 23S_GNbac_P9 | 266 | ACCTGCTTTCGCACCTGCTCGCGCCGTCACGCTCGCAGTCA |
| | | AGCTGGCTT |
| 23S_GNbac_P10 | 267 | CCTCCTGATGTCCGACCAGGATTAGCCAACCTTCGTGCTCC |
| | | TCCGTTACT |
| 23S_GNbac_P11 | 268 | GCCCCAGTCAAACTACCCACCAGACACTGTCCGCAACCCGG |
| | | ATTACGGGT |
| 23S_GNbac_P12 | 269 | AAACATTAAAGGGTGGTATTTCAAGGTCGGCTCCATGCAGA |
| | | CTGGCGTCC |
| 23S_GNbac_P13 | 270 | CCACCTATCCTACACATCAAGGCTCAATGTTCAGTGTCAAG |
| | | CTATAGTAA |
| 23S_GNbac_P14 | 271 | TTCCGTCTTGCCGCGGGTACACTGCATCTTCACAGCGAGTT |
| | | CAATTTCAC |
| 23S_GNbac_P15 | 272 | GACAGCCTGGCCATCATTACGCCATTCGTGCAGGTCGGAAC |
| | | TTACCCGAC |
| 23S_GNbac_P16 | 273 | CTTAGGACCGTTATAGTTACGGCCGCCGTTTACCGGGGCTT |
| | | CGATCAAGA |
| 23S_GNbac_P17 | 274 | ACCCCATCAATTAACCTTCCGGCACCGGGCAGGCGTCACAC |
| | | CGTATACGT |
| 23S_GNbac_P18 | 275 | CACAGTGCTGTGTTTTTAATAAACAGTTGCAGCCAGCTGGT |
| | | ATCTTCGAC |
| 23S_GNbac_P19 | 276 | CCGCGAGGGACCTCACCTACATATCAGCGTGCCTTCTCCCG |
| | | AAGTTACGG |
| 23S_GNbac_P20 | 277 | TTCCTTCACCCGAGTTCTCTCAAGCGCCTTGGTATTCTCTA |
| | | CCTGACCAC |
| 23S_GNbac_P21 | 278 | GTACGATTTGATGTTACCTGATGCTTAGAGGCTTTTCCTGG |
| | | AAGCAGGGC |
| 23S_GNbac_P22 | 279 | ACCGTAGTGCCTCGTCATCACGCCTCAGCCTTGATTTTCCG |
| | | GATTTGCCT |
| 23S_GNbac_P23 | 280 | ACGCTTAAACCGGGACAACCGTCGCCCGGCCAACATAGCCT |
| | | TCTCCGTCC |
| 23S_GNbac_P24 | 281 | ACCAAGTACAGGAATATTAACCTGTTTCCCATCGACTACGC |
| | | CTTTCGGCC |
| 23S_GNbac_P25 | 282 | ACTCACCCTGCCCCGATTAACGTTGGACAGGAACCCTTGGT |
| | | CTTCCGGCG |
| 23S_GNbac_P26 | 283 | CGCTTTATCGTTACTTATGTCAGCATTCGCACTTCTGATAC |
| | | CTCCAGCAT |
| 23S_GNbac_P27 | 284 | TTCGCAGGCTTACAGAACGCTCCCCTACCCAACAACGCATA |
| | | AGCGTCGCT |
| 23S_GNbac_P28 | 285 | CATGGTTTAGCCCCGTTACATCTTCCGCGCAGGCCGACTCG |
| | | ACCAGTGAG |
| 23S_GNbac_P29 | 286 | TAAATGATGGCTGCTTCTAAGCCAACATCCTGGCTGTCTGG |
| | | GCCTTCCCA |
| 23S_GNbac_P30 | 287 | AACCATGACTTTGGGACCTTAGCTGGCGGTCTGGGTTGTTT |
| | | CCCTCTTCA |
| 23S_GNbac_P31 | 288 | CCCGCCGTGTGTCTCCCGTGATAACATTCTCCGGTATTCGC |
| | | AGTTTGCAT |
| 23S_GNbac_P32 | 289 | GGATGACCCCCTTGCCGAAACAGTGCTCTACCCCCGGAGAT |
| | | GAATTCACG |
| 23S_GNbac_P33 | 290 | AGCTTTCGGGGAGAACCAGCTATCTCCCGGTTTGATTGGCC |
| | | TTTCACCCC |
| 23S_GNbac_P34 | 291 | CGCTAATTTTTCAACATTAGTCGGTTCGGTCCTCCAGTTAG |
| | | TGTTACCCA |
| 23S_GNbac_P35 | 292 | ATGGCTAGATCACCGGGTTTCGGGTCTATACCCTGCAACTT |
| | | AACGCCCAG |
| 23S_GNbac_P36 | 293 | CCTTCGGCTCCCCTATTCGGTTAACCTTGCTACAGAATATA |
| | | AGTCGCTGA |
| 23S_GNbac_P37 | 294 | GTACGCAGTCACACGCCTAAGCGTGCTCCCACTGCTTGTAC |
| | | GTACACGGT |
| 23S_GNbac_P38 | 295 | ACTCCCCTCGCCGGGGTTCTTTTCGCCTTTCCCTCACGGTA |
| | | CTGGTTCAC |
| 23S_GNbac_P39 | 296 | AGTATTTAGCCTTGGAGGATGGTCCCCCCATATTCAGACAG |
| | | GATACCACG |
| 23S_GNbac_P40 | 297 | ATCGAGCTCACAGCATGTGCATTTTTGTGTACGGGGCTGTC |
| | | ACCCTGTAT |
| 23S_GNbac_P41 | 298 | ACGCTTCCACTAACACACACACTGATTCAGGCTCTGGGCTG |
| | | CTCCCCGTT |
| 23S_GNbac_P42 | 299 | GGGGAATCTCGGTTGATTTCTTTTCCTCGGGGTACTTAGAT |
| | | GTTTCAGTT |
| 23S_GNbac_P43 | 300 | ATTAACCTATGGATTCAGTTAATGATAGTGTGTCGAAACAC |
| | | ACTGGGTTT |
| 23S_GNbac_P44 | 301 | GCCGGTTATAACGGTTCATATCACCTTACCGACGCTTATCG |
| | | CAGATTAGC |
| 5S_GPbac_P1 | 302 | GCTTGGCGGCGTCCTACTCTCACAGGGGGAAACCCCCGACT |
| | | ACCATCGGC |
| 5S_GPbac_P2 | 303 | TTCCGTGTTCGGTATGGGAACGGGTGTGACCTCTTCGCTAT |
| | | CGCCACCAA |
| 16S_GPbac_P1 | 304 | TAGAAAGGAGGTGATCCAGCCGCACCTTCCGATACGGCTAC |
| | | CTTGTTACG |
| 16S_GPbac_P2 | 305 | TCTGTCCCACCTTCGGCGGCTGGCTCCTAAAAGGTTACCTC |
| | | ACCGACTTC |
| 16S_GPbac_P3 | 306 | TCGTGGTGTGACGGGCGGTGTGTACAAGGCCCGGGAACGTA |
| | | TTCACCGCG |
| 16S_GPbac_P4 | 307 | ATTACTAGCGATTCCAGCTTCACGCAGTCGAGTTGCAGACT |
| | | GCGATCCGA |
| 16S_GPbac_P5 | 308 | GTGGGATTGGCTTAACCTCGCGGTTTCGCTGCCCTTTGTTC |
| | | TGTCCATTG |
| 16S_GPbac_P6 | 309 | CCAGGTCATAAGGGGCATGATGATTTGACGTCATCCCCACC |
| | | TTCCTCCGG |
| 16S_GPbac_P7 | 310 | CACCTTAGAGTGCCCAACTGAATGCTGGCAACTAAGATCAA |
| | | GGGTTGCGC |
| 16S_GPbac_P8 | 311 | ACCCAACATCTCACGACACGAGCTGACGACAACCATGCACC |
| | | ACCTGTCAC |
| 16S_GPbac_P9 | 312 | GACGTCCTATCTCTAGGATTGTCAGAGGATGTCAAGACCTG |
| | | GTAAGGTTC |
| 16S_GPbac_P10 | 313 | ATTAAACCACATGCTCCACCGCTTGTGCGGGCCCCCGTCAA |
| | | TTCCTTTGA |
| 16S_GPbac_P11 | 314 | CCGTACTCCCCAGGCGGAGTGCTTAATGCGTTAGCTGCAGC |
| | | ACTAAGGGG |
| 16S_GPbac_P12 | 315 | ACTTAGCACTCATCGTTTACGGCGTGGACTACCAGGGTATC |
| | | TAATCCTGT |
| 16S_GPbac_P13 | 316 | TCGCTCCTCAGCGTCAGTTACAGACCAGAGAGTCGCCTTCG |
| | | CCACTGGTG |
| 16S_GPbac_P14 | 317 | ACGCATTTCACCGCTACACGTGGAATTCCACTCTCCTCTTC |
| | | TGCACTCAA |
| 16S_GPbac_P15 | 318 | ATGACCCTCCCCGGTTGAGCCGGGGGCTTTCACATCAGACT |
| | | TAAGAAACC |
| 16S_GPbac_P16 | 319 | ACGCCCAATAATTCCGGACAACGCTTGCCACCTACGTATTA |
| | | CCGCGGCTG |
| 16S_GPbac_P17 | 320 | CCGTGGCTTTCTGGTTAGGTACCGTCAAGGTACCGCCCTAT |
| | | TCGAACGGT |
| 16S_GPbac_P18 | 321 | ACAACAGAGCTTTACGATCCGAAAACCTTCATCACTCACGC |
| | | GGCGTTGCT |
| 16S_GPbac_P19 | 322 | CCATTGCGGAAGATTCCCTACTGCTGCCTCCCGTAGGAGTC |
| | | TGGGCCGTG |
| 16S_GPbac_P20 | 323 | GGCCGATCACCCTCTCAGGTCGGCTACGCATCGTCGCCTTG |
| | | GTGAGCCGT |
| 16S_GPbac_P21 | 324 | CTAATGCGCCGCGGGTCCATCTGTAAGTGGTAGCCGAAGCC |
| | | ACCTTTTAT |
| 16S_GPbac_P22 | 325 | TTCAAACAACCATCCGGTATTAGCCCCGGTTTCCCGGAGTT |
| | | ATCCCAGTC |
| 16S_GPbac_P23 | 326 | CCACGTGTTACTCACCCGTCCGCCGCTAACATCAGGGAGCA |
| | | AGCTCCCAT |
| 16S_GPbac_P24 | 327 | GCATGTATTAGGCACGCCGCCAGCGTTCGTCCTGAGCCAGG |
| | | ATCAAACTC |
| 23S_GPbac_P1 | 328 | TGGTTAAGTCCTCGATCGATTAGTATCTGTCAGCTCCATGT |
| | | GTCGCCACA |
| 23S_GPbac_P2 | 329 | TATCAACCTGATCATCTTTCAGGGATCTTACTTCCTTGCGG |
| | | AATGGGAAA |
| 23S_GPbac_P3 | 330 | GGCTTCATGCTTAGATGCTTTCAGCACTTATCCCGTCCGCA |
| | | CATAGCTAC |
| 23S_GPbac_P4 | 331 | GCAGAACAACTGGTACACCAGCGGTGCGTCCATCCCGGTCC |
| | | TCTCGTACT |
| 23S_GPbac_P5 | 332 | CAAATTTCCTGCGCCCGCGACGGATAGGGACCGAACTGTCT |
| | | CACGACGTT |
| 23S_GPbac_P6 | 333 | GTACCGCTTTAATGGGCGAACAGCCCAACCCTTGGGACTGA |
| | | CTACAGCCC |
| 23S_GPbac_P7 | 334 | CGACATCGAGGTGCCAAACCTCCCCGTCGATGTGGACTCTT |
| | | GGGGGAGAT |
| 23S_GPbac_P8 | 335 | GGGGTAGCTTTTATCCGTTGAGCGATGGCCCTTCCATGCGG |
| | | AACCACCGG |
| 23S_GPbac_P9 | 336 | TTTCGTCCCTGCTCGACTTGTAGGTCTCGCAGTCAAGCTCC |
| | | CTTGTGCCT |
| 23S_GPbac_P10 | 337 | GATTTCCAACCATTCTGAGGGAACCTTTGGGCGCCTCCGTT |
| | | ACCTTTTAG |
| 23S_GPbac_P11 | 338 | GTCAAACTGCCCACCTGACACTGTCTCCCCGCCCGATAAGG |
| | | GCGGCGGGT |
| 23S_GPbac_P12 | 339 | GCCAGGGTAGTATCCCACCGATGCCTCCACCGAAGCTGGCG |
| | | CTCCGGTTT |
| 23S_GPbac_P13 | 340 | ATCCTGTACAAGCTGTACCAACATTCAATATCAGGCTGCAG |
| | | TAAAGCTCC |
| 23S_GPbac_P14 | 341 | CCTGTCGCGGGTAACCTGCATCTTCACAGGTACTATAATTT |
| | | CACCGAGTC |
| 23S_GPbac_P15 | 342 | GCCCAGATCGTTGCGCCTTTCGTGCGGGTCGGAACTTACCC |
| | | GACAAGGAA |
| 23S_GPbac_P16 | 343 | ACCGTTATAGTTACGGCCGCCGTTTACTGGGGCTTCAATTC |
| | | GCACCTTCG |
| 23S_GPbac_P17 | 344 | CCTCTTAACCTTCCAGCACCGGGCAGGCGTCAGCCCCTATA |
| | | CTTCGCCTT |
| 23S_GPbac_P18 | 345 | CCTGTGTTTTTGCTAAACAGTCGCCTGGGCCTATTCACTGC |
| | | GGCTCTCTC |
| 23S_GPbac_P19 | 346 | CAGAGCACCCCTTCTCCCGAAGTTACGGGGTCATTTTGCCG |
| | | AGTTCCTTA |
| 23S_GPbac_P20 | 347 | ATCACCTTAGGATTCTCTCCTCGCCTACCTGTGTCGGTTTG |
| | | CGGTACGGG |
| 23S_GPbac_P21 | 348 | TAGAGGCTTTTCTTGGCAGTGTGGAATCAGGAACTTCGCTA |
| | | CTATATTTC |
| 23S_GPbac_P22 | 349 | TCAGCCTTATGGGAAACGGATTTGCCTATTTCCCAGCCTAA |
| | | CTGCTTGGA |
| 23S_GPbac_P23 | 350 | CCGCGCTTACCCTATCCTCCTGCGTCCCCCCATTGCTCAAA |
| | | TGGTGAGGA |
| 23S_GPbac_P24 | 351 | TCAACCTGTTGTCCATCGCCTACGCCTTTCGGCCTCGGCTT |
| | | AGGTCCCGA |
| 23S_GPbac_P25 | 352 | CGAGCCTTCCTCAGGAAACCTTAGGCATTCGGTGGAGGGGA |
| | | TTCTCACCC |
| 23S_GPbac_P26 | 353 | TACCGGCATTCTCACTTCTAAGCGCTCCACCAGTCCTTCCG |
| | | GTCTGGCTT |
| 23S_GPbac_P27 | 354 | GCTCTCCTACCACTGTTCGAAGAACAGTCCGCAGCTTCGGT |
| | | GATACGTTT |
| 23S_GPbac_P28 | 355 | TCGGCGCAGAGTCACTCGACCAGTGAGCTATTACGCACTCT |
| | | TTAAATGGT |
| 23S_GPbac_P29 | 356 | AACATCCTGGTTGTCTAAGCAACTCCACATCCTTTTCCACT |
| | | TAACGTATA |
| 23S_GPbac_P30 | 357 | TGGCGGTCTGGGCTGTTTCCCTTTCGACTACGGATCTTATC |
| | | ACTCGCAGT |
| 23S_GPbac_P31 | 358 | AAGTCATTGGCATTCGGAGTTTGACTGAATTCGGTAACCCG |
| | | GTAGGGGCC |
| 23S_GPbac_P32 | 359 | GCTCTACCTCCAAGACTCTTACCTTGAGGCTAGCCCTAAAG |
| | | CTATTTCGG |
| 23S_GPbac_P33 | 360 | TCCAGGTTCGATTGGCATTTCACCCCTACCCACACCTCATC |
| | | CCCGCACTT |
| 23S_GPbac_P34 | 361 | TTCGGGCCTCCATTCAGTGTTACCTGAACTTCACCCTGGAC |
| | | ATGGGTAGA |
| 23S_GPbac_P35 | 362 | TCTACGACCACGTACTCATGCGCCCTATTCAGACTCGCTTT |
| | | CGCTGCGGC |
| 23S_GPbac_P36 | 363 | TAACCTTGCACGGGATCGTAACTCGCCGGTTCATTCTACAA |
| | | AAGGCACGC |
| 23S_GPbac_P37 | 364 | GGCTCTGACTACTTGTAGGCACACGGTTTCAGGATCTCTTT |
| | | CACTCCCCT |
| 23S_GPbac_P38 | 365 | ACCTTTCCCTCACGGTACTGGTTCACTATCGGTCACTAGGG |
| | | AGTATTTAG |
| 23S_GPbac_P39 | 366 | CTCCCGGATTCCGACGGAATTTCACGTGTTCCGCCGTACTC |
| | | AGGATCCAC |
| 23S_GPbac_P40 | 367 | GTTTTGACTACAGGGCTGTTACCTCCTATGGCGGGCCTTTC |
| | | CAGACCTCT |
| 23S_GPbac_P41 | 368 | CTTTGTAACTCCGTACAGAGTGTCCTACAACCCCAAGAGGC |
| | | AAGCCTCTT |
| 23S_GPbac_P42 | 369 | CGTTTCGCTCGCCGCTACTCAGGGAATCGCATTTGCTTTCT |
| | | CTTCCTCCG |
| 23S_GPbac_P43 | 370 | CAGTTCCCCGGGTCTGCCTTCTCATATCCTATGAATTCAGA |
| | | TATGGATAC |
| 23S_GPbac_P44 | 371 | GGTGGGTTTCCCCATTCGGAAATCTCCGGATCAAAGCTTGC |
| | | TTACAGCTC |
| 23S_GPbac_P45 | 372 | TGTTCGTCCCGTCCTTCATCGGCTCCTAGTGCCAAGGCATC |
| | | CACCGTGCG |
| 16S:A1 | 373 | AAACTAGATTCGAATATAACAAAACATTACATCCTCATCCA |
| | | ATCCCTTTT |
| 16S:A2 | 374 | GCGGTGTGTGCAAGGAGCAGGGACGTATTCACCGCGCGATT |
| | | GTGACACGC |
| 16S:A3 | 375 | GCCTTTCGGCGTCGGAACCCATTGTCTCAGCCATTGTAGCC |
| | | CGCGTGTTG |
| 16S:A4 | 376 | GCATACGGACCTACCGTCGTCCACTCCTTCCTCCTATTTAT |
| | | CATAGGCGG |
| 16S:A5 | 377 | CGGCATCCAAAAAAGGATCCGCTGGTAACTAAGAGCGTGGG |
| | | TCTCGCTCG |
| 16S:A6 | 378 | CAACCTGGCTATCATACAGCTGTCGCCTCTGGTGAGATGTC |
| | | CGGCGTTGA |
| 16S:A7 | 379 | AGGCTCCACGCGTTGTGGTGCTCCCCCGCCAATTCCTTTAA |
| | | GTTTCAGTC |
| 16S:A8 | 380 | CCAGGCGGCGGACTTAACAGCTTCCCTTCGGCACTGGGACA |
| | | GCTCAAAGC |
| 16S:A9 | 381 | TCCGCATCGTTTACAGCTAGGACTACCCGGGTATCTAATCC |
| | | GGTTCGCGC |
| 16S:A10 | 382 | TTCCCACAGTTAAGCTGCAGGATTTCACCAGAGACTTATTA |
| | | AACCGGCTA |
| 16S:A12 | 383 | CTCTTATTCCAAAAGCTCTTTACACTAATGAAAAGCCATCC |
| | | CGTTAAGAA |
| 16S:A13 | 384 | CCCCCGTCGCGATTTCTCACATTGCGGAGGTTTCGCGCCTG |
| | | CTGCACCCC |
| 16S:A14 | 385 | TTGTCTCAGGTTCCATCTCCGGGCTCTTGCTCTCACAACCC |
| | | GTACCGATC |
| 16S:A16 | 386 | CATTACCTAACCAACTACCTAATCGGCCGCAGACCCATCCT |
| | | TAGGCGAAA |
| 16S:A17 | 387 | AAACCATTACAGGAATAATTGCCTATCCAGTATTATCCCCA |
| | | GTTTCCCAG |
| 16S:A18 | 388 | AAGGGTAGGTTATCCACGTGTTACTGAGCCGTACGCCACGA |
| | | GCCTAAACT |
| 23S:A1 | 389 | ACCTAGCGCGTAGCTGCCCGGCACTGCCTTATCAGACAACC |
| | | GGTCGACCA |
| 23S:A2 | 390 | CGTTCCTCTCGTACTGGAGCCACCTTCCCCTCAGACTACTA |
| | | ACACATCCA |
| 23S:A3 | 391 | CCTGTCTCACGACGGTCTAAACCCAGCTCACGTTCCCCTTT |
| | | AATGGGCGA |
| 23S:A4 | 392 | GGTGCTGCTGCACACCCAGGATGGAAAGAACCGACATCGAA |
| | | GTAGCAAGC |
| 23S:A5 | 393 | GGCTCTTGCCTGCGACCACCCAGTTATCCCCGAGGTAGTTT |
| | | TTCTGTCAT |
| 23S:A6 | 394 | AGGAGGACTCTGAGGTTCGCTAGGCCCGGCTTTCGCCTCTG |
| | | GATTTCTTG |
| 23S:A7 | 395 | CAAAGTAAGTTAGAAACACAGTCATAAGAAAGTGGTGTCTC |
| | | AAGAACGAA |
| 23S:A8 | 396 | GACTTATAATCGAATTCTCCCACTTACACTGCATACCTATA |
| | | ACCAAGCTT |
| 23S:A9 | 397 | GTAAAACTCTACGGGGTCTTCGCTTCCCAATGGAAGACTCT |
| | | GGCTTGTGC |
| 23S:A10 | 398 | TCACTAAGTTCTAGCTAGGGACAGTGGGGACCTCGTTCTAC |
| | | CATTCATGC |
| 23S:A11 | 399 | CGACAAGGCATTTCGCTACCTTAAGAGGGTTATAGTTACCC |
| | | CCGCCGTTT |
| 23S:A12 | 400 | AACTGAACTCCAGCTTCACGTGCCAGCACTGGGCAGGTGTC |
| | | GCCCTCTGT |
| 23S:A13 | 401 | CTAGCAGAGAGCTATGTTTTTATTAAACAGTCGGGCCCCCC |
| | | TAGTCACTG |
| 23S:A14 | 402 | TTAAAACGCCTTAGCCTACTCAGCTAGGGGCACCTGTGACG |
| | | GATCTCGGT |
| 23S:A15 | 403 | ACAAAACTAACTCCCTTTTCAAGGACTCCATGAATCAGTTA |
| | | AACCAGTAC |
| 23S:A16 | 404 | ATAATGCCTACACCTGGTTCTCGCTATTACACCTCTCCCCA |
| | | GGCTTAAAC |
| 23S:A17 | 405 | CAATCCTACAAAACATATCTCGAAGTGTCAGAAATTAGCCC |
| | | TCAACGTCA |
| 23S:A18 | 406 | CTTTGCTGCTACTACTACCAGGATCCACATACCTGCAAGGT |
| | | CCAAAGGAA |
| 23S:A19 | 407 | CAACCCACACAGGTCGCCACTCTACACAATCACCAAAAAAA |
| | | AGGTGTTCC |
| 23S:A20 | 408 | GGATTAATTCCCGTCCATTTTAGGTGCCTCTGACCTCGATG |
| | | GGTGATCTG |
| 23S:A21 | 409 | AGGGTGGCTGCTTCTAAGCCCACCTTCCCATTGTCTTGGGC |
| | | CAAAGACTC |
| 23S:A22 | 410 | GTATTTAGGGGCCTTAACCATAGTCTGAGTTGTTTCTCTTT |
| | | CGGGACACA |
| 23S:A23 | 411 | CCTCACTCCAACCTTCTACGACGGTGACGAGTTCGGAGTTT |
| | | TACAGTACG |
| 23S:A24 | 412 | CCCTAAACGTCCAATTAGTGCTCTACCCCGCCACCAACCTC |
| | | CAGTCAGGC |
| 23S:A25 | 413 | AATAGATCGACCGGCTTCGGGTTTCAATGCTGTGATTCCAG |
| | | GCCCTATTA |
| 23S:A26 | 414 | ACAACGCTGCGGGCATATCGGTTTCCCTACGACTACAAGGA |
| | | TAAAAACCT |
| 23S:A27 | 415 | ACAAAGAACTCCCTGGCCCGTGTTTCAAGACGGACGATGCA |
| | | ACACTAGTC |
| 23S:A28 | 416 | ACAATGTTACCACTGATTCTTTCGGAAGAATTCATTCCTTA |
| | | CGCGCCACA |
| 23S:A29 | 417 | CTGGTTTCAGGTACTTTTCACCCCCCTATAGGGGTACTTTT |
| | | CAGCATTCC |
| 23S:A30 | 418 | CTCTATCGGTCTTGAGACGTATTTAGAATTGGAAGTTGATG |
| | | CCTCCCACA |
| 23S:A31 | 419 | ATCACCCTCTACGGTTCTAAAATTCCAAATAAAATTCGATT |
| | | TATCCCACG |
| 23S:A32 | 420 | TCTATACACCACATCTCCCTAATATTACTAAAAGGGATTCA |
| | | GTTTGTTCT |
| 23S:A33 | 421 | GCCGTTACTAACGACATCGCATATTGCTTTCTTTTCCTCCG |
| | | CCTACTAAG |
| 23S:A34 | 422 | GGGTTCCCAATCCTACACGGATCAACACAAAAAAAATGTGC |
| | | TAGGAAGTC |
| 5S:A1 | 423 | ACTACTGGGATCGAAACGAGACCAGGTATAACCCCCATGCT |
| | | ATGACCGCA |
| MM_16S_P10 | 424 | GCGTATGCCTGGAGAATTGGAATTCTTGTTACTCATACTAA |
| | | CAGTGTTGC |
| MM_16S_P11 | 425 | GATTAACCCAATTTTAAGTTTAGGAAGTTGGTGTAAATTAT |
| | | GGAATTAAT |
| MM_16S_P12 | 426 | AGCTTGAACGCTTTCTTTATTGGTGGCTGCTTTTAGGCCTA |
| | | CAATGGTTA |
| MM_16S_P13 | 427 | ATTATTCACTATTAAAGGTTTTTTCCGTTCCAGAAGAGCTG |
| | | TCCCTCTTT |
| MM_16S_P14 | 428 | CTTACTTTTTGATTTTGTTGTTTTTTTAGCAAGTTTAAAAT |
| | | TGAACTTAA |
| MM_16S_P15 | 429 | AACCAGCTATCACCAAGCTCGTTAGGCTTTTCACCTCTACC |
| | | TAAAAATCT |
| MM_16S_P7 | 430 | AATACTTGTAATGCTAGAGGTGATGTTTTTGGTAAACAGGC |
| | | GGGGTTCTT |
| MM_16S_P8 | 431 | TTTATCTTTTTGGATCTTTCCTTTAGGCATTCCGGTGTTGG |
| | | GTTAACAGA |
| MM_16S_P9 | 432 | TTATTTATAGTGTGATTATTGCCTATAGTCTGATTAACTAA |
| | | CAATGGTTA |
| RN_16S_P4 | 433 | AGTGATTGTAGTTGTTTATTCACTATTTAAGGTTTTTTCCT |
| | | TTTCCTAAA |
| RN_16S_P5 | 434 | TGGCTATATTTTAAGTTTACATTTTGATTTGTTGTTCTGAT |
| | | GGTAAGCTT |
| RN_16S_P6 | 435 | TTTTTTTAATCTTTCCTTAAAGCACGCCTGTGTTGGGCTAA |
| | | CGAGTTAGG |
| RN_16S_P7 | 436 | TGTTGGGTTAGTACCTATGATTCGATAATTGACAATGGTTA |
| | | TCCGGGTTG |
| RN_16S_P8 | 437 | AGGAGAATTGGTTCTTGTTACTCATATTAACAGTATTTCAT |
| | | CTATGGATC |
| RN_16S_P9 | 438 | TTTGTGATATAGGAATTTATTGAGGTTTGTGGAATTAGTGT |
| | | GTGTAAGTA |
| MM_28S_P1 | 439 | GCCGGGGAGTGGGTCTTCCGTACGCCACATTTCCCACGCCG |
| | | CGACGCGCG |
| MM_28S_P10 | 440 | ACCTCGGGCCCCCGGGCGGGGCCCTTCACCTTCATTGCGCC |
| | | ACGGCGGCT |
| MM_28S_P14 | 441 | TCGCGTCCAGAGTCGCCGCCGCCGCCGGCCCCCCGAGTGTC |
| | | CGGGCCCCC |
| MM_28S_P15 | 442 | CGCTGGTTCCTCCCGCTCCGGAACCCCCGCGGGGTTGGACC |
| | | CGCCGCCCC |
| MM_28S_P16 | 443 | CGCCGACCCCCGACCCGCCCCCCGACGGGAAGAAGGAGGGG |
| | | GGAAGAGAG |
| MM_28S_P17 | 444 | GGGACGACGGGGCCCCGCGGGGAAGAGGGGAGGGCGGGCCC |
| | | GGGCGGAAA |
| MM_28S_P18 | 445 | GGCGCCGCGCGGAAAACCGCGGCCCGGGGGGCGGACCCGGC |
| | | GGGGGAACA |
| MM_28S_P19 | 446 | CCCCCACACGCGCGGGACACGCCCGCCCGCCCCCGCCACGC |
| | | ACCTCGGGA |
| MM_28S_P2 | 447 | CACCCGCTTTGGGCTGCATTCCCAAGCAACCCGACTCCGGG |
| | | AAGACCCGA |
| MM_28S_P20 | 448 | TGGAGCGAGGCCCCGCGGGGAGGGGACCCGCGCCGGCACCC |
| | | GCCGGGCTC |
| MM_28S_P21 | 449 | CGAGGCCGGCGTGCCCCGACCCCGACGCGAGGACGGGGCCG |
| | | GGCGCCGGG |
| MM_28S_P22 | 450 | TCCCCGGAGCGGGTCGCGCCCGCCCGCACGCGCGGGACGGA |
| | | CGCTTGGCG |
| MM_28S_P23 | 451 | TCCACACGAACGTGCGTTCAACGTGACGGGCGAGAGGGCGG |
| | | CCCCCTTTC |
| MM_28S_P24 | 452 | TCCCAAGACGAACGGCTCTCCGCACCGGACCCCGGTCCCGA |
| | | CGCCCGGCG |
| MM_28S_P25 | 453 | CCGCCGCGGGGACGACGCGGGGACCCCGCCGAGCGGGGACG |
| | | GACGGGGAC |
| MM_28S_P3 | 454 | GCACCGCCACGGTGGAAGTGCGCCCGGCGGCGGCCGGTCGC |
| | | CGGCCGGGG |
| MM_28S_P6 | 455 | CCCACCGGGCCCCGAGAGAGGCGACGGAGGGGGGTGGGAGA |
| | | GCGGTCGCG |
| MM_28S_P7 | 456 | CCCGGCCCCCACCCCCACGCCCGCCCGGGAGGCGGACGGGG |
| | | GGAGAGGGA |
| MM_28S_P8 | 457 | TATCTGGCTTCCTCGGCCCCGGGATTCGGCGAAAGCGCGGC |
| | | CGGAGGGCT |
| MM_28S_P9 | 458 | CGCCGCCGACCCCGTGCGCTCGGCTTCGTCGGGAGACGCGT |
| | | GACCGACGG |
| RN_28S_P12 | 459 | GCGCCCCCCCGCACCCGCCCCGTCCCCCCCGCGGACGGGGA |
| | | AGAAGGGAG |
| RN_28S_P14 | 460 | CGAACCCCGGGAACCCCCGACCCCGCGGAGGGGGAAGGGGG |
| | | AGGACGAGG |
| RN_28S_P16 | 461 | CACCCGGGGGGGCGACGAGGCGGGGACCCGCCGGACGGGGA |
| | | CGGACGGGG |
| RN_28S_P17 | 462 | GCCAACCGAGGCTCCTTCGGCGCTGCCGTATCGTTCCGCTT |
| | | GGGCGGATT |
| RN_28S_P4 | 463 | CCCGGGCCCCCGGACCCCCGAGAGGGACGACGGAGGCGACG |
| | | GGGGGTGGG |
| RN_28S_P5 | 464 | TGGGAGGGGCGGCCCGGCCCCCGCGACCGCCCCCCTTTCCG |
| | | CCACCCCAC |
| RN_28S_P6 | 465 | GGGAGAGGCCGGGGGGAGAGCGCGGCGACGGGTATCCGGCT |
| | | CCCTCGGCC |
| RN_28S_P7 | 466 | CGCTGCTGCCGGGGGGCTGTAACACTCGGGGGGGGTGGTC |
| | | CGGCGCCCA |
| RN_28S_P8 | 467 | CGCCGCCGACCCCGTGCGCTCGGCTTCGCTCCCCCCCACCC |
| | | CGAGAAGGG |

### BRIEF DESCRIPTION OF THE FIGURES

FIGS. 1A-B show an exemplary workflow for performing depletion of RNA species from a sample. In FIG. 1A, step 1 includes nucleic acid denaturation followed by addition of depletion DNA probes and hybridization of the probes with the off-target RNA species, thereby creating DNA:RNA hybrids. Step 2 includes digestion of the RNA from the DNA:RNA hybrids using a ribonuclease such as RNase H. Step 3 includes digesting residual DNA probes from the degraded mixture by addition of DNase. Step 4 includes capturing the remaining target RNA in the sample, which is optionally followed by additional manipulations that will eventually result in a sample depleted of off-target RNA species that can be sequenced, exposed to microarray expression analysis, qPCR, or other analysis techniques. FIG. 1B shows the impact of these steps schematically on nucleic acids in the sample, including messenger RNA (mRNA), small noncoding RNA (small RNA), and long noncoding RNA (Inc RNA).

FIG. 2 shows that in an Integrative Genomics Viewer (IGV) plot for one sample, there are almost 1.5 million reads stacked at one position and that this peak accounts for 17.4% of the total reads.

FIG. 3 shows analysis of focal peaks in 95 Rare and Undiagnosed Genetic Diseases (RUGD) samples. This figure shows that 9 samples has more than 10% of reads mapping to focal peaks, with two additional samples having nearly 10% of reads mapping to focal peaks.

FIG. 4 shows the proportion of reads mapping to 6 focal peaks comparing standard preparation methods and an sncRNA depletion protocol.

FIG. 5 shows another view of FIG. 2, after the sample was analyzed after a modified sncRNA depletion protocol library preparation.

FIGS. 6A-D show key library metrics, comparing values for a standard protocol to an sncRNA depletion protocol.

FIGS. 7A-H show various gene coverage relating metrics, comparing values for a standard protocol to an sncRNA depletion protocol.

FIGS. 8A-K illustrate distribution of transcripts per million (TPMs) corrected for read depth and length of gene for standard and sncRNA depletion preparations.

FIGS. 9A-K also illustrate distribution of transcripts per million (TPMs) corrected for read depth and length of gene for standard and sncRNA depletion preparations, with housekeeping genes separately identified.

FIGS. 10A-F illustrate per gene log 2 (TPM+1) of depleted and non-depleted sequencing on the same samples.

FIG. 11 illustrates the proportion of reads mapping to each focal peak gene for samples with no probes, old probes or the new probes for sncRNA depletion.

### DETAILED DESCRIPTION

### I. Off Target RNA

Described herein are methods for depleting off-target RNA molecules from a nucleic acid sample.

As used herein, the term “nucleic acid” is intended to be consistent with its use in the art and includes naturally occurring nucleic acids or functional analogs thereof. Particularly useful functional analogs are capable of hybridizing to a nucleic acid in a sequence specific fashion or capable of being used as a template for replication of a particular nucleotide sequence. Naturally occurring nucleic acids generally have a backbone containing phosphodiester bonds. An analog structure can have an alternate backbone linkage including any of a variety of those known in the art. Naturally occurring nucleic acids generally have a deoxyribose sugar (e.g., found in deoxyribonucleic acid (DNA)) or a ribose sugar (e.g., found in ribonucleic acid (RNA)). A nucleic acid can contain any of a variety of analogs of these sugar moieties that are known in the art. A nucleic acid can include native or non-native bases. In this regard, a native deoxyribonucleic acid can have one or more bases selected from the group consisting of adenine, thymine, cytosine or guanine and a ribonucleic acid can have one or more bases selected from the group consisting of uracil, adenine, cytosine or guanine. Useful non-native bases that can be included in a nucleic acid are known in the art. The term “target,” when used in reference to a nucleic acid, is intended as a semantic identifier for the nucleic acid in the context of a method or composition set forth herein and does not necessarily limit the structure or function of the nucleic acid beyond what is otherwise explicitly indicated.

In some embodiments, the present methods decrease library preparation costs and hands-on-time, as compared to prior art methods of depleting off-target RNA, followed by library preparation.

Also described herein are compositions comprising a probe set comprising at least two DNA probes complementary to discontiguous sequences at least 5, or at least 10, or 15 bases apart along the full length of at least one off-target RNA molecule in a nucleic acid sample and a ribonuclease capable of degrading RNA in a DNA:RNA hybrid, wherein the off-target RNA comprises at least one small noncoding RNA chosen from RN7SK, RN7SL1, RN7SL2, RN7SL5P, RPPH1, SNORD3A.

As used herein, “off-target RNA,” “an off-target RNA sequence”, “unwanted RNA,” or “an unwanted RNA sequence” refers to any RNA that a user does not wish to analyze. As used herein, an unwanted RNA includes the complement of an unwanted RNA sequence. When RNA is converted into cDNA and this cDNA is prepared into a library, a user would sequence library fragments that were prepared from all RNA transcripts in the absence of depletion. Methods described herein for depleting library fragments prepared from unwanted RNA can thus save the user time and consumables related to sequencing and analyzing sequencing data prepared from unwanted RNA. In some embodiments, off-target RNA relates to small non-coding RNA (sncRNA). In some embodiments, the off-target RNA comprises sncRNA with MALAT1. In some embodiments, the off-target RNA for depletion does not include MALAT. In some embodiments, off-target RNA comprises at least one small noncoding RNA chosen from RN7SK, RN7SL1, RN7SL2, RN7SL5P, RPPH1, SNORD3A. In some embodiments the off-target RNA is not MALAT1.Small noncoding RNAs are highly abundant as reads during the sequencing process and can lead to noise when analyzing sequencing data. MALAT1 is also highly abundant in the genome. MALAT1 is a highly conserved large, infrequently spliced non-coding RNA which is highly expressed in the nucleus. Trying to remove these reads during analysis after sequencing results in wasted sequencing.

As used herein, “off-target RNA,” “unwanted RNA” or “unwanted RNA sequence” also includes fragments of such RNA. For example, an unwanted RNA may comprise part of the sequence of an unwanted RNA. In some embodiments, unwanted RNA sequence is from human, rat, mouse, or bacteria. In some embodiments, the bacteria are Archaea species, E. coli, or B. subtilis.

As used herein, “off-target library fragments” or “unwanted library fragments” also includes library fragments prepared from cDNA prepared from unwanted RNA.

### A. High Abundance RNA

In some embodiments, the off-target RNA is high-abundance RNA. High-abundance RNA is RNA that is very abundant in many samples and which users do not wish to sequence, but it may or may not be present in a given sample. In some embodiments, the high-abundance RNA sequence is a ribosomal RNA (rRNA) sequence. Exemplary high-abundance RNA are disclosed in WO2021/127191 and WO 2020/132304, each of which is incorporated by reference herein in its entirety.

In some embodiments, the high-abundance RNA sequences are the most abundant RNA sequences determined to be in a sample. In some embodiments, the high-abundance RNA sequences are the most abundant RNA sequences across a plurality of samples even though they may not be the most abundant in a given sample. In some embodiments, a user utilizes a method of determining the most abundant RNA sequences in a sample, as described herein.

In a given sample, the most abundant sequences are the 100 most abundant sequences. In some embodiments, in addition to depleting the 100 most abundant sequences, the method also is capable of depleting the 1,000 most abundant sequences, or the 10,000 most abundant sequences in a sample. In some embodiments, the off-target RNA sequence comprises a sequence with homology of at least 90%, at least 95%, or at least 99% to a most abundant sequence in a sample comprising RNA. In some embodiments, the off-target RNA sequence comprises a sequence with homology of at least 90%, at least 95%, or at least 99% to a most abundant sequence in a sample comprising RNA, wherein the most abundant sequences comprise the 100 most abundant sequences. In some embodiments, homology is measured against the 1,000 most abundant sequences, or the 10,000 most abundant sequences.

In some embodiments, the high-abundance RNA sequences are comprised in RNA known to be highly abundant in a range of samples.

In some embodiments, the off-target RNA sequence is globin mRNA or 28S, 23S, 18S, 5.8S, 5S, 16S, 12S, HBA-A1, HBA-A2, HBB, HBB-B1, HBB-B2, HBG1, or HBG2 RNA, or a fragment thereof.

In some embodiments, the off-target RNA sequence is 28S, 18S, 5.8S, 5S, 16S, or 12S RNA from humans, or a fragment thereof. In some embodiments, the off-target RNA sequence is rat 16S, rat 28S, mouse 16S, or mouse 28S RNA.

In some embodiments, the off-target RNA sequence is comprised in mRNA related to one or more “housekeeping” genes. For example, a housekeeping gene may be one that is commonly expressed in a sample from a tumor or other oncology-related sample, but that is not implicated in tumor genesis or progression. Housekeeping genes are typically constitutive genes that are required for the maintenance of basal cellular functions that are essential for the existence of a cell, regardless of its specific role in the tissue or organism.

In some embodiments, the off-target RNA sequence is comprised in 23S, 16S, or 5S RNA from Gram-positive or Gram-negative bacteria.

### B. Desired RNA

As used herein, “desired RNA” or “a desired RNA sequence” refers to any RNA that a user wants to analyze. As used herein, a desired RNA includes the complement of a desired RNA sequence. Desired RNA may be RNA from which a user would like to collect sequencing data, after cDNA and library preparation. In some instances, the desired RNA is mRNA (or messenger RNA). In some instances, the desired RNA is a portion of the mRNA in a sample. For example, a user may want to analyze RNA transcribed from cancer-related genes, and thus this is the desired RNA.

As used herein, “desired library fragments” refers to library fragments prepared from cDNA prepared from desired RNA.

In some embodiments, the desired RNA sequence is an exome sequence.

In some embodiments, the desired RNA sequence is from human, rat, mouse, and/or bacteria.

### II. Compositions

Described herein is a composition comprising a probe set comprising at least two DNA probes complementary to discontiguous sequences at least 5, or at least 10, or 15 bases apart along the full length of at least one off-target RNA molecule in a nucleic acid sample and a ribonuclease capable of degrading RNA in a DNA:RNA hybrid. In some embodiments, the off-target RNA comprises at least one small noncoding RNA chosen from RN7SK, RN7SL1, RN7SL2, RN7SL5P, RPPH1, SNORD3A.

In some embodiments, at least one small noncoding RNA sequence is chosen from SEQ ID NOS: 1-6.

In some embodiments, at least one off-target RNA is chosen from the portion of SNORD3A that does not correspond to ALU.

In some embodiments, the off-target RNA is not MALAT1.

In some embodiments, the ribonuclease is RNase H.

In some embodiments, each DNA probe is hybridized at least 10 bases apart along the full length of the at least one off-target RNA molecule from any other DNA probe in the probe set.

In some embodiments, the composition comprises a destabilizing chemical.

In some embodiments, the destabilizing chemical is formamide.

In some embodiments, the off-target RNA further comprises rRNA, mRNA, tRNA, or a mixture thereof.

In some embodiments, the off-target RNA is sncRNA, rRNA, and globin mRNA.

In some embodiments, the probe set further comprises at least two DNA probes that hybridize to at least one off-target RNA molecule selected from 28S, 23S, 18S, 5.8S, 5S, 16S, 12S, HBA-A1, HBA-A2, HBB, HBG1, and HBG2.

In some embodiments, the probe set further comprises at least two DNA probes that hybridize to two or more off-target RNA molecules selected from 28S, 18S, 5.8S, 5S, 16S, and 12S from humans.

In some embodiments, the probe set further comprises at least two DNA probes that hybridize to one or more off-target RNA molecules selected from HBA-A1, HBA-A2, HBB, HBG1, and HBG2 from hemoglobin, and 23S, 16S, and 5S from Gram positive or Gram negative bacteria.

In some embodiments, the probe set further comprises at least two DNA probes complementary to one or more rRNA molecules from an Archaea species.

In some embodiments, the probe set further comprises DNA probes that hybridize to one or more off-target RNA molecules from rat and/or mouse, optionally selected from rat 16S, rat 28S, mouse 16S, and mouse 28S, and combinations thereof.

In some embodiments, the probe length is from 20 to 100 nucleotides. In some embodiments, the probe length is from 40 to 60 nucleotides. In some embodiments, the probe length is from 40 to 50 nucleotides. In some embodiments, the probe length is from 20 to 30 nucleotides. In some embodiments, the probe length is from 30 to 40 nucleotides. In some embodiments, the probe length is from 50 to 60 nucleotides. In some embodiments, the probe length is from 60 to 70 nucleotides. In some embodiments, the probe length is from 70 to 80 nucleotides. In some embodiments, the probe length is from 80 to 90 nucleotides. In some embodiments, the probe length is from 90 to 100 nucleotides.

In some embodiments, at least two probes in the probe set comprise any one of SEQ ID NOs: 8-39. In some embodiments, at least three probes in the probe set comprise any one of SEQ ID NOs: 8-39. In some embodiments, at least four probes in the probe set comprise any one of SEQ ID NOs: 8-39.

In some embodiments, the DNA probes comprise two or more, or five or more, or 10 or more, or 25 or more sequences, or all of the sequences selected from SEQ ID NOs: 8-39.

In some embodiments, the DNA probes further comprise any one of SEQ ID NOS: 40-467.

In some embodiments, the DNA probes further comprise five or more, or 10 or more, or 25 or more sequences, or all of the sequences selected from SEQ ID NOs: 40-467. In some embodiments, the probe set comprises 15 or more, 30 or more, 50 or more, 75 or more, 100 or more, 125 or more, 150 or more, 175 or more, 200 or more, 225 or more, 250 or more, 275 or more, 300 or more, 325 or more, 350 or more, 375 or more, 400 or more, or 425 or more, or more sequences, or all of the sequences selected from SEQ ID NOs: 40-467.

In some embodiments, the probe set comprises at least 10, at least 50, at least 100, 2 at least 00, at least 300, or at least 400 sequences selected from SEQ ID NOs: 40-467.

In some embodiments, the probe set comprises: (a) two or more, or five or more, or 10 or more, or 25 or more, or 50 or more, or 100 or more, or 150 or more, or 200 or more, or 250 or more, or 300 or more, or 333 sequences selected from SEQ ID NOs: 40-372; or (b) two or more, or five or more, or 10 or more, or 25 or more, or 50 or more, or 100 or more, or 150 or more, or 200 or more, or 250 or more, or 300 or more, or 350 or more, or 400 or more, or 428 sequences selected from SEQ ID NOs: 40-467; or (c) two or more, or five or more, or 10 or more, or 25 or more, or 50 or more, or 100 or more, or 150 or more, or 200 or more, or 250 or more, or 300 or more, or 350 or more, or 377 sequences selected from SEQ ID NOs: 40-416; or (d) two or more, or five or more, or 10 or more, or 25 or more, or 50 or more, or 100 or more, or 150 or more, or 200 or more, or 250 or more, or 300 or more, or 350 or more, or 384 sequences selected from SEQ ID NOs: 40-372 and SEQ ID NOs: 416-467; or (c) two or more, or five or more, or 10 or more, or 25 or more, or 44 sequences selected from SEQ ID NOs: 41-416; or (f) two or more, or five or more, or 10 or more, or 25 or more, or 50 or more, or 51 sequences selected from SEQ ID NOs: 416-467; or a combination thereof.

In some embodiments, probe set comprises sequences selected from SEQ ID NOS: 40-372, sequences selected from SEQ ID NOs: 424-32, sequences selected from SEQ ID NOs: 439-458, sequences selected from SEQ ID NOs: 433-438, and/or sequences selected from SEQ ID NOs: 459-467.

In some embodiments, the probe set further comprises at least two DNA probes that hybridize to at least one off-target RNA molecule selected from 28S, 23S, 18S, 5.8S, 5S, 16S, 12S, HBA-A1, HBA-A2, HBB, HBB-B1, HBB-B2, HBG1, and HBG2.

In some embodiments, the probe set further comprises at least two DNA probes that hybridize to two or more off-target RNA molecules selected from 28S, 18S, 5.8S, 5S, 16S, and 12S from humans.

In some embodiments, the probe set further comprises at least two DNA probes that hybridize to one or more off-target RNA molecules selected from HBA-A1, HBA-A2, HBB, HBG1, and HBG2 from hemoglobin, and 23S, 16S, and 5S from Gram positive or Gram negative bacteria.

In some embodiments, the probe set further comprises at least two DNA probes that hybridize to one or more off-target RNA molecules from an Archaea species.

### III. Methods of Use

### A. Methods of Depleting Off-Target RNA

Described herein are methods of depleting off-target library fragments, wherein the library fragments are prepared from a sample comprising RNA.

In some embodiments, the present methods decrease library preparation costs and hands-on-time, as compared to prior art methods of depleting off-target RNA, followed by library preparation.

Described herein are methods for depleting off-target RNA molecules from a nucleic acid sample. In some embodiments, the method comprises providing any of the compositions described herein, in Section II above.

In some embodiments, the method comprises providing a probe set comprising at least two DNA probes complementary to discontiguous sequences along the full length of the at least one off-target RNA molecule, wherein the at least one off-target RNA molecule comprises at least one small noncoding RNA chosen from RN7SK, RN7SL1, RN7SL2, RN7SL5P, RPPH1, SNORD3A; contacting a nucleic acid sample comprising at least one target RNA or DNA sequence and at least one off-target RNA molecule with the probe set, thereby hybridizing the DNA probes to the at least one off-target RNA molecule to form DNA:RNA hybrids, wherein each DNA:RNA hybrid is at least 5 bases apart, or at least 10 bases apart, along a given off-target RNA molecule sequence from any other DNA:RNA hybrid; and contacting the DNA:RNA hybrids with a ribonuclease that degrades the RNA from the DNA:RNA hybrids, thereby degrading the off-target RNA molecules in the nucleic acid sample to form a degraded mixture.

In some embodiments, the nucleic acid sample is an FFPE sample.

In some embodiments, the probes bind to noncoding RNA molecules leaving a 15 base pair gap between probes.

In some embodiments, the method further comprises degrading any remaining DNA probes by contacting the degraded mixture with a DNA digesting enzyme, optionally wherein the DNA digesting enzyme is DNase I, to form a DNA degraded mixture; and separating the degraded RNA from the degraded mixture or the DNA degraded mixture.

In some embodiments, the contacting with the probe set comprises treating the nucleic acid sample with a destabilizer.

In some embodiments, with the destabilizer is heat and/or a nucleic acid destabilizing chemical.

In some embodiments, the nucleic acid destabilizing chemical is betaine, DMSO, formamide, glycerol, or a derivative thereof, or a mixture thereof.

In some embodiments, the nucleic acid destabilizing chemical is formamide, optionally wherein the formamide is present during the contacting with the probe set at a concentration of from about 10 to 45% by volume.

In some embodiments, treating the sample with heat comprises applying heat above the melting temperature of the at least one DNA:RNA hybrid.

In some embodiments, the ribonuclease is RNase H or Hybridase.

In some embodiments, the nucleic acid sample is from a human

In some embodiments, the nucleic acid sample further comprises nucleic acids of non-human origin.

In some embodiments, the nucleic acids of non-human origin are from non-human eukaryotes, bacteria, viruses, plants, soil, or a mixture thereof.

In some embodiments, the off-target RNA further comprises rRNA, mRNA, tRNA, or a mixture thereof.

In some embodiments, the off-target RNA is sncRNA, rRNA, and globin mRNA.

In some embodiments, the globin mRNA is hemoglobin mRNA.

### B. Methods of Supplementing a Probe Set for Use in Depleting Off-Target RNA

Also described herein are methods of supplementing a probe set for use in depleting off-target RNA nucleic acid molecules from a nucleic acid sample.

Described herein are methods of depleting off-target library fragments wherein the library fragments are prepared from a sample comprising RNA.

The present methods of depleting are flexible for use with any upstream methods of library preparation that a user prefers. In other words, a user can choose the best method of preparation and the best method of library preparation for their particular sample, and then the user can deplete off-target RNA nucleic acid molecules using methods described herein.

In some embodiments, the method of supplementing a probe set for use in depleting off-target RNA nucleic acid molecules from a nucleic acid sample comprises: (a) contacting a nucleic acid sample comprising at least one RNA or DNA target sequence and at least one off-target RNA molecule from a first species with a probe set comprising at least two DNA probes complementary to discontiguous sequences along the full length of the at least one off-target RNA molecule from a second species, thereby hybridizing the DNA probes to the off-target RNA molecules to form DNA:RNA hybrids, wherein each DNA:RNA hybrid is at least 5 bases apart, or at least 10 bases apart, along a given off-target RNA molecule sequence from any other DNA:RNA hybrid, wherein the off-target DNA comprises at least one small noncoding RNA chosen from RN7SK, RN7SL1, RN7SL2, RN7SL5P, RPPH1, SNORD3A; (b) contacting the DNA:RNA hybrids with a ribonuclease that degrades the RNA from the DNA:RNA hybrids, thereby degrading the off-target RNA molecules in the nucleic acid sample to form a degraded mixture; (c) separating the degraded RNA from the degraded mixture; (d) sequencing the remaining RNA from the sample; (c) evaluating the remaining RNA sequences for the presence of off-target RNA molecules from the first species, thereby determining gap sequence regions; and (f) supplementing the probe set with additional DNA probes complementary to discontiguous sequences in one or more of the gap sequence regions.

In some embodiments, the first species is a non-human species and the second species is human.

In some embodiments, the first species is rat or mouse.

In some embodiments, a composition described herein is used to supply the ribonuclease and the probe set comprising DNA probes complementary to discontiguous sequences along the full length of the at least one off-target RNA molecule of a human.

In some embodiments, the method is used to identify DNA probes that hybridize to one or more off-target RNA molecules from rat and/or mouse, optionally selected from rat 16S, rat 28S, mouse 16S, and mouse 28S, small noncoding RNA, and combinations thereof.

### C. Samples

In some embodiments, the sample comprises a microbe sample, a microbiome sample, a bacteria sample, a yeast sample, a plant sample, an animal sample, a patient sample, an epidemiology sample, an environmental sample, a soil sample, a water sample, a metatranscriptomics sample, or a combination thereof.

In some embodiments, the sample may be from a mammal. In some embodiments the sample may be from a human, monkey, rat and/or mouse.

In some embodiments, samples may be from a patient. In some embodiments, samples may be from a patient with cancer (i.e., an oncology sample). In some embodiments, samples may be from a patient with a rare disease. In some embodiments, samples may be from a patient with coronavirus SARS-CoV2 (COVID-19).

In some embodiments, the sample may be a tumor sample. In some embodiments, the sample may be a blood sample. In some embodiments the sample may be a tissue sample.

For example, oncology samples may be used to evaluate changes in RNA expression in tumor cells, and to potentially monitor these changes over time or over the course of a therapeutic treatment. In such cases, RNA related to tumor markers may be desired RNA. Oncology samples may be depleted of unwanted or off target genes that are not implicated in tumorigenesis or progression.

### D. Library Preparations

Libraries prepared by any method can be used together with the present methods of depleting. In some embodiments, probes are single-stranded to allow for hybridizing and capturing of single-stranded library fragments that are complementary. In some embodiments, specific binding of a single-stranded library fragment to a probe generates a double-stranded oligonucleotide. In some embodiments, the double-stranded oligonucleotide forms a DNA:RNA hybrid. The probe specifically bound to the library fragment may be bound with a high-enough affinity to be recognized for degradation with a ribonuclease. In some embodiments, the off-target RNA molecules are degraded after contacting the sample with a ribonuclease to form a degraded mixture.

As used herein, the term “library” refers to a collection of members. In one embodiment, the library includes a collection of nucleic acid members, for example, a collection of whole genomic, subgenomic fragments, cDNA, cDNA fragments, RNA, RNA fragments, or a combination thereof. In some embodiments, a portion or all library members include a non-target adaptor sequence. The adaptor sequence can be located at one or both ends. The adaptor sequence can be used in, for example, a sequencing method (for example, an NGS method), for amplification, for reverse transcription, or for cloning into a vector.

In some embodiments, this DNA:RNA hybrid-specific cleavage is comprises use of RNase H. This methodology is implemented as part of the current Illumina Total RNA Stranded Library Prep workflow and New England Biolabs NEBNext rRNA Depletion Kit and RNA depletion methods as described in U.S. Pat. Nos. 9,745,570 and 9,005,891.

### E. Amplifying

In some embodiments, methods described herein comprise one or more amplification step. In some embodiments, library fragments are amplified before being added to a solid support. In some embodiments library fragments are amplified after a method of depleting described herein. In some embodiments, amplifying is by PCR amplification.

As used herein, “amplify,” “amplifying,” or “amplification reaction” and their derivatives, refer generally to any action or process whereby at least a portion of a nucleic acid molecule is replicated or copied into at least one additional nucleic acid molecule. The additional nucleic acid molecule optionally includes sequence that is substantially identical or substantially complementary to at least some portion of the template nucleic acid molecule. The template nucleic acid molecule can be single-stranded or double-stranded and the additional nucleic acid molecule can independently be single-stranded or double-stranded. Amplification optionally includes linear or exponential replication of a nucleic acid molecule. In some embodiments, such amplification can be performed using isothermal conditions; in other embodiments, such amplification can include thermocycling. In some embodiments, the amplification is a multiplex amplification that includes the simultaneous amplification of a plurality of target sequences in a single amplification reaction. In some embodiments, “amplification” includes amplification of at least some portion of DNA and RNA based nucleic acids alone, or in combination. The amplification reaction can include any of the amplification processes known to one of ordinary skill in the art. In some embodiments, the amplification reaction includes polymerase chain reaction (PCR).

### 1. Amplification After Depleting

In some embodiments, collected library fragments are amplified after a method of depleting. In some embodiments, a depleted library is amplified.

In some embodiments, the amplifying is performed with a thermocycler. In some embodiments, the amplifying is by PCR amplification.

As used herein, the term “polymerase chain reaction” (“PCR”) refers to the method as described in U.S. Pat. Nos. 4,683,195 and 4,683,202, which describe a method for increasing the concentration of a segment of a polynucleotide of interest in a mixture of genomic DNA without cloning or purification. This process for amplifying the polynucleotide of interest consists of introducing a large excess of two oligonucleotide primers to the DNA mixture containing the desired polynucleotide of interest, followed by a series of thermal cycling in the presence of a DNA polymerase. The two primers are complementary to their respective strands of the double stranded polynucleotide of interest. The mixture is denatured at a higher temperature first and the primers are then annealed to complementary sequences within the polynucleotide of interest molecule. Following annealing, the primers are extended with a polymerase to form a new pair of complementary strands. The steps of denaturation, primer annealing, and polymerase extension can be repeated many times (referred to as thermocycling) to obtain a high concentration of an amplified segment of the desired polynucleotide of interest. The length of the amplified segment of the desired polynucleotide of interest (amplicon) is determined by the relative positions of the primers with respect to each other, and therefore, this length is a controllable parameter. By virtue of repeating the process, the method is referred to as the “polymerase chain reaction” (hereinafter “PCR”). Because the desired amplified segments of the polynucleotide of interest become the predominant nucleic acid sequences (in terms of concentration) in the mixture, they are said to be “PCR amplified.” In a modification to the method discussed above, the target nucleic acid molecules can be PCR amplified using a plurality of different primer pairs, in some cases, one or more primer pairs per target nucleic acid molecule of interest, thereby forming a multiplex PCR reaction.

In some embodiments, the amplifying is performed without PCR amplification. In some embodiments, the amplifying does not require a thermocycler. In some embodiments, depleting and amplifying after the depleting is performed in a sequencer.

In some embodiments, the amplifying is performed without a thermocycler. In some embodiments, the amplifying is performed by bridge or cluster amplification.

### F. Sequencing of Depleted Libraries

In some embodiments, a library depleted of off-target library fragments is sequenced.

After methods of depleting described herein, the collected library may comprise less than 15%, 13%, 11%, 9%, 7%, 5%, 3%, 2% or 1% or any range in between of off-target RNA species. In some embodiments, the collected library after depleting comprises at least 99%, 98%, 97%, 95%, 93%, 91%, 89% or 87% or any range in between of desired RNA. In other words, the library for sequencing after the depleting mainly comprises library fragments that were prepared from RNA of interest.

In some embodiments, sequencing data generated after depleting of off-target library fragments has fewer sequences corresponding to off-target RNA as compared to the same library sequenced without the depleting.

Depleted libraries prepared by the present method can be used with any type of RNA sequencing, such as RNA-seq, small RNA sequencing, long non-coding RNA (lncRNA) sequencing, circular RNA (circRNA) sequencing, targeted RNA sequencing, exosomal RNA sequencing, and degradome sequencing.

Depleted libraries can be sequenced according to any suitable sequencing methodology, such as direct sequencing, including sequencing by synthesis, sequencing by ligation, sequencing by hybridization, nanopore sequencing and the like. In some embodiments, the depleted libraries are sequenced on a solid support. In some embodiments, the solid support for sequencing is the same solid support on which the depleting is performed. In some embodiments, the solid support for sequencing is the same solid support upon which amplification occurs after the depleting.

Flowcells provide a convenient solid support for performing sequencing. One or more library fragments (or amplicons produced from library fragments) in such a format can be subjected to an SBS or other detection technique that involves repeated delivery of reagents in cycles. For example, to initiate a first SBS cycle, one or more labeled nucleotides, DNA polymerase, etc., can be flowed into/through a flowcell that houses one or more amplified nucleic acid molecules. Those sites where primer extension causes a labeled nucleotide to be incorporated can be detected. Optionally, the nucleotides can further include a reversible termination property that terminates further primer extension once a nucleotide has been added to a primer. For example, a nucleotide analog having a reversible terminator moiety can be added to a primer such that subsequent extension cannot occur until a deblocking agent is delivered to remove the moiety. Thus, for embodiments that use reversible termination, a deblocking reagent can be delivered to the flowcell (before or after detection occurs). Washes can be carried out between the various delivery steps. The cycle can then be repeated n times to extend the primer by n nucleotides, thereby detecting a sequence of length n. Exemplary SBS procedures, fluidic systems and detection platforms that can be readily adapted for use with amplicons produced by the methods of the present disclosure are described, for example, in Bentley et al., Nature 456:53-59 (2008), WO 04/018497; U.S. Pat. No. 7,057,026; WO 91/06678; WO 07/123744; U.S. Pat. Nos. 7,329,492; 7,211,414; 7,315,019; 7,405,281, and US 2008/0108082, each of which is incorporated herein by reference.

The term “flow cell” as used herein refers to a chamber comprising a solid surface across which one or more fluid reagents can be flowed. Examples of flow cells and related fluidic systems and detection platforms that can be readily used in the methods of the present disclosure are described, for example, in Bentley et al., Nature 456:53-59 (2008); WO 04/018497; WO 91/06678; WO 07/123744; U.S. Pat. Nos. 7,057,026; 7,211,414; 7,315,019; 7,329,492; 7,405,281; and US Pat. Publication No. 2008/0108082.

### IV. Kits

Described herein is a kit comprising any of the compositions described herein in Section II above.

In some embodiments, the kit comprises a buffer and nucleic acid purification medium.

In some embodiments, the kit further comprises a destabilizing chemical.

In some embodiments, the kit comprises (a) a probe set comprising SEQ ID NOs: 8-39 and 40-467; (b) a ribonuclease, optionally wherein the ribonuclease is RNase H; (c) a DNase; and (d) RNA purification beads.

In some embodiments, the kit further comprises an RNA depletion buffer, a probe depletion buffer, and a probe removal buffer.

Throughout this application and claims, the term “and/or” means one or more of the listed elements or a combination of any two or more of the listed elements.

The term “comprises” and variations thereof do not have a limiting meaning where these terms appear in the description and claims.

It is understood that wherever embodiments are described herein with the language “include,” “includes,” or “including,” and the like, otherwise analogous embodiments described in terms of “consisting of” and/or “consisting essentially of” are also provided. The term “consisting of” is limited to whatever follows the phrase “consisting of.” That is, “consisting of” indicates that the listed elements are required or mandatory, and that no other elements may be present. The term “consisting essentially of” indicates that any elements listed after the phrase are included, and that other elements than those listed may be included provided that those elements do not interfere with or contribute to the activity or action specified in the disclosure for the listed elements.

Unless otherwise specified, “a,” “an,” “the,” and “at least one” are used interchangeably and mean one or more than one.

As used herein, the term “each,” when used in reference to a collection of items, is intended to identify an individual term in the collection but does not necessarily refer to every term in the collection unless the context clearly dictates otherwise.

The recitations of numerical ranges by endpoints include all numbers subsumed within that range (e.g., 1 to 5 includes 1, 1.5, 2, 2.75, 3, 3.80, 4, 5, etc.).

For any method disclosed herein that includes discrete steps, the steps may be conducted in any feasible order. And, as appropriate, any combination of two or more steps may be conducted simultaneously.

The above summary of the present invention is not intended to describe each disclosed embodiment or every implementation of the present invention. The description that follows more particularly exemplifies illustrative embodiments. In several places throughout the application, guidance is provided through lists of examples, which examples can be used in various combinations. In each instance, the recited list serves only as a representative group and should not be interpreted as an exclusive list.

Reference throughout this specification to “one embodiment,” “an embodiment,” “certain embodiments,” or “some embodiments,” etc., means that a particular feature, configuration, composition, or characteristic described in connection with the embodiment is included in at least one embodiment of the disclosure. Thus, the appearances of such phrases in various places throughout this specification are not necessarily referring to the same embodiment of the disclosure. Furthermore, the particular features, configurations, compositions, or characteristics may be combined in any suitable manner in one or more embodiments.

Unless otherwise indicated, all numbers expressing quantities of components, molecular weights, and so forth used in the specification and claims are to be understood as being modified in all instances by the term “about.” Accordingly, unless otherwise indicated to the contrary, the numerical parameters set forth in the specification and claims are approximations that may vary depending upon the desired properties sought to be obtained by the present invention. At the very least, and not as an attempt to limit the doctrine of equivalents to the scope of the claims, each numerical parameter should at least be construed in light of the number of reported significant digits and by applying ordinary rounding techniques.

All headings are for the convenience of the reader and should not be used to limit the meaning of the text that follows the heading, unless so specified.

Notwithstanding that the numerical ranges and parameters setting forth the broad scope of the invention are approximations, the numerical values set forth in the specific examples are reported as precisely as possible. All numerical values, however, inherently contain a range necessarily resulting from the standard deviation found in their respective testing measurements.

### EXAMPLES

The following examples are illustrative only and are not intended to limit the scope of the application. Modifications will be apparent and understood by skilled artisans and are included within the spirit and under the disclosure of this application.

### Example 1: Identification of Focal Peak Problem

In this example, data shows that in experiments designed to sequence coding RNA, many reads of off-target abundant small noncoding RNA contaminate the desired sequencing information from the experiment.

In this example total RNA is the target nucleic acid in the sample, and RNA depletion involves four main steps: 1) hybridization, 2) RNase H treatment, 3) DNase treatment, and 4) target RNA clean up.

Hybridization is accomplished by annealing a defined DNA probe set to denatured RNA in a sample. A RNA sample, 10-100 ng, is incubated in a tube with 1 μL of a 1 μM/oligo DNA oligo probe set (probes corresponding to SEQ ID NOs: 1-333, as listed in Table 1), 3 μL of 5× Hybridization buffer (500 mM Tris HCl pH 7.5 and 1000 mM KCl), 2.5 μL of 100% formamide and enough water for a total reaction volume of 15 μL. The hybridization reaction is incubated at 95° C. for 2 min to denature the nucleic acids, slow cooled to 37° C. by decreasing temperature 0.1° C./sec and held at 37° C. No incubation time needed once the reaction reaches 37° C. The total time it takes for denaturation to reach 37° C. is about 15 min.

Following hybridization, the following components are added to the reaction tube for RNase H removal of the off-target RNA species from the DNA:RNA duplex; 4 μL 5× RNase H buffer (100 mM Tris pH 7.5, 5 mM DTT, 4 0 mM MgCl₂) and 1 μL RNase H enzyme. The enzymatic reaction is incubated at 37° C. for 30 min. The reaction tube can be held on ice.

Following the removal of the RNA from the DNA:RNA hybrid, the DNA probes are degraded. To the 20 μL reaction tube, the following components are added: 3 μL 10× Turbo DNase buffer (200 mM Tris pH 7.5, 50 mM CaCl₂, 20 mM MgCl₂), 1.5 μL Turbo DNase (Thermo Fisher Scientific) and 5.5 μL H₂O for a total volume of 30 μL. The enzymatic reaction is incubated at 37° C. for 30 min followed by 75° C. for 15 min. The 75° C. incubation can serve to fragment the target total RNA to desired insert sizes for use in downstream processing, in this example the target insert size is around 200 nt of total RNA. The timing of this incubation step can be adjusted depending on the insert size needed for subsequent reactions, as known to a skilled artisan. Following incubation, the reaction tube can be held on ice.

After hybridization of the probes to the off-target RNA, removal of the RNA, and removal of the DNA, the target total RNA in the sample can be isolated from the reaction conditions. The reaction tube is taken from 4° C. and allowed to come to room temperature and 60 μL of RNAClean XP beads (Beckman Coulter) are added and the reaction tube is incubated for 5 min. Following incubation, the tube is placed on a magnet for 5 min., after which the supernatant is gently removed and discarded. While still on the magnet, the beads with the attached total RNA are washed twice in 175 μL fresh 80% EtOH. After the second wash, the beads are spun down in a microcentrifuge to pellet the beads at the bottom of the tube, the tube is placed back on the magnet and the EtOH is removed, being careful to remove as much of the residual EtOH as possible without disturbing the beads. The beads are air dried for a few minutes, resuspended in 9.5 μL of ELB buffer (Illumina), allowed to sit a few more minutes at RT and placed back on the magnet to collect the beads. 8.5 μL of the supernatant is transferred to a fresh tube and placed on ice for additional downstream processing, such as created cDNA from the target total RNA.

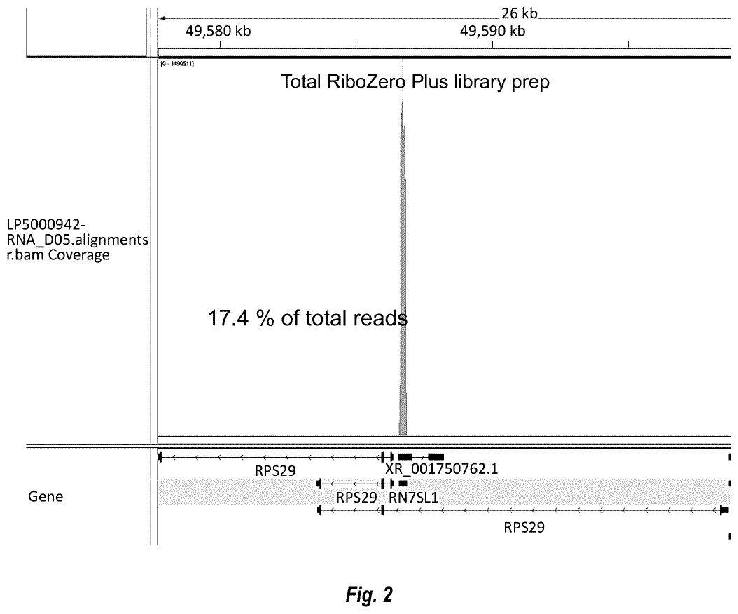

FIG. 2 shows that in an Integrative Genomics Viewer (IGV) plot for one sample. Integrative Genomics Viewer (IGV) is a desktop tool to visualize genomics data. Aligned RNA-seq reads were loaded into IGV and show library coverage per genomic position. There are almost 1.5 million reads stacked at one position and that this peak accounts for 17.4% of the total reads.

The signal recognition particle (SRP) is a cytoplasmic ribonucleoprotein complex that mediates cotranslational insertion of secretory proteins into the lumen of the endoplasmic reticulum. The SRP consists of 6 polypeptides (e.g., SRP19; MIM 182175) and a 7SL RNA molecule, such as RN7SL1, that is partially homologous to Alu DNA (Ullu and Weiner, Human genes and pseudogenes for the 7SL RNA component of signal recognition particle, PubMed 6084597, EMBO J. 3 (13): 33-3-10 (1984)). These are abundant small non-coding RNAs that dominate the sequencing reads.

Seven regions were identified from positions all across the genome and which were highly abundant, and these included primarily small non-coding RNA, as well as MALAT1 (a highly conserved large, infrequently spliced non-coding RNA which is highly expressed in the nucleus). Trying to remove these reads after sequencing resulted in a great deal of wasted sequencing. Therefore, depletion probes were designed to target six genes (RN7SK, RN7SL1, RN7SL5P, RPPH1, and SNORD3A, but not MALAT1). MALAT1 was not targeted because it is a long noncoding RNA that has been previously described as important in cancer. Table 1 provides information on the genes identified in the focal peak.

TABLE 1
Genes Identified in Focal Peak

| Gene_name | Gene_type | Gene_position |
|---|---|---|
| RN7SK | snRNA | chr6:52995621-52995948 |
| RN7SL5P | misc_RNA | chr9:9442060-9442380 |
| RPPH1 | ribozyme | chr14:20343075-20343407 |
| RN7SL1 | misc_RNA | chr14:49586580-49586878 |
| RN7SL2 | | |
| SNORD3A | snoRNA | chr17:19188016-19188714 |
| MALAT1 | lincRNA | chr11:65497762-65505019 |

SEQ ID NO: 7 shows the reverse complement for one of these sncRNAs, RN7SK, and alignment of depletion probes along its sequence, with 15 nucleotides between probe binding sites and 18 nucleotides at the end of the sequence. Other probes were designed using a similar method.

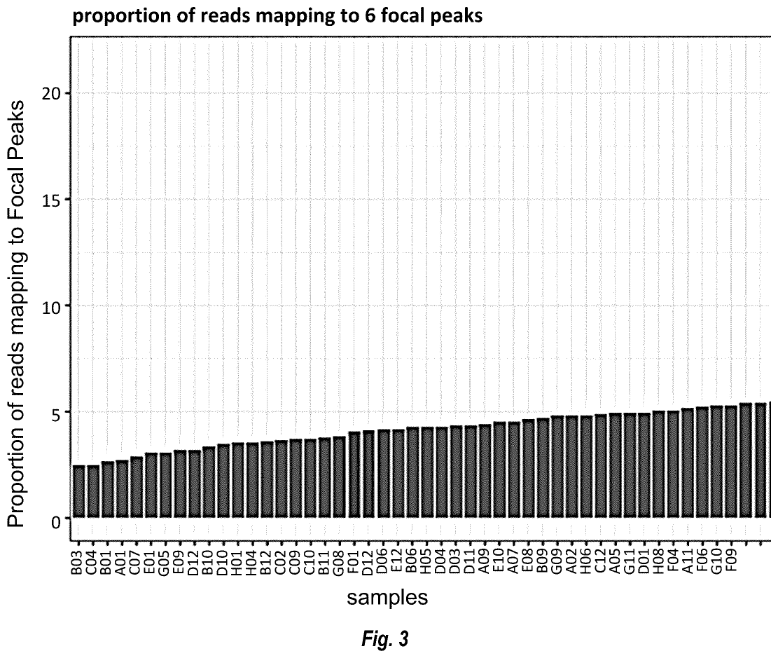

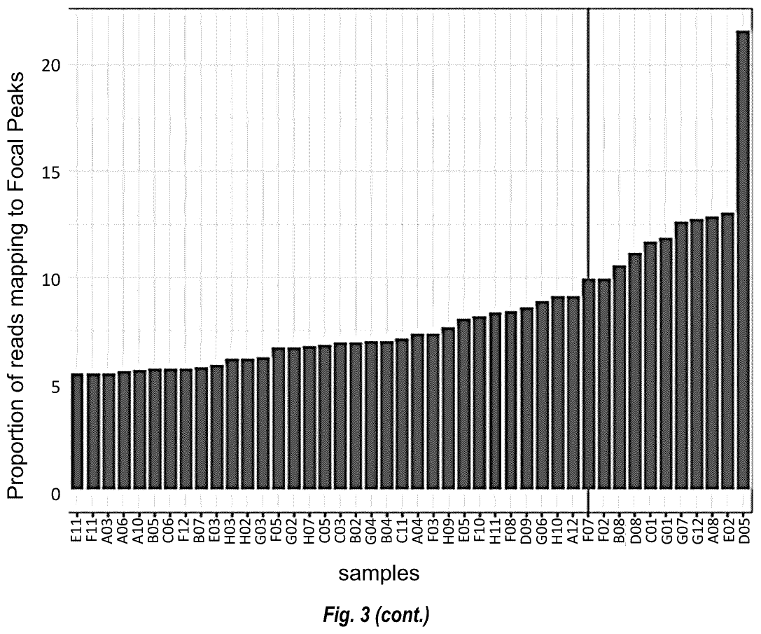

FIG. 3 also illustrates the problem of off-target RNA contaminating desired sequencing. 95 rare disease samples for which a diagnosis could not be made with whole genome sequencing were examined and the proportion of reads mapping to focal peaks was calculated for each sample. FIG. 3 shows the proportion of the reads that mapped to 7 focal peak genes across all 95 samples for this Rare and Undiagnosed Genetic Diseases (RUGD) project. From these samples, from 2% to 22% of all reads map into these 7 focal peak positions, with 9 samples having more than 10% of reads in focal peaks and 2 more samples having nearly 10% of reads in focal peaks.

The 9 worst affected samples with more than 10% of reads were used to regenerate new libraries using specifically designed probes to target these 6 genes on focal peaks to determine if we could alleviate the problem.

### Example 2: Depletion of Off-Target Abundant Small Noncoding RNA Species from a Sample

In this example, total RNA is the target nucleic acid in the sample, and RNA depletion involves four main steps: 1) hybridization, 2) depletion of off-target RNA, and 3) removal of probes.

PROBE HYBRIDIZATION: As a first step, probes were hybridized to the sample to bind to abundant small noncoding RNA. 100 ng of total RNA was diluted in 9 μl of nuclease-free ultrapure water into each well of a 96 well PCR plate. A Hybridize Probe Master Mix was prepared in a 1.7 ml tube on ice including 1.2 μl of DP1 and 3.6 μl of DB1. DP1 is a probe pool composed of 377 oligos all at 0.8 μM concentration per oligo in the pool. DB1 is a simple buffer at 5× concentration and composed of 500 mM Tris (pH 7.5) and 1000 mM KCl. For multiple samples, each volume was multiplied by the number of samples. These volumes produce more than 4 μl of Hybridize Probe Master Mix per well. Reagent overage is included in volumes to ensure accurate pipetting. The mixture was pipetted thoroughly to mix. Then, 4 μl of master mix was added to teach well.

Additionally, the probe set containing SEQ ID NOs: 8-39 (provided as a lyophilized pellet containing 50 pmol of each oligo) was dissolved by adding 50 μl of nuclease free water to the tube containing the probe set. The probe set and water was mixed, agitated, and spun down multiple times to dissolve fully. Upon resuspension, each oligo is present at about 1 μM per oligo.

Next, 2 μl of the dissolved probe mixture was added to each well, pipetted up and down 10 times to mix, and then sealed. The 96-well PCR plate was centrifuged at 280×g for 10 seconds to make sure any droplets that had sprayed onto the surfaces of the well during pipette mixing were spun down.

The plate was then placed on a preprogrammed thermal cycler and the HYB-DP1 program was run (the program comprises: heat to 95° C. for 2 min, then cool down to 37° C. by slowly ramping down the block temp 0.1° C. per second; hold at 37° C. until ready to add RDE and RDB). Each well had 15 μl sample.

RNA DEPLETION: As a second step, off-target RNA was depleted. An RNA Depletion Master Mix was prepared in a 1.7 ml tube on ice including 1.2 μl RDE (E. coli RNase H) and 4.8 μl RDB (containing 125 mM Tris pH 7.5, 5 mM DTT, and 40 mM MgCl₂). For multiple samples, each volume was multiplied by the number of samples. These volumes produce more than 5 μl RNA Depletion Master Mix per well. Reagent overage is included in volumes to ensure accurate pipetting. The mixture was pipetted thoroughly to mix. Then, the sealed sample plate was centrifuged at 280×g for 10 seconds. 5 μl of Depletion Master Mix was added to each well. The mixture was pipetted up and down 10 times to mix and then the 96-well plate was sealed. The 96-well PCR plate was centrifuged at 280×g for 10 seconds.

The plate was then placed on a preprogrammed thermal cycler and the RNA_DEP program was run (37° C. for 15 minutes). Each well had 20 μl sample.

PROBE REMOVAL: As a third step, the probes were removed. A Probe Removal Master Mix was prepared in a 1.7 ml tube one ice including 3.3 μl PRE (DNase I enzyme) and 7.7 μl PRB (4.3× buffer containing 257 mM Tris pH 7.5, 21.4 mM CaCl₂ and 25.7 mM MgCl₂). For multiple samples, each volume was multiplied by the number of samples. These volumes produce more than 10 μl RNA Depletion Master Mix per well. Reagent overage is included in volumes to ensure accurate pipetting. The mixture was pipetted thoroughly to mix. Then, the sealed sample plate was centrifuged at 280×g for 10 seconds. 10 μl of RNA Depletion Master Mix was added to each well. The mixture was pipetted up and down 10 times to mix and then the 96-well plate was sealed. The 96-well PCR plate was centrifuged at 280×g for 10 seconds. The reaction volume was 30 μl.

The plate was then placed on the preprogrammed thermal cycler and a program was run that pre-heated the lid to 100° C. Next the plate was incubated at 37° C. for 15 minutes, then 70° C. for 15 mins. The plate was then held at 4° C. Each well had 30 μl sample.

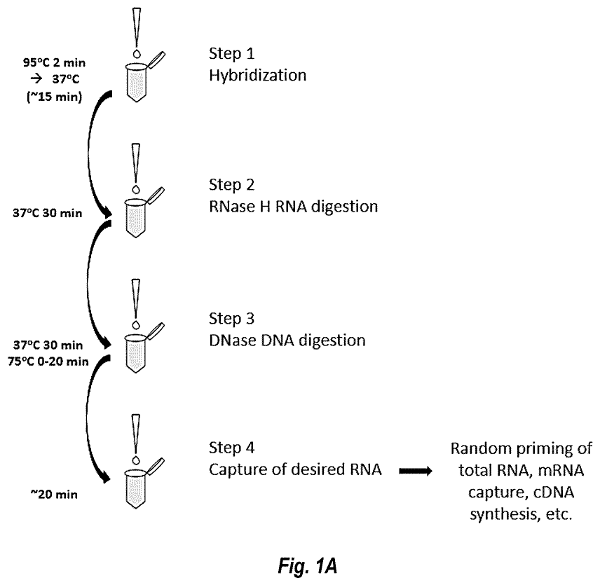

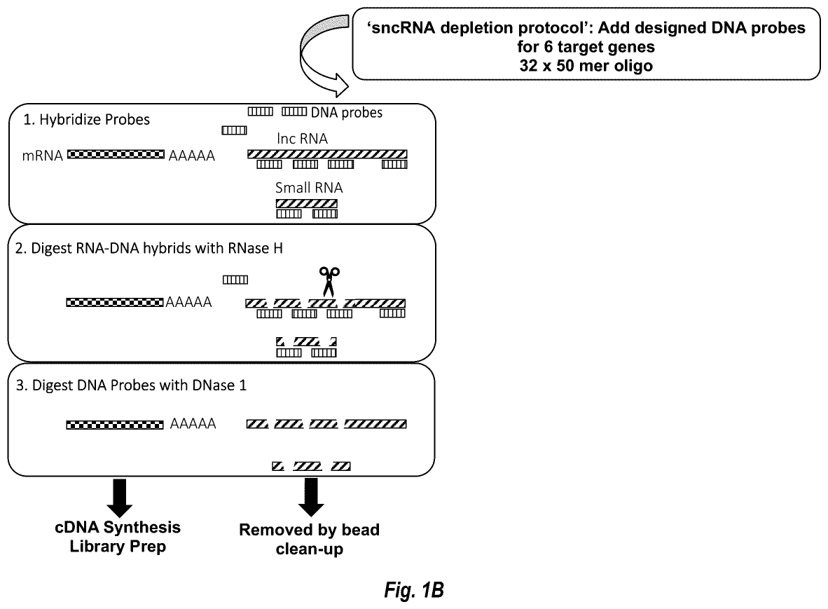

FIGS. 1A-B show the steps of these sncRNA depletion protocols schematically.

### Example 3: Evaluation of sncRNA Depletion WTS Libraries

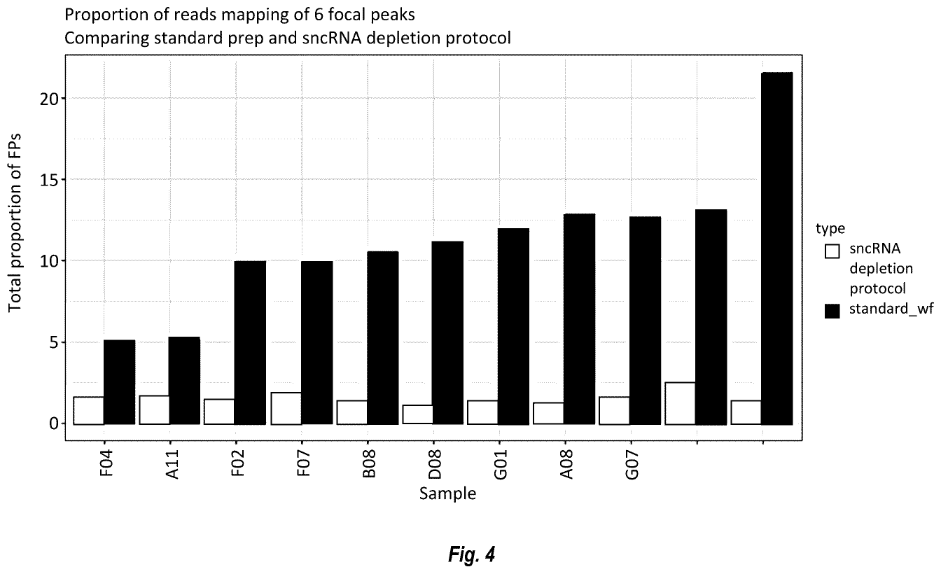

The new approach and set of depletion probes were tested on a set of blood RNAs. Blood RNAs originated from RUGD samples where whole genome sequencing could not provide a diagnosis. The aim of this experiment was to increase diagnostic yield using whole genome sequencing. 11 libraries were tested according to the standard workflow and also the sncRNA depletion protocol as set forth in Example 2. FIG. 4 shows results from testing of 11 different libraries with these two protocols. The black bars show the total proportion of focal peaks in sequencing reads using the standard workflow with from 5% to 22% of sequencing reads being from focal peaks. In comparison, the white bars show the total proportion of focal peaks in sequencing reads using the new sncRNA depletion protocol. The depletion probes used in the sncRNA depletion protocol were very effective in reducing the total proportion of focal peaks to about 1.5%. The 1.5% of reads mapping to focal peaks after the sncRNA depletion method represent the MALAT1 focal peak, which was not targeted. Eliminating many of the focal peak RNA species saves a great deal of sequencing resources.

### Example 4: Integrative Genomics Viewer (IGV) of RN7SL1 Standard Vs sncRNA Depletion Protocol

Example 4 was conducted according to the protocols in Example 1 (for the standard preparation) and Example 2 (for the sncRNA depletion preparation).

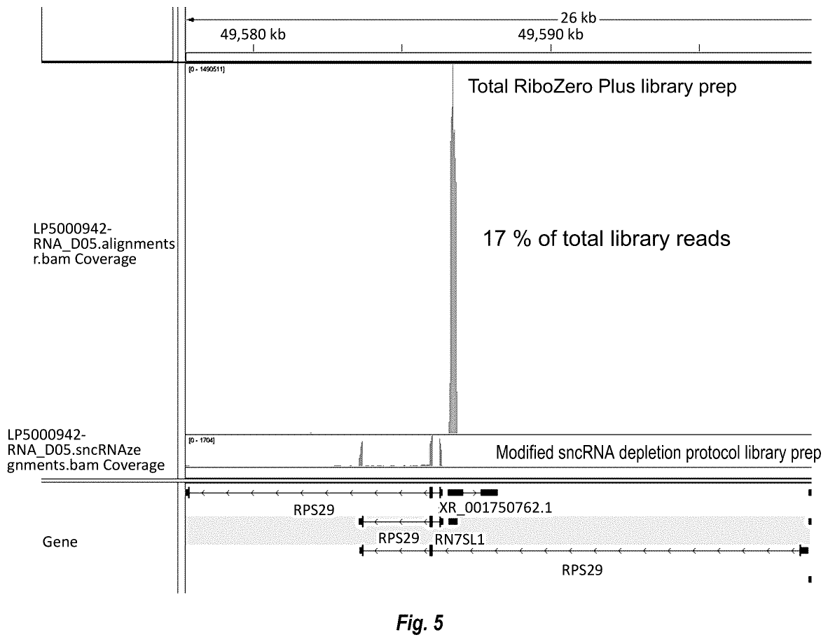

As a comparison to FIG. 2, FIG. 5 shows the differences between the plot shown in FIG. 2 and the results of the sncRNA depletion preparation, which clearly shows the absence of the RN7SL1 transcript which previously accounted for 17% of all sequencing reads. This shows that the presently employed depletion probes and method were able to deplete off-target RNA from the sample to improve sample quality before sequencing.

### Example 5: Evaluation of Key Library Metrics

Libraries were downsampled to 50 million reads to make all sequencing libraries comparable. Downsampling was performed using FASTQ Toolkit BaseSpace app by randomly sampling 50M paired reads from the original FASTQs. After obtaining downsampled FASTQs, RNA-seq alignment BaseSpace Sequence Hub (BSSH) app analysis was repeated.

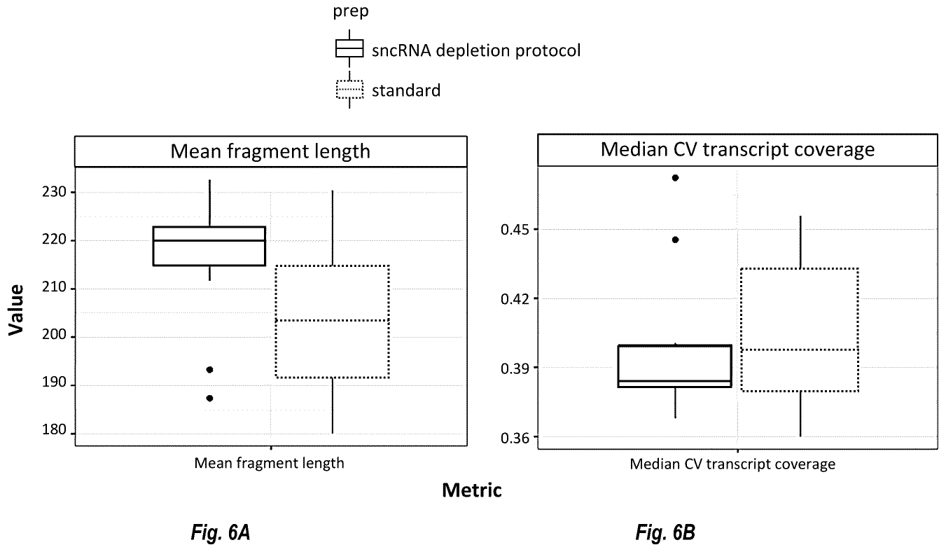

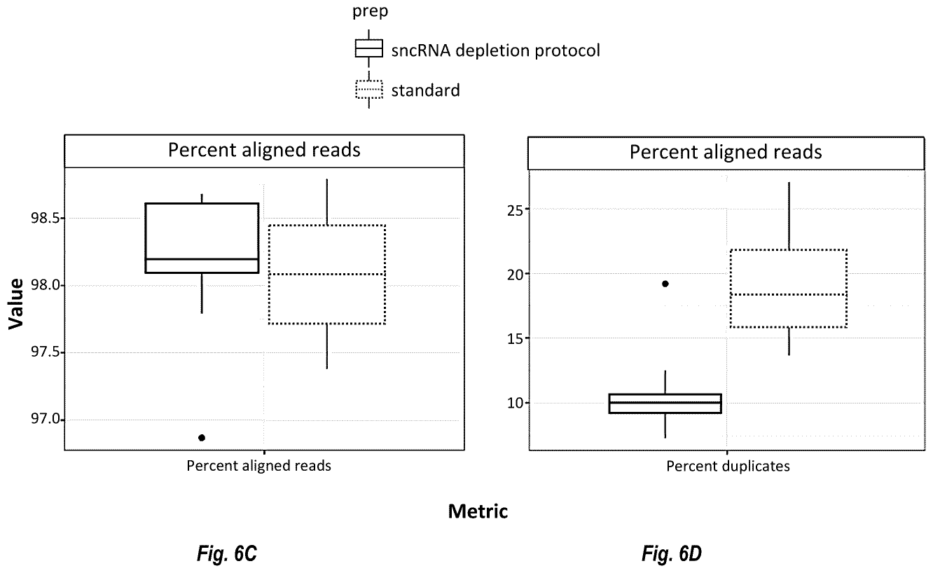

FIGS. 6A-D show key library metrics. FIG. 6A shows mean fragment length increased in the sncRNA depletion protocol in comparison to standard methods, providing further evidence of reduction in abundant small noncoding RNA. FIG. 6D shows that the percent of duplicate reads decreased in the sncRNA depletion protocol in comparison to standard methods. FIGS. 6B and 6C show that there was no significant change in median CV transcript coverage and percent aligned reads, measures of showing how well the sequencing covers the whole transcriptome.

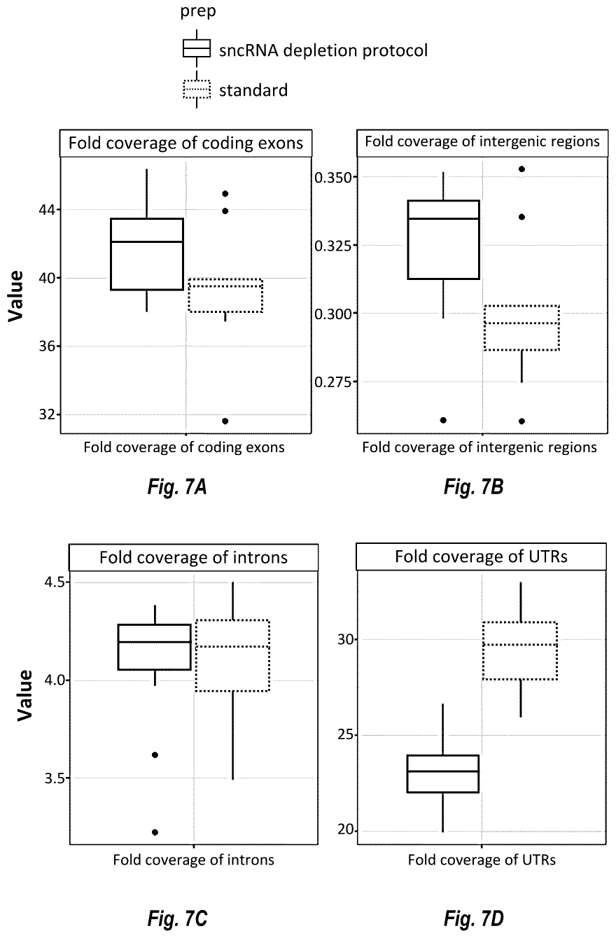

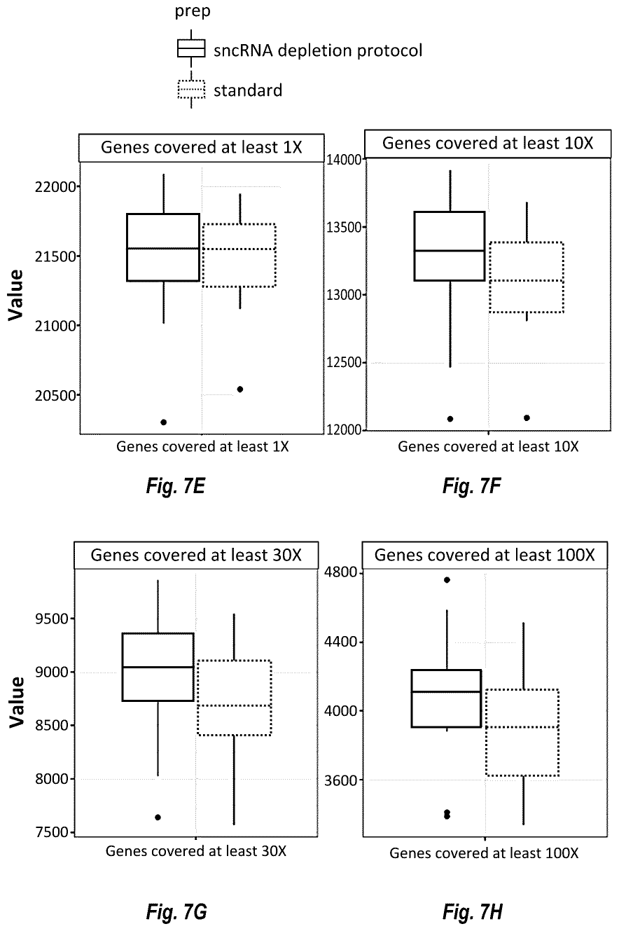

From the same experiment, FIGS. 7A-H show various gene coverage relating metrics, including fold coverage of coding exons (FIG. 7A), fold coverage of intergenic regions (FIG. 7B), fold coverage of introns (FIG. 7C), fold coverage of UTRs (FIG. 7D), and genes covered at least 1×, 10×, 30×, or 100× (FIGS. 7E-H). The strong reduction in percent reads mapping to UTRs (untranslated regions), as well as the increase in reads mapping to coding exons and intergenic regions, provides further support that this method was productive in depleting small noncoding RNA sequences. While genes covered at least 1× shows very little difference; however, an increase in stringency with the coverage shows this method results in gaining more useful sequence information.

Because off-target small RNAs were depleted, more reads aligned to other genes.

FIGS. 7E-7H show an increase in the number of genes with certain coverage in all panels between the standard preparation and the sncRNA depletion preparation. The difference is smaller in the 1× plot because there are already 21500 genes expressed at that level, reaching a limit of actively expressing genes.

At 30× or 100×, the number of genes is lower and because the off-target RNAs have been removed using the sncRNA depletion preparation, the difference between the two preparations is the most apparent.

### Example 6: The Addition of RNA Depletion Probes does not Distort Gene Expression

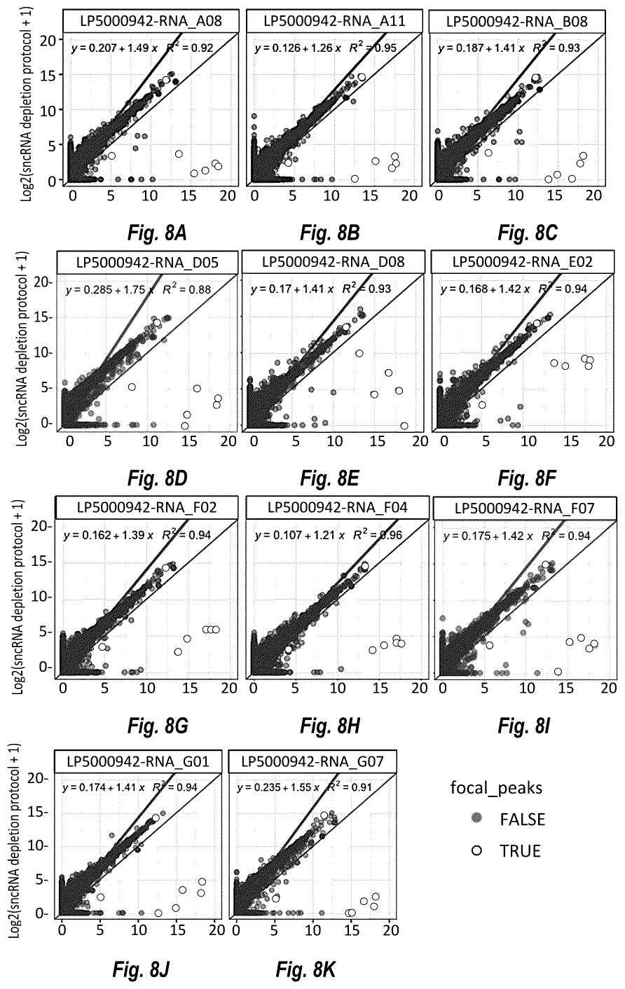

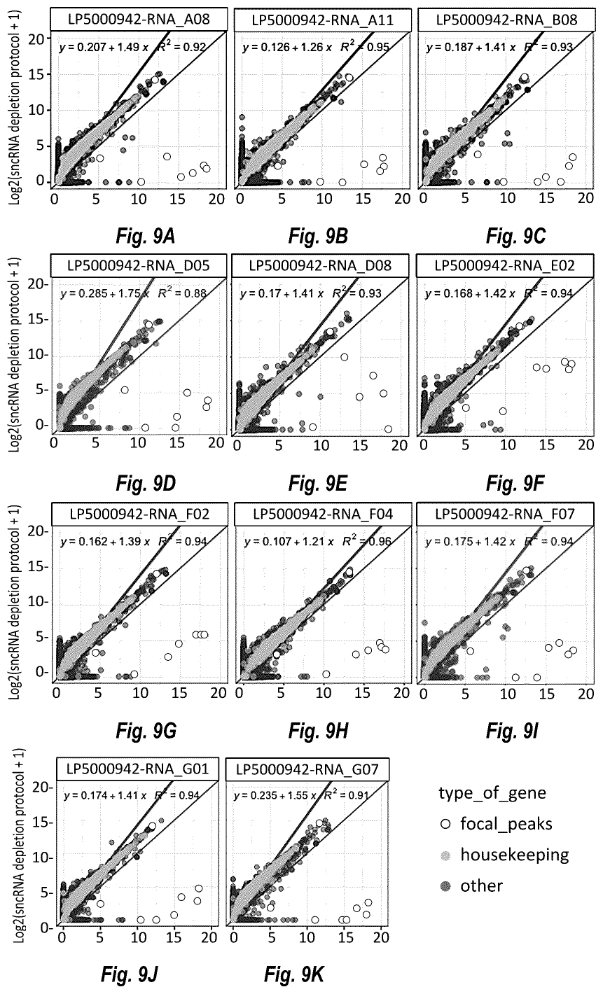

After downsampling all libraries to 50M paired reads, RNA-seq alignment app was run on BaseSpace (Illumina). As one of the processes, this app performs quantification of gene expression by using Salmon. The output of Salmon tells, for each gene, number of reads mapping to it and TPMs (transcripts per million). On FIGS. 8 and 9, Salmon quantification data obtained TPMs for libraries was plotted using standard RiboZero® protocol (X axis) and sncRNA depletion protocol (Y axes).

FIGS. 8A-K show distribution of transcripts per million (TPMs) corrected for read depth and length of gene for standard and sncRNA depletion preparations. Gene expression plots for 11 samples were plotted with the x axis showing the standard protocol and the y axis showing the depletion probe protocol. The white circles represent the focal peak genes that were targeted, which are generally significantly reduced. The solid gray circles represent genes that are not part of the focal peaks. The majority of genes are above the diagonal thin line with a slope of 1 where x=y, which means that they have a higher expression than the standard. The thicker line is the linear regression. This shows that expression of most genes was well replicated between the standard and sncRNA depletion preparations. FIG. 8 shows that highly expressed genes are further away from the diagonal and that highly expressed genes have more obvious increase in the expression. (In FIG. 8, some of the “false” focal peaks shown in gray appear black in the plot because of the density of overlapping genes plotted with gray focal peak dots.)

Housekeeping genes are a set of some 3000 genes from many different tissues from across the body, which should not change by more than 20% as they are involved in metabolism of the cell, energy production, and are genes that are active in all cells. FIGS. 9A-K show the same data as FIGS. 8A-K, reprocessed to highlight the housekeeping genes. The white circles represent the focal peak genes that were targeted, which are generally significantly reduced. The light gray circles represent housekeeping genes, while the dark gray circles (the same color as FIG. 8 “false”) represents other genes. This shows that housekeeping gene expression, like most genes, was well replicated between the standard and sncRNA depletion preparations.

### Example 7: The Effect of RNA Depletion

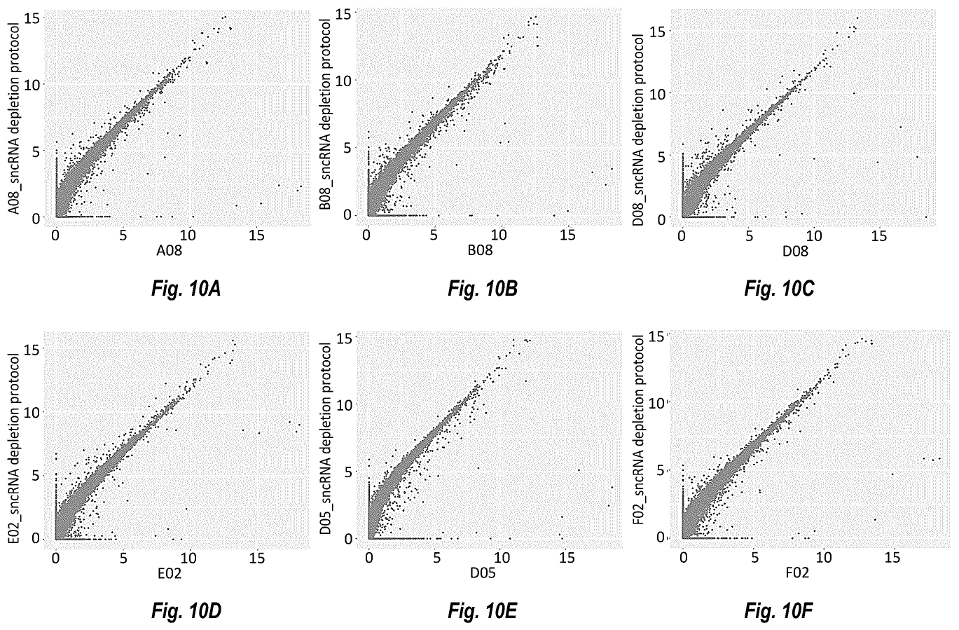

After downsampling all libraries to 50M paired reads, RNA-seq alignment app was run on BaseSpace. As one of the processes, this app performs quantification of gene expression by using Salmon. The output of Salmon tells, for each gene, number of reads mapping to it and TPMs (transcripts per million). On FIG. 10, Salmon obtained TPMs for libraries was plotted using standard RiboZero® protocol (X axis) and the sncRNA depletion protocol (Y axes)

FIGS. 10A-F show per gene log 2 (TPM+1) of depleted and non-depleted sequencing on the same samples. Gene expression plots for 6 representative views of 11 samples were plotted with the x axis showing the standard protocol and the y axis showing the depletion probe protocol.

Genes with TPM in 5-10 range in the nondepleted, standard protocol and 0 in the depleted protocol represent noncoding genes related to the genes targeted for depletion. Genes with TPMs in the 5-10 range in the depleted and 0 in nondepleted are noncoding genes, mainly small nucleolar RNAs. Specifically, these are transcripts not targeted for depletion, so they are detected at higher levels because the depletion targeted abundant small RNA and provides more reads and sensitivity for detecting the undepleted RNAs.

Analysis of this data showed that in a nondepleted method, a median of 23% of all sequencing reads were genes targeted for depletion, while after using the depletion method, only a median of 0.000006% of all sequencing reads were genes targeted for depletion. Likewise, analysis showed that using the nondepleted method, a median of 27% of all sequencing reads corresponded to the top ten expressed genes, while after using the depletion method only a median of 6% of all sequencing reads corresponded to the top ten expressed genes. This 6% is likely due to MALAT1, which was not targeted, and this significant reduction in the percent of sequencing reads corresponding to the top ten expressed genes shows significant improvement using this method.

### Example 8: The Effect of RNA Depletion

PanelApp creates gene lists for particular rare disease conditions. It narrows down the search for variants that caused the rare disease, with gene lists reviewed by external experts in these rare diseases. This panel comprises 3013 genes. Martin et al. Nature Genetics 51:1560-1565 (2019).

In this analysis, expression was quantified using Salmon. The output of Salmon tells, for each gene, number of reads mapping to it and TPMs (transcripts per million). TPM values were compared between control and depleted libraries to test if values changed using depletion method.

Results showed that 506 genes from the panel had a TPM of zero. A total of 18 genes had a lower TPM using the depleted method compared to the nondepleted method; however, 17 are very minor decreases in genes with very low expression and are likely noise rather than a meaningful decrease. Only Hemoglobin B (HBB) was decreased by ˜15. And 2489 genes had a higher TPM using the depleted method compared to the nondepleted method.

Table 2 shows the percentage of genes that have above zero expression across both methods, which is similar. But in the depleted set, nearly half of the PanelApp genes have transcripts per million above 10 (the level at where you can meaningfully detect mutations that affect gene splicing that might be causing the rare disease), but only about 19% using the nondepeleted method. This shows that the genes of interest have better representation in the sequencing data using the depletion method.

TABLE 2
PanelApp Genes: Depleted and Nondepleted
Median Transcripts Per Million (TPMs)

| | Depleted | Non-depleted |
|---|---|---|
| TPM >0 | 82.8% | 81.6% |
| TPM >10 | 46.8% | 19.1% |

In conclusion, this data shows that depletion of the small noncoding RNAs improves the data and there is better sequencing coverage of genes of interest. However, depleting small noncoding RNA can make it harder to compare data with data in other laboratories not using the depletion method.

Specifically, to allow more efficient transcript detection, investigators should remove highly abundant sncRNAs. Gene expression estimates were well correlated between the depletion and nondepletion methods. Depletion methods provided more power to detect aberrant splicing events. Depletion methods also improves sequencing data metrics including: (i) increasing TPMs, providing more reads on genes of interest, (ii) higher coding coverage, higher genes covered at 1×, 10×, 30×, or 100×, (iii) reducing the proportion of duplicates; and (iv) reducing the coverage at untranslated regions (UTRs).

### Example 9: The Effect of RNA Depletion from Commercially Available Human Bone Marrow RNA Samples

In this example, a commercially available pool of human bone marrow RNA samples (Thermo Fisher) was used. Libraries were prepared from these samples using the sncRNA depletion protocol depletion probes as described above.

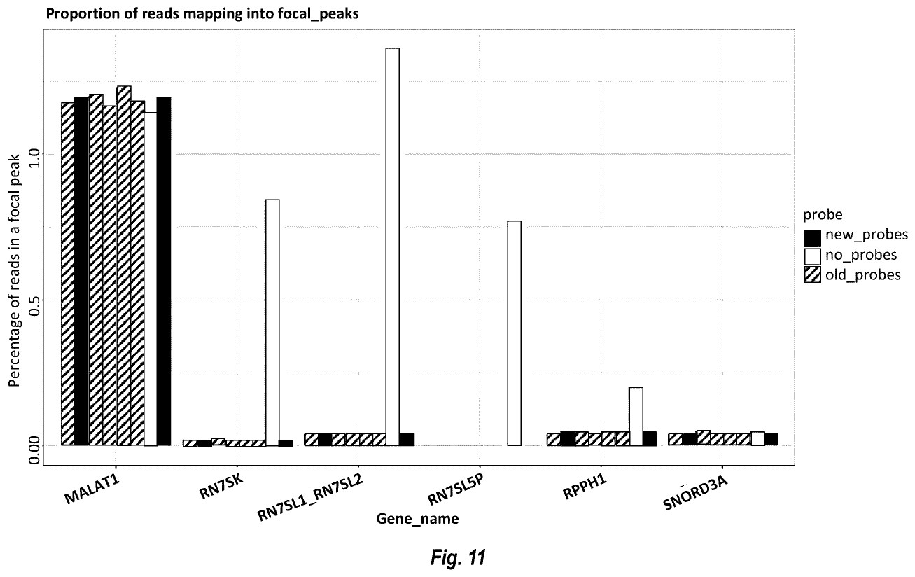

FIG. 11 shows the proportion of reads mapping into focal peaks of various genes. The white bars represent the library prep without the use of sncRNA depletion probes. The black bars and hashed bars are the same samples prepared with sncRNA depletion probes. The new probes (black) and old probes (hashed) refer to two different batches of the same probes, which both worked equally as well.

In contrast to Example 8 above, the particular bone marrow control sample that was used was not as affected by reads mapping in the focal peak genes, i.e., >1.5% reduced to 0.1% when probes were used. However, this data further illustrates that depletion of the small noncoding RNAs improves the data and there is better sequencing coverage of genes of interest.

1. A method for depleting off-target RNA molecules from a nucleic acid sample comprising:
providing a probe set comprising at least two DNA probes complementary to discontiguous sequences along the full length of the at least one off-target RNA molecule, wherein the at least one off-target RNA molecule comprises at least one small noncoding RNA chosen from RN7SK, RN7SL1, RN7SL2, RN7SL5P, RPPH1, SNORD3A;
(a) contacting a nucleic acid sample comprising at least one target RNA or DNA sequence and at least one off-target RNA molecule with the probe set, thereby hybridizing the DNA probes to the at least one off-target RNA molecule to form DNA:RNA hybrids, wherein each DNA:RNA hybrid is at least 5 bases apart, or at least 10 bases apart, along a given off-target RNA molecule sequence from any other DNA:RNA hybrid; and
(b) contacting the DNA:RNA hybrids with a ribonuclease that degrades the RNA from the DNA:RNA hybrids, thereby degrading the off-target RNA molecules in the nucleic acid sample to form a degraded mixture.

2. The method of claim 1, wherein at least one small noncoding RNA sequence is chosen from SEQ ID NOS: 1-6.

3. The method of claim 1, wherein at least one off-target RNA is chosen from a portion of SNORD3A that does not correspond to ALU.

4. The method of claim 1, wherein the off-target RNA is not MALAT1.

5. The method of claim 1, wherein a probe length is from 20 to 100 nucleotides.

6. The method of claim 1, wherein at least two probes in the probe set comprise any one of SEQ ID NOs: 8-39.

7. The method of claim 6, wherein the probe set comprises five or more, or 10 or more, or 25 or more sequences, or all of the sequences selected from SEQ ID NOs: 8-39.

8. The method of claim 1, wherein the probe set comprises five or more, or 10 or more, or 25 or more sequences, or all of the sequences selected from SEQ ID NOs: 40-467.

9. The method of claim 1, wherein the probe set comprises:
(a) two or more, or five or more, or 10 or more, or 25 or more, or 50 or more, or 100 or more, or 150 or more, or 200 or more, or 250 or more, or 300 or more, or 333 sequences selected from SEQ ID NOs: 40-372; or
(b) two or more, or five or more, or 10 or more, or 25 or more, or 50 or more, or 100 or more, or 150 or more, or 200 or more, or 250 or more, or 300 or more, or 350 or more, or 400 or more, or 428 sequences selected from SEQ ID NOs: 40-467; or
(c) two or more, or five or more, or 10 or more, or 25 or more, or 50 or more, or 100 or more, or 150 or more, or 200 or more, or 250 or more, or 300 or more, or 350 or more, or 377 sequences selected from SEQ ID NOs: 40-416; or
(d) (d) two or more, or five or more, or 10 or more, or 25 or more, or 50 or more, or 100 or more, or 150 or more, or 200 or more, or 250 or more, or 300 or more, or 350 or more, or 384 sequences selected from SEQ ID NOs: 40-372 and SEQ ID NOs: 416-467; or
(e) two or more, or five or more, or 10 or more, or 25 or more, or 44 sequences selected from SEQ ID NOs: 41-416; or
(f) two or more, or five or more, or 10 or more, or 25 or more, or 50 or more, or 51 sequences selected from SEQ ID NOs: 416-467;
(g) or a combination thereof.

10. The method of claim 1, wherein the at least two DNA probes bind to noncoding RNA molecules leaving a 15 base pair gap between probes.

11. The method of claim 1, further comprising: c) degrading any remaining DNA probes by contacting the degraded mixture with a DNA digesting enzyme, optionally wherein the DNA digesting enzyme is DNase I, to form a DNA degraded mixture; and d) separating the degraded RNA from the degraded mixture or the DNA degraded mixture.

12. The method of claim 1, wherein the contacting with the probe set comprises treating the nucleic acid sample with a destabilizer comprising formamide, wherein the formamide is present during the contacting with the probe set at a concentration of from about 10 to 45% by volume.

13. The method of claim 1, wherein the ribonuclease is RNase H or Hybridase.

14. The method of claim 1, wherein the probe set further comprises at least two DNA probes that hybridize to at least one off-target RNA molecule selected from 28S, 23S, 18S, 5.8S, 5S, 16S, 12S, HBA-A1, HBA-A2, HBB, HBB-B1, HBB-B2, HBG1, and HBG2.

15. The method of claim 1, wherein the probe set further comprises at least two DNA probes that hybridize to one or more off-target RNA molecules selected from HBA-A1, HBA-A2, HBB, HBG1, and HBG2 from hemoglobin, and 23S, 16S, and 5S from Gram positive or Gram negative bacteria.

16. The method of claim 1, wherein the probe set further comprises at least two DNA probes that hybridize to one or more off-target RNA molecules from an Archaea species.

17. The method of claim 1, wherein probes in the probe set to a particular off-target RNA molecule are complementary to about 65 to 85% of the sequence of the off-target RNA molecule, with gaps of at least 5, or at least 10, or 15 bases between each probe hybridization site.

18. A composition comprising a probe set comprising at least two DNA probes complementary to discontiguous sequences at least 5, or at least 10, or 15 bases apart along the full length of at least one off-target RNA molecule in a nucleic acid sample and a ribonuclease capable of degrading RNA in a DNA:RNA hybrid, wherein the off-target RNA comprises at least one small noncoding RNA chosen from RN7SK, RN7SL1, RN7SL2, RN7SL5P, RPPH1, SNORD3A.

19. A kit comprising a probe set comprising at least two DNA probes complementary to discontiguous sequences at least 5, or at least 10, or 15 bases apart along the full length of at least one off-target RNA molecule in a nucleic acid sample and a ribonuclease capable of degrading RNA in a DNA:RNA hybrid, wherein the off-target RNA comprises at least one small noncoding RNA chosen from RN7SK, RN7SL1, RN7SL2, RN7SL5P, RPPH1, SNORD3A.

20. A method of supplementing a probe set for use in depleting off-target RNA nucleic acid molecules from a nucleic acid sample comprising:
(a) contacting a nucleic acid sample comprising at least one RNA or DNA target sequence and at least one off-target RNA molecule from a first species with a probe set comprising at least two DNA probes complementary to discontiguous sequences along the full length of the at least one off-target RNA molecule from a second species, thereby hybridizing the DNA probes to the off-target RNA molecules to form DNA:RNA hybrids, wherein each DNA:RNA hybrid is at least 5 bases apart, or at least 10 bases apart, along a given off-target RNA molecule sequence from any other DNA:RNA hybrid, wherein the off-target DNA comprises at least one small noncoding RNA chosen from RN7SK, RN7SL1, RN7SL2, RN7SL5P, RPPH1, SNORD3A;
(b) contacting the DNA:RNA hybrids with a ribonuclease that degrades the RNA from the DNA:RNA hybrids, thereby degrading the off-target RNA molecules in the nucleic acid sample to form a degraded mixture;
(c) separating the degraded RNA from the degraded mixture;
(d) sequencing the remaining RNA from the sample;
(e) evaluating the remaining RNA sequences for the presence of off-target RNA molecules from the first species, thereby determining gap sequence regions; and
(f) supplementing the probe set with additional DNA probes complementary to discontiguous sequences in one or more of the gap sequence regions.
